(19) United States
(12) Patent Application Publication
(10) Pub. No.: US 2025/0101391 A1
(43) Pub. Date: Mar. 27, 2025

(54) HYBRID REVERSE TRANSCRIPTASES

(71) Applicant: Bio-Rad Laboratories, Inc., Hercules, CA (US)

(72) Inventors: Arkadiusz Bibillo, Walnut Creek, CA (US); Pranav Patel, Pleasanton, CA (US)

(21) Appl. No.: 18/974,072

(22) Filed: Dec. 9, 2024

Related U.S. Application Data
(63) Continuation of application No. 16/928,915, filed on Jul. 14, 2020, now Pat. No. 12,163,164.
(60) Provisional application No. 62/874,366, filed on Jul. 15, 2019.
(60) Provisional application No. 62/874,388, filed on Jul. 15, 2019.
(60) Provisional application No. 62/874,308, filed on Jul. 15, 2019.

(51) Int. Cl. C12N 9/12; C12N 1/20; C12R 1/01; C40B 40/06

(52) U.S. Cl. CPC C12N 9/1276; C12N 1/205; C07K 2319/80; C07K 2319/85; C12R 2001/01; C12Y 207/07049; C40B 40/06

(57) ABSTRACT

Hybrid reverse transcriptases are provided that comprise a non-retroviral retrotransposon, or a fragment of the non-retroviral retrotransposon having reverse transcriptase activity, joined to a nucleic acid binding protein. Also provided are methods of using the hybrid reverse transcriptases to prepare a cDNA molecule library.

This application is a continuation of U.S. patent application Ser. No. 16/928,915, filed Jul. 14, 2020, which claims the benefit of U.S. Provisional Application 62/874,308, filed on Jul. 15, 2019; U.S. Provisional Application 62/874,366, filed on Jul. 15, 2019; and U.S. Provisional Application 62/874,388, filed on Jul. 15, 2019, all of which are hereby incorporated by reference in their entirety.

The Sequence Listing for this application has been submitted electronically as an ASCII plain text file (XML) and is hereby incorporated by reference in its entirety. The XML file was created on Dec. 9, 2024, is named “BRP01073-US.xml” and is 225,819 bytes in size.

### BACKGROUND

A common technique used to study gene expression in living cells is to produce complementary deoxyribonucleic acid (cDNA) from a ribonucleic acid (RNA) molecule. This technique provides a means to study RNA from living cells that avoids the direct analysis of inherently unstable RNA. As a first step in cDNA synthesis, the RNA molecules from an organism are isolated from an extract of cells or tissues of the organism. After the messenger RNA (mRNA) is isolated, a RNA or DNA oligonucleotide sequence (i.e., a primer) is annealed to the isolated mRNA molecules and enzymes with reverse transcriptase activity are used to produce cDNA copies of the template RNA sequence. Reverse transcription of mRNA is used in many forms of gene expression analyses. For example, mRNA is reverse transcribed into cDNA for subsequent analysis by primer extension or polymerase chain reaction.

Many reverse transcriptases of retroviral origin are used commercially. Retroviral-derived reverse transcriptases have a RNA-directed DNA polymerase activity, a DNA-directed DNA polymerase activity, and an RNAse H activity. The RNAse H activity degrades RNA in an RNA:DNA duplex. Although retroviral-derived reverse transcriptases have been developed in which the RNAse H activity is absent, the enzymes have other properties that limit their utility. For example, retroviral-derived reverse transcriptases require a primer that can anneal to the RNA template, have low processivity (i.e., the enzyme dissociates from the RNA template before reaching the 5′ end), and the enzyme cannot transcribe through a region of the RNA template having secondary structure. Thus, retroviral-derived reverse transcriptases cannot transcribe long sequences of mRNA and have limited utility in such applications as single cell transcriptomics.

### Protein Ligation

Several technologies enable covalent conjugation of polypeptides at specific pre-determined sites. One example is the sortase system (Schmohl et al., 2014), whereby a short peptide (the sorting motif) is genetically fused to the C-terminus of one polypeptide and two glycine residues are genetically fused to the N-terminus of a second peptide (or vice versa). In the presence of the sortase enzyme, the two modified polypeptides are fused together. Other enzymatic protein ligase systems are butelase (Nguyen et al., 2014) or peptiligase (Toplak et al., 2016).

Another example is the in-frame addition of nucleotides encoding one or more cysteines to the C- or N-termini of polypeptides. When such free cysteine containing polypeptides are mixed under oxidizing conditions, they will form disulfide bridges. Such systems, however, suffer from the synthesis of many side-products and from instability of the disulfide bridge under reducing conditions.

Another example is the SpyTag/SpyCatcher (Reddington et al., 2015) system. Here, the concept of spontaneous isopeptide formation in naturally occurring proteins has been used to covalently attach one polypeptide to another. A domain from the Streptococcus pyogenes protein FbaB, which contains such isopeptide bond is split into two parts. One part, the SpyTag (SEQ ID NO: 6), is a 13 amino acid peptide that contains part (e.g., an aspartic acid) of the autocatalytic center. The other part, the SpyCatcher (SEQ ID NO: 9), is a 116 amino acid protein domain containing the other part (e.g., a lysine) of the center, promoted by a nearby glutamate or aspartate. Mixing those two polypeptides restores the autocatalytic center and leads to formation of the isopeptide bond, thereby covalently linking the SpyTag to the SpyCatcher (Zakeri et al., 2012). Further engineering has led to a shorter version of SpyCatcher with only 84 amino acids (SpyCatcher Short; SEQ ID NO: 11) as well as an optimized version, SpyTag002 (SEQ ID NO: 7) and SpyCatcher002 (SEQ ID NO: 10) with accelerated reaction (Li et al., 2014 and Keeble et al., 2017); both of which are hereby incorporated by reference in their entirety. A further modification of the system was the invention of SpyLigase (Fierer et al., 2014), which was achieved by splitting the FbaB domain into three parts: the SpyTag, the K-tag (SEQ ID NO: 13) and the SpyLigase. SpyLigase is a fragment of the FbaB domain comprising a glutamic acid residue that induces or catalyzes the formation of the isopeptide bond between the aspartate and lysine residues in SpyTag and K-tag, respectively.

Applications of such systems includes stabilization of proteins by circularization, vaccine generation, multimerization of proteins by integrating streptavidin/biotin with SpyTag/SpyCatcher (Reddington et al., 2015), affibody and Fab multimerization (Fierer et al., 2014), generation of antibodies from modules (Alam et al., 2017) as well as creation of antibody-drug conjugates (Siegmund et al., 2016), and generation of bispecific antibodies (Yumura et al., 2017). A similar system using the adhesin RrgA from Streptococcus pneumoniae was developed and termed SnoopTag/SnoopCatcher (Veggiani et al., 2016), with a later development of a SnoopLigase system (Buldun et al., 2018). The SnoopTag/SnoopCatcher technologies are hereby incorporated by reference in their entirety. Another system using Streptococcus pyogenes pilin subunit Spy0128 has also been developed and is called Isopeptag/Split Spy0128 (Abe et al., 2013). Yet another system derived from the Streptococcus dysgalactiae fibronectin-binding protein has also been developed and is called SdyTag/SdyCatcherDANG short (Tan et al., 2016). The Isopeptag/Split Spy0128 and SdyTag/SdyCatcherDANG short technologies are hereby incorporated by reference in their entirety.

### Biotinylation

Biotinylation is a method used to covalently attach biotin to biomolecules such as proteins and nucleic acids. Biotin has a small size that is unlikely to interfere with the function of the biomolecule. Biotin also binds tightly to streptavidin, resulting in a biotin-streptavidin complex that is resistant to extremes of heat, pH and proteolysis. The strong affinity between biotin and streptavidin is widely used in biotechnology, for example, to purify biomolecules via attachment to a solid support.

Proteins can be biotinylated chemically or enzymatically. Chemical biotinylation uses coupling chemistry to non-specifically attach biotin to the protein of interest, which can result in loss of protein activity. Unlike chemical biotinylation, enzymatic biotinylation allows biotin to be attached to one amino acid residue in the protein, which is less likely to have an impact on the protein activity. Beckett et al. (1999) developed a method to enzymatically biotinylate proteins by recombinantly fusing a 15 amino acid peptide (called AviTag or acceptor peptide) to a protein of interest. The acceptor peptide serves as a substrate for E. coli biotin holoenzyme synthetase or biotin ligase. When the protein fused to the acceptor peptide is incubated with biotin ligase in the presence of biotin and ATP, the biotin ligase catalyzes biotinylation of a lysine group in the acceptor peptide.

### SUMMARY

The invention relates to hybrid reverse transcriptases having improved properties that overcome current limitations in the field and, among other things, result in increased processivity. Hybrid reverse transcriptases of the invention comprise a protein having reverse transcriptase activity and a protein that binds to a nucleic acid template (i.e., a nucleic acid binding protein). The proteins are covalently joined to each other via recombinant methods or via protein ligation, or are non-covalently joined to a biotin-binding protein. A preferred protein having reverse transcriptase activity transcribes RNA with a primer that is not complementary to the 3′ end of the template RNA, can jump from the 5′ end of the template RNA to a second template (an “acceptor-adaptor”) to continue transcription, and can transcribe through a region of the RNA template having secondary structure. The nucleic acid binding protein enhances the ability of the hybrid reverse transcriptase to bind to the template RNA and can therefore improve the processivity of the enzyme. Thus, these properties of the hybrid reverse transcriptases allow the enzymes to bind to and transcribe mRNA sequences (e.g., long mRNA sequences) for subsequent amplification and sequence analysis.

In an embodiment, a hybrid reverse transcriptase comprises a non-retroviral retrotransposon, or a fragment of the non-retroviral retrotransposon having reverse transcriptase activity, recombinantly joined to a nucleic acid binding protein or a fragment of the nucleic acid binding protein. The nucleic acid binding protein can be a single stranded deoxyribonucleic acid (ssDNA) binding protein, a double stranded deoxyribonucleic acid (dsDNA) binding protein, a nucleic acid tag binding protein or fragments thereof.

The non-retroviral retrotransposon, or the fragment of the non-retroviral retrotransposon, is joined either directly or via a linker to the nucleic acid binding protein or a fragment of the nucleic acid binding protein. The nucleic acid binding protein can be, for example, Sso7d, a fragment of Sso7d, Cren7, a fragment of Cren7, Sulfolobus SSB, a fragment of Sulfolobus SSB, or an anti-nucleic acid tag antibody (e.g., an anti-Digoxigenin antibody).

In some embodiments, two or more nucleic acid binding proteins, or fragments of two or more nucleic acid binding proteins, are joined to the N-terminus or C-terminus of the non-retroviral retrotransposon or the fragment of the non-retroviral retrotransposon. The two or more nucleic acid binding proteins, or fragments of two or more nucleic acid binding proteins, can be in sequential or random order.

In some embodiments, the non-retroviral retrotransposon, or the fragment of the non-retroviral retrotransposon, is covalently joined to the nucleic acid binding protein, or the fragment of the nucleic acid binding protein.

In certain embodiments, the hybrid reverse transcriptase is a multimeric enzyme comprising two or more non-retroviral retrotransposons, or two or more fragments of the non-retroviral retrotransposon having reverse transcriptase activity joined with or without a linker. In some embodiments, the two or more non-retroviral retrotransposons, or the two or more fragments of the non-retroviral retrotransposon, are covalently joined.

In some embodiments, the non-retroviral retrotransposon, or the fragment of the non-retroviral retrotransposon, is an R2 reverse transcriptase or a fragment of the R2 reverse transcriptase (e.g., a recombinant R2 enzyme minus an N-terminal DNA binding domain), respectively. Some embodiments of the R2 reverse transcriptase comprise domains from two or more arthropods.

In certain embodiments, the linker lacks a secondary structure. Exemplary linkers include, but are not limited to, VGTVGTGGGSGGASTAL, VGTVGTGGGSEAAAKGGASTAL, VGTGGGSEAAAKGGASTAL, VGTGGGSGGGEAAAKEAAAKSGGGS, VGTGGGSGGGEAAAKEAAAKSGGGSA, VGTGGGSGGGTGGGS, VGTGGGSGGGTGGGSA, (GGGS)ₙ, (GGS)ₙ, (GGGGS)ₙ, and (EAAAK)ₙ where n is 1, 2, 3, 4, or 5.

In some embodiments, the hybrid reverse transcriptase comprises a purification tag at an N-terminus or a C-terminus.

In certain embodiments, the hybrid reverse transcriptase comprises a non-retroviral retrotransposon having at least 75% sequence identity to SEQ ID NO: 1 joined to a nucleic acid binding protein or a fragment of the nucleic acid binding protein. In some embodiments, the hybrid reverse transcriptase comprises a fragment of a non-retroviral retrotransposon having at least 75% sequence identity to SEQ ID NO: 2 joined to a nucleic acid binding protein or a fragment of the nucleic acid binding protein.

In some embodiments, the hybrid reverse transcriptase comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 98, SEQ ID NO: 99, or SEQ ID NO: 100 or a sequence with at least 75% identity to SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 98, SEQ ID NO: 99, or SEQ ID NO: 100.

In an embodiment, a hybrid reverse transcriptase comprises a non-retroviral retrotransposon, or a fragment of the non-retroviral retrotransposon having reverse transcriptase activity, in either case comprising a first binding motif and a nucleic acid binding protein, or a fragment of the nucleic acid binding protein, in either case comprising a second binding motif. The first binding motif is covalently joined to the second binding motif via protein ligation.

In some embodiments, the binding motif is located at a C terminus or an N-terminus of the non-retroviral retrotransposon, the fragment of the non-retroviral retrotransposon, the nucleic acid binding protein, or the fragment of the nucleic acid binding protein. In certain embodiments, the binding motif is joined to the non-retroviral retrotransposon, the fragment of the non-retroviral retrotransposon, the nucleic acid binding protein, or the fragment of the nucleic acid binding protein via a linker.

In some embodiments, the hybrid reverse transcriptase is a multimeric enzyme comprising a first non-retroviral retrotransposon, or a first fragment of the non-retroviral retrotransposon having reverse transcriptase activity, in either case comprising a first binding motif and a second non-retroviral retrotransposon, or a second fragment of the non-retroviral retrotransposon having reverse transcriptase activity, in either case comprising a second binding motif. The first binding motif is covalently joined to the second binding motif via protein ligation.

In some embodiments, the first binding motif comprises SEQ ID NO: 48, 49, or 50 or a sequence with at least 60% identity to 48, 49, or 50 and the second binding motif comprises SEQ ID NO: 51, 52, 53, 54, or 55 or a sequence with at least 60% identity to 51, 52, 53, 54, or 55; or the first binding motif comprises SEQ ID NO: 51, 52, 53, 54, or 55 or a sequence with at least 60% identity to 51, 52, 53, 54, or 55 and the second binding motif comprises SEQ ID NO: 48, 49, or 50 or a sequence with at least 60% identity to 48, 49, or 50. In certain embodiments, the first binding motif comprises SEQ ID NO: 56, 57, or 58 or a sequence with at least 60% identity to 56, 57, or 58 and the second binding motif comprises SEQ ID NO: 59, 60, or 61 or a sequence with at least 60% identity to 59, 60, or 61; or the first binding motif comprises SEQ ID NO: 59, 60, or 61 or a sequence with at least 60% identity to 59, 60, or 61 and the second binding motif comprises SEQ ID NO: 56, 57, or 58 or a sequence with at least 60% identity to 56, 57, or 58.

In certain embodiments, the first binding motif comprises a sortase A or B recognition domain comprising the amino acid sequence: LPTGAA (SEQ ID NO: 62), LPTGGG (SEQ ID NO: 63), LPKTGG (SEQ ID NO: 64), LPETG (SEQ ID NO: 65), LPXTG (SEQ ID NO: 66) or LPXTG(X)ₙ (SEQ ID NO: 67), where X is any amino acid, and n is 0, 1, 2, 3, 4, 5, 7, 8, 9, 10, in the range of 0-5 or 0-10, or any integer up to 100, or NPX1TX2 (SEQ ID NO: 68), where X1 is glutamine or lysine; X2 is asparagine or glycine; N is asparagine; P is proline and T is threonine, and the second binding motif comprises a sortase A or B bridging domain comprising: Gly, (Gly)₂, (Gly)₃, (Gly)₄, or (Gly)ₓ, where x is an integer of 1-20.

In some embodiments, the hybrid reverse transcriptase comprises a non-retroviral retrotransposon having at least 75% sequence identity to SEQ ID NO: 1 joined via protein ligation to a nucleic acid binding protein or a fragment of the nucleic acid binding protein. In some embodiments, the hybrid reverse transcriptase comprises a fragment of a non-retroviral retrotransposon having at least 75% sequence identity to SEQ ID NO: 2 joined via protein ligation to a nucleic acid binding protein or a fragment of the nucleic acid binding protein.

In an embodiment, a hybrid reverse transcriptase comprises a non-retroviral retrotransposon, or a fragment of the non-retroviral retrotransposon having reverse transcriptase activity, in either case comprising a biotinylated first acceptor peptide and a nucleic acid binding protein, or a fragment of the nucleic acid binding protein, comprising a biotinylated second acceptor peptide. The biotinylated first and second acceptor peptides are joined to a biotin-binding protein via non-covalent interactions. The nucleic acid binding protein can be a single stranded deoxyribonucleic acid (ssDNA) binding protein or a double stranded deoxyribonucleic acid (dsDNA) binding protein. For example, the nucleic acid binding protein can be Sso7d, a fragment of Sso7d, Cren7, a fragment of Cren7, Sulfolobus SSB, or a fragment of Sulfolobus SSB.

In certain embodiments, the acceptor peptide is located at a C terminus or an N-terminus of the non-retroviral retrotransposon, the fragment of the non-retroviral retrotransposon, the nucleic acid binding protein, or the fragment of the nucleic acid binding protein. In some embodiments, the acceptor peptide is joined to the non-retroviral retrotransposon, the fragment of the non-retroviral retrotransposon, the nucleic acid binding protein, or the fragment of the nucleic acid binding protein via a linker.

In some embodiments, the hybrid reverse transcriptase comprises a first non-retroviral retrotransposon, or a first fragment of the non-retroviral retrotransposon having reverse transcriptase activity, comprising a biotinylated first acceptor peptide and a second non-retroviral retrotransposon, or a second fragment of the non-retroviral retrotransposon having reverse transcriptase activity, comprising a biotinylated second acceptor peptide. The biotinylated first and second acceptor peptides are non-covalently joined to a biotin-binding protein via non-covalent interactions.

In some embodiments, the first and second acceptor peptides comprise SEQ ID NO: 85, 86, 87, 88, or 89.

In certain embodiments, the hybrid reverse transcriptase comprises a non-retroviral retrotransposon having at least 75% sequence identity to SEQ ID NO: 1 and a nucleic acid binding protein, or a fragment of the nucleic acid binding protein, non-covalently joined to a biotin-binding protein. In some embodiments, the hybrid reverse transcriptase comprises a fragment of a non-retroviral retrotransposon having at least 75% sequence identity to SEQ ID NO: 2 and a nucleic acid binding protein, or a fragment of the nucleic acid binding protein, non-covalently joined to a biotin-binding protein.

In some embodiments, the biotin-binding protein is streptavidin, traptavidin, or neutravidin.

In some embodiments, the hybrid reverse transcriptase comprises a purification tag at an N-terminus or a C-terminus of the non-retroviral retrotransposon, the fragment of the non-retroviral retrotransposon, the nucleic acid binding protein, and/or the fragment of the nucleic acid binding protein.

Nucleic acid constructs comprising a polynucleotide sequence encoding the periplasmic fusion proteins are also provided.

Also provided are a pair of nucleic acid constructs comprising a first nucleic acid construct comprising a polynucleotide sequence encoding a non-retroviral retrotransposon, or a fragment of the non-retroviral retrotransposon having reverse transcriptase activity, fused to a first binding motif, and a second nucleic acid construct comprising a polynucleotide sequence encoding a nucleic acid binding protein, or a fragment of the nucleic acid binding protein, fused to a second binding motif. Also provided are a pair of nucleic acid constructs comprising a first nucleic acid construct comprising a polynucleotide sequence encoding a first non-retroviral retrotransposon, or a first fragment of the non-retroviral retrotransposon having reverse transcriptase activity, fused to a first binding motif, and a second nucleic acid construct comprising a polynucleotide sequence encoding a second non-retroviral retrotransposon, or a second fragment of the non-retroviral retrotransposon having reverse transcriptase activity fused to a second binding motif. For both pairs of nucleic acid constructs, the first binding motif and the second binding motif form a covalent bond via protein ligation when brought into contact with one another either spontaneously or with the help of an enzyme.

Also provided are a pair of nucleic acid constructs comprising a first nucleic acid construct comprising a polynucleotide sequence encoding a non-retroviral retrotransposon, or a fragment of the non-retroviral retrotransposon having reverse transcriptase activity fused to a first acceptor peptide, and a second nucleic acid construct comprising a polynucleotide sequence encoding a nucleic acid binding protein, or a fragment of the nucleic acid binding protein, fused to a second acceptor peptide. Also provided are a pair of nucleic acid constructs comprising a first nucleic acid construct comprising a polynucleotide sequence encoding a first non-retroviral retrotransposon, or a first fragment of the non-retroviral retrotransposon having reverse transcriptase activity fused to a first acceptor peptide, and a second nucleic acid construct comprising a polynucleotide sequence encoding a second non-retroviral retrotransposon, or a second fragment of the non-retroviral retrotransposon having reverse transcriptase activity fused to a second acceptor peptide. For both pairs of nucleic acid constructs, the first acceptor peptide and the second acceptor peptide are biotinylated in the presence of biotin ligase and non-covalently bind to a biotin-binding protein when brought into contact with one another.

The invention contemplates vectors comprising nucleic acid constructs disclosed herein and host cells comprising the nucleic acid constructs and/or the vectors.

Methods in which the hybrid reverse transcriptase comprises a dsDNA binding protein are also provided for producing a complementary deoxyribonucleic acid (cDNA) molecule or a cDNA library. In an exemplary method, a template RNA molecule and free nucleotides, are contacted with: a primer that is complementary to the template RNA molecule; an acceptor-adapter; and a hybrid reverse transcriptase comprising a dsDNA binding protein or a fragment of a dsDNA binding protein. The next step of the method comprises allowing the hybrid reverse transcriptase to transcribe the template RNA molecule under conditions effective to produce a cDNA molecule complementary to the RNA molecule and, optionally, to the acceptor-adapter, wherein the hybrid reverse transcriptase jumps to a 3′-end of the acceptor-adapter upon reaching a 5′ end of the RNA molecule.

In some embodiments, a method of preparing a cDNA molecule library comprises fragmenting a template RNA molecule to produce RNA fragments. In the next step, a 2′, 3′-cyclic phosphate and a 3′-phosphate are removed from the RNA fragments, thereby generating dephosphorylated RNA fragments. A poly-A tail is then added to the dephosphorylated RNA fragments to form poly-A tailed RNA fragments. In the next step, a primer-adapter comprising an oligo-T sequence; an acceptor-adapter; free nucleotides; and a hybrid reverse transcriptase comprising a dsDNA binding protein, or a fragment of a dsDNA binding protein, are added to the poly-A tailed RNA fragments. The hybrid reverse transcriptase is then allowed to transcribe the poly-A tailed RNA fragments under conditions effective to produce a cDNA molecule library, wherein the hybrid reverse transcriptase jumps to a 3′-end of the acceptor-adapter upon reaching a 5′ end of the poly-A tailed RNA fragments. In this method, the dephosphorylation and poly (A) addition steps can be combined into one step.

In some embodiments, a method of preparing a cDNA molecule library comprises annealing a primer adapter comprising a poly (T) tail to a template RNA molecule comprising a poly (A) tail, thereby generating an annealed RNA molecule. Next, free nucleotides, the annealed RNA molecule, an acceptor-adapter, and a hybrid reverse transcriptase comprising a a dsDNA binding protein, or a fragment of a dsDNA binding protein, are mixed. The hybrid reverse transcriptase is then allowed to transcribe the annealed RNA molecule at a temperature from about 12° C. to about 42° C. to produce a cDNA molecule library, wherein the hybrid reverse transcriptase jumps to a 3′-end of the acceptor-adapter upon reaching a 5′ end of the annealed RNA molecule. The template RNA molecule can be a messenger RNA molecule.

In some embodiments, a method of preparing a cDNA molecule library comprises annealing one or more random primer adapters to template RNA molecules comprising a poly (A) tail, thereby generating annealed RNA molecules. Next, free nucleotides, the annealed RNA molecules, one or more acceptor-adapters, and a hybrid reverse transcriptase comprising a dsDNA binding protein, or a fragment of a dsDNA binding protein, are mixed. The hybrid reverse transcriptase is then allowed to transcribe the annealed RNA molecule without thermal cycling to produce a cDNA molecule library, wherein the hybrid reverse transcriptase jumps to a 3′-end of the acceptor-adapter upon reaching a 5′ end of the annealed RNA molecules. In this method, all the steps can be performed in a single reaction vessel. The template RNA molecules can comprise messenger RNAs, ribosomal RNAs, transfer RNAs (tRNAs), micro RNAs, and/or long non-coding RNAs.

Methods in which the hybrid reverse transcriptase comprises a ssDNA binding protein are also provided for producing a complementary deoxyribonucleic acid (cDNA) molecule or a cDNA library. In some embodiments, a method of preparing a cDNA molecule comprises contacting a template RNA molecule and free nucleotides with: a primer that is not complementary to the template RNA molecule, an acceptor-adapter, and a hybrid reverse transcriptase comprising a ssDNA binding protein or a fragment of a ssDNA binding protein. The hybrid reverse transcriptase is then allowed to transcribe the template RNA molecule under conditions effective for producing a cDNA molecule complementary to the RNA molecule and, optionally, to the acceptor-adapter, wherein the hybrid reverse transcriptase jumps to a 3′-end of the acceptor-adapter upon reaching a 5′ end of the RNA molecule. The primer can be ssDNA or ssRNA. In some embodiments in which the hybrid reverse transcriptase is a fusion protein, the ssDNA binding protein is an anti-Digoxigenin antibody and the primer comprises a Digoxigenin tag.

In some embodiments, a method of preparing a cDNA molecule library comprises fragmenting a template RNA molecule to produce RNA fragments. The RNA fragments and free nucleotides are then contacted with: a primer-adapter that is not complementary to the RNA fragments, an acceptor-adapter, and a hybrid reverse transcriptase comprising a ssDNA binding protein or a fragment of a ssDNA binding protein. In the next step of the method, the hybrid reverse transcriptase is allowed to transcribe the RNA fragments under conditions effective to produce a cDNA molecule library, wherein the hybrid reverse transcriptase jumps to a 3′-end of the acceptor-adapter upon reaching a 5′ end of the RNA fragments. All the steps of the method can be combined into one step. Any or all of the steps can be performed in a partition.

In some embodiments, a method of preparing a cDNA molecule library comprises providing a partition comprising: a cell comprising template RNA molecules, nucleotides, a primer adapter that is not complementary to the RNA molecules, an acceptor-adapter, an endonuclease, and a hybrid reverse transcriptase comprising a ssDNA binding protein or a fragment of a ssDNA binding protein. In the partition, template RNA molecules are then released from the cell, the template RNA molecules are fragmented to form RNA fragments, and the hybrid reverse transcriptase is allowed to transcribe the RNA fragments under conditions effective to produce a cDNA molecule library, wherein the hybrid reverse transcriptase jumps to a 3′-end of the acceptor-adapter upon reaching a 5′ end of the dephosphorylated RNA fragments.

The method may further comprise removing one or more non-annealed primer adapter after the last step. The one or more non-annealed primer-adapter can be removed with an immobilized poly A oligo.

In some embodiments, the jumping of the hybrid reverse transcriptase to the 3′-end of the acceptor-adapter is independent of sequence identity between the template RNA molecule(s) and the acceptor-adapter. The cDNA molecule or cDNA library can be prepared in about 2 hours or less.

In certain embodiments, the method further comprises amplifying the cDNA molecule(s) by polymerase chain reaction to form one or more amplicons. The amplifying can be performed at a temperature sufficient to inactivate the reverse transcriptase. In some embodiments, producing and amplifying the cDNA molecule(s) is performed in the same reaction vessel.

In some embodiments, the method further comprises adding a label to the template RNA molecule(s) to generate a labeled cDNA molecule(s). In certain embodiments, the method further comprises sequencing the labeled cDNA molecule(s).

In certain embodiments, the acceptor-adapter, the primer adapter, or both the acceptor-adapter and primer adapter can comprise a nucleotide analogue that stops the reverse transcription by the hybrid reverse transcriptase. In some embodiments, the nucleotide analogue can be at the 5′ end of the acceptor-adapter or at the 5′ end of the primer adapter. In some embodiments, the acceptor-adapter comprises a 3′-dideoxynucleotide.

In some embodiments, the hybrid reverse transcriptase comprises at least one improved property including, but not limited to, higher processivity, longer shelf life, higher strand displacement, higher end-to-end template jumping, and higher affinity as compared to a non-retroviral retrotransposon, or a fragment of the non-retroviral retrotransposon, that is not joined to a nucleic acid binding protein. In certain embodiments, the processivity of the hybrid reverse transcriptase is about 20 or more nucleotides.

### BRIEF DESCRIPTION OF THE DRAWINGS

FIGS. 1A and 1B illustrates hybrid reverse transcriptase constructs according to embodiments. In the embodiments, one or more nucleic acid binding proteins or one or more fragments of nucleic acid binding protein is recombinantly joined via an optional linker to an N-terminus (FIG. 1A) or a C-terminus (FIG. 1B) of a nonviral retrotransposon or fragment of the nonviral retrotransposon. In FIGS. 1A and 1B (and subsequent figures), “ΔNABP” refers to nucleic acid binding protein minus fragments that do not affect the function of the nucleic acid binding protein.

FIGS. 2A-4B illustrate methods of producing hybrid reverse transcriptases according to embodiments. In the embodiments, a nucleic acid binding protein, or a fragment of the nucleic acid binding protein, is joined via protein ligation to a nonviral retrotransposon or a fragment of the nonviral retrotransposon.

FIGS. 5A-5C illustrate methods of producing hybrid reverse transcriptases according to embodiments in which a first nonviral retrotransposon or a first fragment of the nonviral retrotransposon is joined via protein ligation to a second nonviral retrotransposon or a second fragment of the nonviral retrotransposon.

FIG. 6 illustrates a method of producing hybrid reverse transcriptase according to an embodiment. In the embodiment, a biotinylated nonviral retrotransposon or a biotinylated fragment of the nonviral retrotransposon and a biotinylated nucleic acid binding protein or a biotinylated fragment of the nucleic acid binding protein are joined to a biotin-binding protein via non-covalent interactions.

FIG. 7 illustrate a method of producing hybrid reverse transcriptase according to an embodiment in which a first biotinylated nonviral retrotransposon or a first biotinylated fragment of the nonviral retrotransposon and a second biotinylated nonviral retrotransposon or a second biotinylated fragment of the nonviral retrotransposon are joined to a biotin-binding protein via non-covalent interactions.

FIG. 8 illustrates a method of preparing a cDNA molecule using a hybrid reverse transcriptase comprising a dsDNA binding protein or a fragment of the dsDNA binding protein.

FIG. 9 illustrates a method of preparing a cDNA molecule library using a hybrid reverse transcriptase comprising a dsDNA binding protein or a fragment of the dsDNA binding protein. The method uses fragmented template RNA.

FIG. 10 illustrates a method of preparing a cDNA molecule library using a hybrid reverse transcriptase comprising a dsDNA binding protein or a fragment of the dsDNA binding protein. In the method, template RNA molecule end repair and poly A tailing steps are combined into one step.

FIG. 11 illustrates a method of removing adapter-dimer artifacts generated in methods of preparing cDNA molecules according to embodiments.

FIG. 12 illustrates a method of preparing a cDNA molecule library using a hybrid reverse transcriptase comprising a dsDNA binding protein or a fragment of the dsDNA binding protein. The method uses a messenger RNA template comprising a poly A tail and a primer-adapter comprising a poly T tail.

FIG. 13 illustrates a method of preparing a cDNA molecule library using a hybrid reverse transcriptase comprising a dsDNA binding protein or a fragment of the dsDNA binding protein. The method uses a messenger RNA template comprising a poly A tail and one or more random primer-adapters.

FIG. 14 illustrates a method of preparing a cDNA molecule using a hybrid reverse transcriptase comprising a ssDNA binding protein or a fragment of the ssDNA binding protein.

FIG. 15 illustrates a method of preparing a cDNA molecule using a hybrid reverse transcriptase comprising a ssDNA binding protein. The hybrid reverse transcriptase is a fusion protein. The ssDNA binding protein is an anti-Digoxigenin antibody and the ssDNA or ssRNA primer comprises a Digoxigenin tag.

FIG. 16 illustrates a method of preparing a cDNA molecule library using a hybrid reverse transcriptase comprising a ssDNA binding protein, or a fragment of the ssDNA binding protein, in which a primer-adapter is not complementary to the template RNA molecule. Template RNA molecule end repair and poly A tailing steps are absent.

FIG. 17 illustrates a method of preparing a cDNA molecule library using a hybrid reverse transcriptase comprising a ssDNA binding protein, or a fragment of the ssDNA binding protein, in which the primer-adapter is not complementary to the template RNA molecule. Template RNA molecule end repair and poly A tailing steps are absent. The method illustrated in FIG. 11 is used to remove adapter-dimer artifacts.

FIG. 18 illustrates a method of preparing a cDNA molecule library using a hybrid reverse transcriptase comprising a ssDNA binding protein, or a fragment of the ssDNA binding protein, in which a primer-adapter is not complementary to the template RNA molecule. Fragmentation, 3′ end priming with the primer-adapter, primer extension, and jumping of the hybrid reverse transcriptase to an acceptor-adapter occur in one step.

FIG. 19 illustrates a method of preparing a cDNA molecule using a hybrid reverse transcriptase comprising a ssDNA binding protein, or a fragment of the ssDNA binding protein, in which a primer-adapter is not complementary to the template RNA molecule. Fragmentation, 3′ end priming with the primer-adapter, primer extension, and jumping of the hybrid reverse transcriptase to an acceptor-adapter occur in a droplet. The method is used for single-cell transcriptomics.

FIG. 20 illustrates hybrid reverse transcriptase constructs used in expression studies as described in Example 1. ΔR2 enzyme is R2 enzyme minus the N-terminal DNA binding domain.

FIGS. 21A and 21B are images of SDS-PAGE gels of expressed hybrid reverse transcriptases in which a nucleic acid binding protein is joined to the N-terminus or C-terminus, respectively, of ΔR2 enzyme.

FIG. 22 is an image of an SDS-PAGE gel of a purified hybrid reverse transcriptase in which a dimer of a fragment of Sulfolobus SSB is joined to the C-terminus of ΔR2 enzyme.

### DETAILED DESCRIPTION

The invention provides hybrid reverse transcriptases that comprise a non-retroviral retrotransposon, or a fragment of the non-retroviral retrotransposon having reverse transcriptase activity, is covalently joined (i.e., via recombinant methods or via protein ligation) to a nucleic acid binding protein, or a fragment of the nucleic acid binding protein. The invention also provides hybrid reverse transcriptases that comprise a biotinylated non-retroviral retrotransposon, or a fragment of the biotinylated non-retroviral retrotransposon having reverse transcriptase activity, and a biotinylated nucleic acid binding protein, or a fragment of the biotinylated nucleic acid binding protein, non-covalently joined to a biotin-binding protein. The invention comprises nucleic acid constructs encoding the hybrid reverse transcriptases and vectors comprising the nucleic acid constructs. The hybrid reverse transcriptases can jump from a 5′ end of a first nucleic acid template to a 3′ end of a second nucleic acid template (an “acceptor-adapter”). Hybrid reverse transcriptases comprising a single-stranded nucleic acid binding protein, or a fragment of the single-stranded nucleic acid binding protein, can be used with primers that are not complementary to a template RNA. Hybrid reverse transcriptases comprising a ssDNA binding protein, or a fragment of the ssDNA binding protein can also be used with template RNA that is phosphorylated and does not have a poly (A) tail.

The hybrid reverse transcriptases may have the following improved properties: higher processivity (i.e., does not dissociate from the RNA template before reaching the 5′ end), higher strand displacement, higher end-to-end template jumping, higher affinity, and longer shelf life as compared to a non-retroviral retrotransposon, or a fragment of the non-retroviral retrotransposon, that is not joined to a nucleic acid binding protein. The hybrid reverse transcriptases are suitable for use in methods to generate cDNA libraries from total-RNA, messenger RNA (mRNA), micro RNA (miRNA), ribosomal RNAs, transfer RNAs (tRNAs), long non-coding RNA, cell free-RNA or from a single cell. The hybrid reverse transcriptases are also suitable for use in nucleic acid amplification methods (e.g., PCR, quantitative PCR (qPCR), reverse transcription PCR (RT-PCR), or digital droplet PCR (ddPCR)), and sequencing methods. Methods to generate a cDNA library can be performed in a partition (e.g., a droplet in an emulsion) or in a single reaction vessel.

### Hybrid Reverse Transcriptases

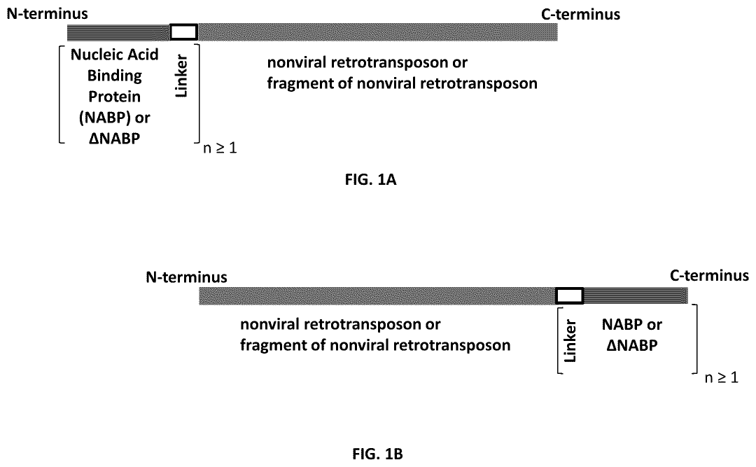

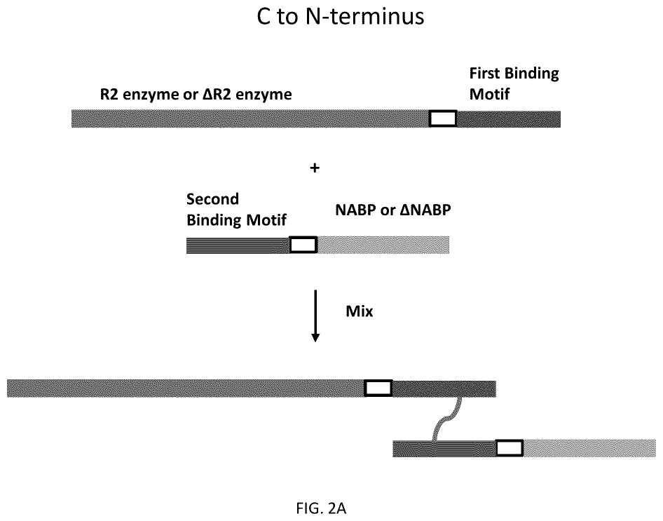

In an embodiment, a hybrid reverse transcriptase comprises a non-retroviral retrotransposon, or a fragment of the non-retroviral retrotransposon having reverse transcriptase activity, joined to a nucleic acid binding protein or a fragment of the nucleic acid binding protein (FIGS. 1A and 1B). In some embodiments, the hybrid reverse transcriptase is a fusion protein. As used herein, a “fusion protein” refers to a protein comprising two or more polypeptides attached by covalent bonding. The two or more polypeptides are encoded by separate genes that are joined so that they are transcribed and translated as a single unit (i.e., in a single open reading frame), producing a single polypeptide that has functional properties derived from each of the original polypeptides. Fusion of the nucleic acid binding protein, or a fragment of the nucleic acid binding protein, to a non-retroviral retrotransposon, or a fragment of the non-retroviral retrotransposon, may enhance the ability of the enzyme to bind and reverse transcribe the nucleic acid.

As used herein, “non-retroviral retrotransposon” refers to naturally occurring proteins encoded by non-retroviral retrotransposons and polypeptide fragments of non-retroviral retrotransposons having reverse transcriptase activity, as well as proteins or polypeptides derived therefrom having one or more amino acid substitutions that either enhance the reverse transcriptase activity thereof or have no deleterious effect thereon. A class of non-retroviral retrotransposon is R2 proteins or polypeptides. Thus, as used herein, “R2 protein or R2 enzyme or polypeptide or a functional fragment thereof” refers to naturally occurring proteins encoded by R2 elements and polypeptide fragments thereof which possess reverse transcriptase activity, as well as proteins or polypeptides derived therefrom which contain one or more amino acid substitutions that either enhance the reverse transcriptase activity thereof or have no deleterious effect thereon.

In some embodiments, the non-retroviral retrotransposon, or the fragment of the non-retroviral retrotransposon is a non-long terminal repeat (LTR) retrotransposon derived from the R2 element of the silkmoth, Bombyx mori. In an embodiment, the non-retroviral retrotransposon comprises at least 60%, 65%, 70%, 75%, 80%, 85%, 90%, 91%, 92%, 93%, 94%, 95%, 96%, 97%, 98%, or 99% sequence identity to SEQ ID NO: 1 (i.e., wild type R2 enzyme derived from Bombyx mori).

The R2 element of many arthropods have three functional domains: an N-terminal DNA binding domain, a central reverse transcriptase domain, and a C-terminal endonuclease domain (Burke et al., “The Domain Structure and Retrotransposition Mechanism of R2 Elements Are Conserved Throughout Arthropods,” Mol. Biol. Evol. 16(4):502-511 (1999)). In certain embodiments, the fragment derived from the R2 element of the silkmoth is lacking an N-terminal DNA binding domain. In some embodiments, the fragment derived from the R2 element of the silkmoth has at least 60%, 65%, 70%, 75%, 80%, 85%, 90%, 91%, 92%, 93%, 94%, 95%, 96%, 97%, 98%, or 99% sequence identity to SEQ ID NO: 2.

Proteins derived from the R2 element of other arthropods than the silkmoth can also be used in embodiments of the hybrid reverse transcriptase and include, but are not limited to, R2 elements from Drosophila spp. (fruit fly), Forficula auricularia (earwig), Popillia japonica (Japanese beetle), Nasonia vitripennis (jewel wasp), Tenebrio molitor (mealworm), Collembola spp. (springtails), Isopoda spp. (pillbugs), and Limulus polyphemus (horseshoe crab). Any of the proteins listed in Table 1, including homologs, variants, or fragments thereof of the proteins, can also be used in the hybrid reverse transcriptase. The proteins listed in Table 1 are from the same phylogenetic tree as silkmoth and have at least 27% identity to silkmoth. In some embodiments, the hybrid reverse transcriptase comprises R2 element domains from two or more arthropods. For example, the N-terminal binding domain from any of the proteins listed in Table 1 can be combined with the central reverse transcriptase domain and the C-terminal endonuclease domain of the silk worm.

TABLE 1

| GenBank | |
|---|---|
| Number | Description |
| KRX33447.1 | retrovirus-related Pol polyprotein from type-2 |
| | retrotransposable element R2DM, partial |
| | [Trichinella murrelli] |
| KRX36111.1 | retrovirus-related Pol polyprotein from type-2 |
| | retrotransposable element R2DM [Trichinella murrelli] |
| KRZ48391.1 | Retrovirus-related Pol polyprotein from type-1 |
| | retrotransposable element [Trichinella nativa] |
| KRZ66264.1 | Retrovirus-related Pol polyprotein from type-2 |
| | retrotransposable element R2DM [Trichinella papuae] |
| BAC82594.1 | reverse transcriptase [Danio rerio] |
| AAC34903.1 | reverse transcriptase, partial [Anurida maritima] |
| AAC34904.1 | reverse transcriptase, partial [Limulus polyphemus] |
| AFO19998.1 | R2 protein [Lepidurus couesii] |
| KMQ90176.1 | Reverse transcriptase [Lasius niger] |
| ACJ71597.1 | reverse transcriptase [Rhynchosciara americana] |
| AIL01110.1 | Reverse transcriptase [Bacillus rossius] |
| KMQ88340.1 | reverse transcriptase [Lasius niger] |
| AFM44926.1 | R2 protein [Eyprepocnemis plorans] |
| AAB94032.1 | reverse transcripta [Drosophila mercatorum] |
| KRY44798.1 | Retrovirus-related Pol polyprotein from type-2 |
| | retrotransposable element R2DM [Trichinella britovi] |
| CAX83712.1 | endonuclease-reverse transcriptase [Schistosoma |
| | japonicum] |
| PIS84891.1 | reverse transcriptase [Fasciola hepatica] |
| BAX24491.1 | reverse transcriptase [Beryx splendens] |
| ACV95454.1 | reverse transcriptase/endonuclease [Adineta vaga] |
| PAA70689.1 | hypothetical protein BOX15_Mlig021084g2 |
| | [Macrostomum lignano] |

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

Sequences are “substantially identical” to each other if they have a specified percentage of nucleotides or amino acid residues that are the same (e.g., at least 60%, 65%, 70%, 75%, 80%, 85%, 90%, 91%, 92%, 93%, 94%, 95%, 96%, 97%, 98%, or 99% identity over a specified region or the entire designated sequence if a region is not specified), when compared and aligned for maximum correspondence over a comparison window.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, about 10 to about 300, about 10 to about 150 in which a sequence may be compared to a reference sequence of the same number of contiguous positions after the two sequences are optimally aligned. The comparison window can also be the entire length of either the reference or the test sequence.

Percent sequence identity and sequence similarity can be determined using the BLAST 2.0 algorithm, which is described in Altschul et al. (J. Mol. Biol. 215:403-10, 1990). Software for performing BLAST 2.0 analyses is publicly available through the National Center for Biotechnology Information (Worldwide Website: ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The nucleic acid binding protein portion of the hybrid reverse transcriptase binds to nucleic acid in a nucleotide sequence-independent manner, (i.e., binding does not exhibit a preference for a particular sequence). In certain embodiments, the nucleic acid binding protein is a double-stranded DNA (dsDNA) binding protein (i.e., the dsDNA binding protein preferentially binds dsDNA over single-stranded DNA (ssDNA)), or a ssDNA binding protein. In some embodiments, a dsDNA binding protein is Sso7d (SEQ ID NO: 3) or Cren7 (SEQ ID NO: 4) or homologs, variants, or fragments thereof. Any of the dsDNA binding proteins listed in TABLES 2 and 3, including homologs, variants, or fragments thereof, can also be used in the hybrid reverse transcriptases.

TABLE 2

| UniProt Number | Description |
|---|---|
| P13123 | Sac7d [Sulfolobus acidocaldarius] |
| P13125 | Sac7e [Sulfolobus acidocaldarius] |
| A4YEA2 | Mse7 [Metallosphaera sedula] |
| F4FYY6 | Mcu7 [Metallosphaera cuprina] |
| F4B991 | Aho7a [Acidianus hospitalis] |
| F4B9I5 | Aho7b [Acidianus hospitalis] |
| F4B8X5 | Aho7c [Acidianus hospitalis] |
| Q96X56 | Sto7 [Sulfurisphaera tokodaii] |
| O59632 | Ssh7b [Saccharolobus shibatae] |
| D2PHL8 | Sis7a [Sulfolobus islandicus] |
| F0NJT3 | Sis7b [Sulfolobus islandicus] |
| P61990 | Ssh7a [S. shibatea] |
| P39476 | Sso7d [Saccharolobus Solfataricus] |

TABLE 3

| GenBank | |
|---|---|
| Number | Description |
| ARM76599.1 | chorismate-binding protein [Acidianus manzaensis] |
| WP_110270921.1 | chorismate-binding protein [Acidianus brierleyi] |
| WP_011822047.1 | chorismate-binding protein [Hyperthermus butylicus] |
| WP_058370641.1 | chorismate-binding protein [Pyrodictium occultum] |
| WP_109160791.1 | chorismate-binding protein [Desulfurococcaceae |
| | archaeon] |
| AFH42781.1 | chromatin protein Cren7 [Fervidicoccus fontis |
| | Kam940] |
| WP_014027454.1 | chorismate-binding protein [Pyrolobus fumarii] |

In some embodiments, a ssDNA binding protein is Sulfolobus SSB (SEQ ID NO: 5), or homologs, variants, or fragments thereof. In some embodiments, the ssDNA binding protein is anti-nucleic acid tag antibody (e.g., anti-Digoxigenin antibody). Any of the ssDNA binding proteins listed in TABLE 4, including homologs, variants, or fragments thereof, can also be used in the hybrid reverse transcriptases.

TABLE 4

| GenBank Number | Description |
|---|---|
| WP_013561922.1 | single-stranded DNA-binding protein |
| | [Desulfurococcus mucosus] |
| PVU72461.1 | single-stranded DNA-binding protein |
| | [Sulfolobales archaeon SCGC AB-777_J03]) |
| WP_013561922.1 | single-stranded DNA-binding protein |
| | [Thermoplasma sp. Kam2015] |
| RLG83789.1 | single-stranded DNA-binding protein |
| | [Thermoprotei archaeon] |
| ALU11621.1 | single-stranded DNA-binding protein |
| | [Ignicoccus islandicus DSM 13165] |
| WP_012718085.1 | single-stranded DNA-binding protein |
| | [Sulfolobus islandicus] |
| WP_010978480.1 | single-stranded DNA-binding protein |
| | [Sulfurisphaera tokodaii] |
| WP_012020631.1 | single-stranded DNA-binding protein |
| | [Metallosphaera sedula] |
| EWG07842.1 | single-stranded DNA-binding protein |
| | [Candidatus Aramenus sulfurataquae] |
| RLG38958.1 | single-stranded DNA-binding protein |
| | [Candidatus Korarchaeota archaeon] |
| WP_011821997.1 | single-stranded DNA-binding protein |
| | [Hyperthermus butylicus] |
| BAN90306.1 | ssDNA-binding protein [Aeropyrum camini |
| | SY1 = JCM 12091] |
| WP_131159968.1 | single-stranded DNA-binding protein |
| | [Aeropyrum pernix] |
| BAA80315.2 | single-stranded DNA binding protein |
| | [Aeropyrum pernix K1] |
| TDA40126.1 | single-stranded DNA-binding protein |
| | [Candidatus Verstraetearchaeota archaeon] |
| RLI67762.1 | hypothetical protein DRO63_04010 |
| | [Candidatus Heimdallarchaeota archaeon] |
| OLD91798.1 | hypothetical protein AUG86_00240 |
| | [Euryarchaeota archaeon 13_1_20CM_4_64_14] |
| WP_010901557.1 | single-stranded DNA-binding protein |
| | [Thermoplasma acidophilum] |
| PUA31887.1 | hypothetical protein B9J98_05180 |
| | [Aigarchaeota archaeon NZ13_MG1] |
| RLG96403.1 | hypothetical protein DRO27_02395 |
| | [Candidatus Bathyarchaeota archaeon] |
| WP_054838323.1 | single-stranded DNA-binding protein |
| | [Sulfolobus metallicus] |
| PMP92383.1 | single-stranded DNA-binding protein |
| | [Caldisphaera sp.] |
| EQB65396.1 | hypothetical protein AMDU3_IPLC00002G0033 |
| | [Thermoplasmatales archaeon I-plasma] |
| WP_055409701.1 | single-stranded DNA-binding protein |
| | [Pyrodictium delaneyi] |
| WP_100692737 | single-stranded DNA-binding protein SSB1 |
| | [Escherichia coli] |
| WP_003898320 | single-stranded DNA-binding protein |
| | [Mycolicibacterium smegmatis] |

In some embodiments, one or more nucleic acid binding proteins, or one or more fragments of a nucleic acid binding protein, is joined to the non-retroviral retrotransposon, or the fragment of the non-retroviral retrotransposon (FIGS. 1A and 1B). The nucleic acid binding proteins or fragments thereof can be identical or non-identical and can be joined in sequential or random order to the N-terminus (FIG. 1A) or the C-terminus (FIG. 1B) of the non-retroviral retrotransposon, or the fragment of the non-retroviral retrotransposon.

In some embodiments, the hybrid reverse transcriptase comprises at least one linker between the non-retroviral retrotransposon, or the fragment of the non-retroviral retrotransposon, and the nucleic acid binding protein, or a fragment of the nucleic acid binding protein. As used herein, a “linker” refers to a peptide or polypeptide containing one or more amino acid residues (e.g., 1, 2, 3, 4, 5, 10 or more amino acid residues) joined by a peptide bond(s). Such linkers can provide rotational freedom that allows each component of the hybrid reverse transcriptase to interact with its intended target without hindrance. These linkers can be mixtures of glycine and serine, such as -(GGS)ₙ-, -(GGGS)ₙ- (SEQ ID NO: 108), -(GGGGS)ₙ-(SEQ ID NO: 109), and -(EAAAK)ₙ- (SEQ ID NO: 110) where n is 1, 2, 3, 4, or 5 where n is any integer. Other suitable peptide/polypeptide linker sequences optionally include naturally occurring or non-naturally occurring peptides or polypeptides. Optionally, the peptide or polypeptide linker sequences are flexible peptides or polypeptides. Exemplary flexible peptides/polypeptides include, but are not limited to, the amino acid sequences Gly-Ser, Gly-Ser-Gly-Ser (SEQ ID NO: 118), Ala-Ser, Gly-Gly-Gly-Ser (SEQ ID NO: 108), Gly₄-Ser (SEQ ID NO: 109), (Gly₄-Ser)₂, (Gly₄-Ser)₃, (Gly₄-Ser)₄, (Gly₄-Ser)₂-Gly-Ala-Gly-Ser-Gly₄-Ser (SEQ ID NO: 119), Gly-(Gly₄-Ser)₂ (SEQ ID NO: 120), Gly₄-Ser-Gly (SEQ ID NO: 121), Gly-Ser-Gly₂ (SEQ ID NO: 122), and Gly-Ser-Gly₂-Ser (SEQ ID NO: 123). Exemplary peptides/polypeptides include, but are not limited to, VGTVGTGGGSGGASTAL (SEQ ID NO: 101), VGTVGTGGGSEAAAKGGASTAL (SEQ ID NO: 102), VGTGGGSEAAAKGGASTAL (SEQ ID NO: 103), VGTGGGSGGGEAAAKEAAAKSGGGS (SEQ ID NO: 104), VGTGGGSGGGEAAAKEAAAKSGGGSA (SEQ ID NO: 105), VGTGGGSGGGTGGGS (SEQ ID NO: 106), and VGTGGGSGGGTGGGSA (SEQ ID NO: 107). In some embodiments, a sequence of 1 to 50 amino acid residues can be used as a linker.

In some embodiments, a hybrid reverse transcriptase comprises two or more non-retroviral retrotransposons, or two or more fragments of the non-retroviral retrotransposon having reverse transcriptase activity, joined by an optional linker. In some embodiments, the hybrid reverse transcriptase comprises two or more fragments of R2 enzyme joined by an optional linker.

Hybrid reverse transcriptase constructs according to embodiments are listed in TABLE 5 along with their respective SEQ ID NO. In TABLE 5, ORF refers to Original Reading Frame. “WT R2 enzyme” refers to wild type R2 enzyme (SEQ ID NO: 1). “ΔR2 enzyme” refers to R2 enzyme minus the N-terminal DNA binding domain (SEQ ID NO: 2). “ΔSSB” refers to SSB minus C-terminal fragments (SEQ ID NO: 46).

TABLE 5

| SEQ ID | | Linker | |
|---|---|---|---|
| NO: | N-terminal ORF | ORF | C-terminal ORF |
| 6 and 7 | Sso7d | Linker | WT R2 enzyme |
| | | | and ΔR2 enzyme |
| 8 and 9 | Sso7d-Sso7d | Linker | WT R2 enzyme |
| | | | and ΔR2 enzyme |
| 10 and 11 | Cren7 | Linker | WT R2 enzyme |
| | | | and ΔR2 enzyme |
| 12 and 13 | Cren7-Cren7 | Linker | WT R2 enzyme |
| | | | and ΔR2 enzyme |
| 14 and 15 | SSB | Linker | WT R2 enzyme |
| | | | and ΔR2 enzyme |
| 16 and 17 | SSB-SSB | Linker | WT R2 enzyme |
| | | | and ΔR2 enzyme |
| 18 and 19 | WT R2 enzyme | Linker | Sso7d |
| | and ΔR2 enzyme | | |
| 20 and 21 | WT R2 enzyme | Linker | Sso7d-Sso7d |
| | and ΔR2 enzyme | | |
| 22 and 23 | WT R2 enzyme | Linker | Cren7 |
| | and ΔR2 enzyme | | |
| 24 and 25 | WT R2 enzyme | Linker | Cren7-Cren7 |
| | and ΔR2 enzyme | | |
| 26 and 27 | WT R2 enzyme | Linker | Cren7-Cren7-Cren7 |
| | and ΔR2 enzyme | | |
| 28 and 29 | WT R2 enzyme | Linker | Sso7d-Cren7 |
| | and ΔR2 enzyme | | |
| 30 and 31 | WT R2 enzyme | Linker | Sso7d-Cren7-Cren7 |
| | and ΔR2 enzyme | | |
| 32 and 33 | WT R2 enzyme | Linker | Cren7-Sso7d |
| | and ΔR2 enzyme | | |
| 34 and 35 | WT R2 enzyme | Linker | SSB |
| | and ΔR2 enzyme | | |
| 36 and 37 | WT R2 enzyme | Linker | SSB-SSB |
| | and ΔR2 enzyme | | |
| 38 and 39 | WT R2 enzyme | Linker | SSB-SSB-SSB |
| | and ΔR2 enzyme | | |
| 40 and 41 | WT R2 enzyme | Linker | SSB-Cren7 |
| | and ΔR2 enzyme | | |
| 42 and 43 | WT R2 enzyme | Linker | SSB-Cren7-Cren7 |
| | and ΔR2 enzyme | | |
| 44 and 45 | WT R2 enzyme | Linker | WT R2 enzyme |
| | and ΔR2 enzyme | | and ΔR2 enzyme |
| 98 and 47 | WT R2 enzyme | Linker | ΔSSB-ΔSSB |
| | and ΔR2 enzyme | | |
| 99 and 100 | ΔSSB-ΔSSB | Linker | WT R2 enzyme |
| | | | and ΔR2 enzyme |

Accordingly, in some embodiments, the hybrid reverse transcriptase comprises a sequence with at least 75% identity to SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 98, SEQ ID NO: 99, or SEQ ID NO: 100. In some embodiments, the hybrid reverse transcriptase comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 98, SEQ ID NO: 99, or SEQ ID NO: 100.

### Hybrid Reverse Transcriptases Formed Via Protein Ligation

The term “binding motif” refers to a protein sequence that is attached to a first polypeptide (e.g., a non-retroviral retrotransposon or a fragment of the non-retroviral retrotransposon having reverse transcriptase activity) and a second polypeptide (e.g., a nucleic acid binding protein/fragment of the nucleic acid binding protein or a second non-retroviral retrotransposon/fragment of the non-retroviral retrotransposon). The binding motifs facilitate the formation of a covalent bond between the first and second polypeptides. Non-limiting examples of binding motifs include SpyTag sequences (including SpyTag002), SpyCatcher sequences (including SpyCatcher short and SpyCatcher002), SnoopTag sequences (including SnoopTagJr), SnoopCatcher sequences, Isopeptag sequences, Split Spy0128 sequences, K-tag sequences, SdyTag sequences, SdyCatcherDANG short sequences, DogTag sequences, and sortase sequences (including sortase recognition domain and sortase bridging domain). The binding motifs may be fused to an N-terminus or a C-terminus of the polypeptide. One or more linker sequences (e.g., a glycine/serine rich linker) may flank the binding motifs to enhance accessibility for reaction or to enhance flexibility of the fused polypeptides.

The term “protein ligation” as used herein refers to site-specific covalent bond formation, either spontaneously or with the help of an enzyme, between a first binding motif and a second binding motif when the first and second binding motifs are brought into contact with one another. Non-limiting examples of the protein ligation systems include the SpyTag/SpyCatcher system, SpyTag with the shorter version of SpyCatcher (i.e., SpyCatcher short), SpyTag002 and SpyCatcher002 with accelerated reaction, the SpyTag/K-tag/SpyLigase system, the Isopeptag/Split Spy0128 system, the SnoopTag/SnoopCatcher system, the SdyTag/SdyCatcher system, the SnoopTagJr/DogTag/SnoopLigase system, and the sortase system.

Protein ligation occurs between mutually reactive or cognate pairs of binding motifs. For example, if the first binding motif is SpyTag (SEQ ID NO: 48), then the second binding motif can be SpyCatcher (SEQ ID NO: 51), SpyCatcher002 (SEQ ID NO: 52), or SpyCatcher short (SEQ ID NO: 53), but cannot be SnoopCatcher (SEQ ID NO: 59) or SdyCatcherDANG short (SEQ ID NO: 60) because the SpyTag/SpyCatcher system is orthogonal to the SnoopTag/SnoopCatcher system. As used herein, “orthogonal” refers to mutually unreactive or noncognate binding pairs. Protein ligation can also occur between the following combinations of binding motifs: between SpyTag or SpyTag002 (SEQ ID NO: 49) and SpyCatcher, SpyCatcher short, or SpyCatcher002 (SEQ ID NO: 52); between SnoopTag (SEQ ID NO: 56) and SnoopCatcher (SEQ ID NO: 59); between Isopeptag (SEQ ID NO: 50) and Split Spy0128 (SEQ ID NO: 54); between SdyTag (SEQ ID NO: 57) and SdyCatcherDANG short (SEQ ID NO: 60), between SpyTag and K-Tag (SEQ ID NO: 55); and between SnoopTagJr (SEQ ID NO: 58) and DogTag (SEQ ID NO: 61).

The sortase system uses sortase enzymes and sortase recognition and bridging domains. In embodiments of the hybrid reverse transcriptase, the sortase recognition and bridging domains are considered binding motifs. Sortases are transpeptidases produced by Gram-positive bacteria to anchor cell surface proteins covalently to the cell wall. The Staphylococcus aureus sortase A (SrtA) cleaves a short C-terminal recognition motif (LPXTG (SEQ ID NO: 66) (referred to herein as a sortase recognition domain). The sortase recognition domain is a sortase A recognition domain or a sortase B recognition domain. The sortase A recognition domain comprises or consists of the amino acid sequence: LPTGAA (SEQ ID NO: 62), LPTGGG (SEQ ID NO: 63), LPKTGG (SEQ ID NO: 64), LPETG (SEQ ID NO: 65), LPXTG (SEQ ID NO: 66) or LPXTG(X)ₙ (SEQ ID NO: 67), where X is any amino acid, and n is 0, 1, 2, 3, 4, 5, 7, 8, 9, 10, in the range of 0-5 or 0-10, or any integer up to 100. The sortase B recognition domain comprises the amino acid sequence NPX1TX2 (SEQ ID NO: 68), where X1 is glutamine or lysine; X2 is asparagine or glycine; N is asparagine; P is proline and T is threonine.

The sortase A and B bridging domains comprise one or more glycine residues at the N-terminus of a peptide. In certain embodiments, the one or more glycine residues may optionally be: Gly, (Gly)₂, (Gly)₃, (Gly)₄, or (Gly)ₓ, where x is an integer of 1-20.

Components of the mutually reactive binding motif pair or cognate binding motif pair can be interchanged between the non-retroviral retrotransposon, or the fragment of the non-retroviral retrotransposon, and the nucleic acid binding protein, or a fragment of the nucleic acid binding protein (e.g., one embodiment provides a non-retroviral retrotransposon, or a fragment of the non-retroviral retrotransposon, comprising SpyTag and a nucleic acid binding protein, or a fragment of the nucleic acid binding protein, comprising SpyCatcher, SpyCatcher short, or SpyCatcher002. An alternative embodiment provides a nucleic acid binding protein, or a fragment of the nucleic acid binding protein, comprising SpyTag and a non-retroviral retrotransposon, or a fragment of the non-retroviral retrotransposon, comprising SpyCatcher, SpyCatcher short, or SpyCatcher002). Exemplary first and second binding motifs for the hybrid reverse transcriptase embodiments are provided in Table 5.

TABLE 6

| First Binding Motif | Second Binding Motif |
|---|---|
| SEQ ID NO: 48, 49, or 50 or a | SEQ ID NO: 51, 52, 53, 54, or 55 or |
| sequence with at least 60% | a sequence with at least 60% |
| sequence identity to SEQ ID | sequence identity to SEQ ID |
| NO: 48, 49, or 50. | NO: 51, 52, 53, 54, or 55. |
| SEQ ID NO: 51, 52, 53, 54, or 55 | SEQ ID NO: 48, 49, or 50 or a |
| or a sequence with at least 60% | sequence with at least 60% sequence |
| sequence identity to SEQ ID NO: | identity to SEQ ID NO: 48, 49, or |
| 51, 52, 53, 54, or 55. | 50. |
| SEQ ID NO: 56, 57, or 58 or a | SEQ ID NO: 59, 60, or 61 or a |
| sequence with at least 60% | sequence with at least 60% sequence |
| sequence identity to SEQ ID | identity to SEQ ID NO: 59, 60, or |
| NO: 56, 57, or 58. | 61. |
| SEQ ID NO: 59, 60, or 61 or a | SEQ ID NO: 56, 57, or 58 or a |
| sequence with at least 60% | sequence with at least 60% sequence |
| sequence identity to SEQ ID NO: | identity to SEQ ID NO: 56, 57, or |
| 59, 60, or 61. | 58. |
| SEQ ID NO: 62, 63, 64, 65, 66, | Gly, (Gly)₂, (Gly)₃, (Gly)₄, or |
| 67, or 68. | (Gly)ₓ, where x is an integer of 1-20 |
| Gly, (Gly)₂, (Gly)₃, (Gly)₄, or | SEQ ID NO: 62, 63, 64, 65, 66, 67, |
| (Gly)ₓ, where x is an integer of | or 68. |
| 1-20 | |

In some embodiments, the binding motif is joined to the N- or C-terminus of the non-retroviral retrotransposon, the fragment of the non-retroviral retrotransposon, the nucleic acid binding protein, or a fragment of the nucleic acid binding protein, via an optional linker as indicated in Table 7. In Table 7, ORF refers to Original Reading Frame. “WT R2 enzyme” refers to wild type R2 enzyme (SEQ ID NO: 1). “ΔR2 enzyme” refers to R2 enzyme minus the N-terminal DNA binding domain (SEQ ID NO: 2). “ΔNucleic Acid Binding Protein” refers to nucleic acid binding protein minus a fragment(s).

TABLE 7

| | Linker | |
|---|---|---|
| N-terminal ORF | ORF | C-terminal ORF |
| WT R2 enzyme or ΔR2 enzyme | Linker | First or Second Binding Motif |
| Nucleic Acid Binding Protein | Linker | First or Second Binding Motif |
| (Sso7d, Cren7, or SSB) or | | |
| ΔNucleic Acid Binding Protein | | |
| First or Second Binding Motif | Linker | WT R2 enzyme or ΔR2 enzyme |
| First or Second Binding Motif | Linker | Nucleic Acid Binding Protein |
| | | (Sso7d, Cren7, or SSB) or |
| | | ΔNucleic Acid Binding Protein |

Hybrid RTs Formed Via Non-Covalent Process with AviTag

The term “acceptor peptide” refers to an amino acid sequence that is attached to a first polypeptide (e.g., a non-retroviral retrotransposon or a fragment of the non-retroviral retrotransposon having reverse transcriptase activity) and a second polypeptide (e.g., a nucleic acid binding protein or a second non-retroviral retrotransposon/fragment of the non-retroviral retrotransposon). The acceptor peptide is a substrate for enzymatic biotinylation. As used herein, “enzymatic biotinylation” refers to the enzyme-catalyzed attachment of biotin to a lysine group in the acceptor peptide. The acceptor peptide can be biotinylated in vitro or in vivo by, for example, E. coli biotin ligase (the BirA protein) in the presence of biotin and ATP (Beckett et al., 1999). Non-limiting examples of acceptor peptides include, but are not limited to AviTag (SEQ ID NO: 85; GLNDIFEAQKIEWHE), BioTag (SEQ ID NO: 86; ALNDIFEAQKIEWHA), Biotin ligase recognition peptide (SEQ ID NO: 87; MAGGLNDIFEAQKIEWHEDTGGS), BirA Substrate Peptide (SEQ ID NO: 88; LHHILDAQKMVWNHR) and LX§ IFEAQKIEWR (SEQ ID NO: 89), where X=any amino acid and § =any amino acid but not L, V, I, W, F or Y (Fairfield et al., 2015). Beckett et al. (1999) and Fairfield et al. (2015) are hereby incorporated by reference in their entirety.

The acceptor peptide may be fused to the N- or C-terminus of the non-retroviral retrotransposon, the fragment of the non-retroviral retrotransposon, or the nucleic acid binding protein via an optional linker as indicated in Table 8.

TABLE 8

| | Linker | |
|---|---|---|
| N-terminal ORF | ORF | C-terminal ORF |
| WT R2 enzyme or ΔR2 enzyme | Linker | Acceptor Peptide |
| Nucleic Acid Binding Protein | Linker | Acceptor Peptide |
| (Sso7d, Cren7, or SSB) or | | |
| ΔNucleic Acid Binding Protein | | |
| Acceptor Peptide | Linker | WT R2 enzyme or ΔR2 enzyme |
| Acceptor Peptide | Linker | Nucleic Acid Binding Protein |
| | | (Sso7d, Cren7, or SSB) or |
| | | ΔNucleic Acid Binding Protein |

As used herein, the term “non-covalent interactions” refers to interactions between molecules that do not involve the sharing of electrons. Examples of non-covalent interactions can include hydrogen bonding, electrostatic interactions, pi stacking, van der Waals interactions, and dipole-dipole interactions.

As used herein, the term “biotin-binding protein” refers to a protein that can specifically bind to biotin with high binding affinity. The biotin-binding protein can be streptavidin, traptavidin, or neutravidin or any protein that can bind to biotin with a binding affinity similar to streptavidin. In some embodiments, the dissociation constant of the biotin-binding protein is about 10⁻¹⁴ to about 10⁻¹⁵ mol/L.

In certain embodiments, a purification tag is attached to the N-terminus, the C-terminus, or to both the N- and C-termini of the hybrid reverse transcriptase. Exemplary purification tags include, but are not limited to, polyhistidine or His-tag and FLAG®-tag (i.e., amino acid sequence DYKDDDDK (SEQ ID NO: 124) where D is aspartic acid, Y is tyrosine, and K is lysine).

In certain embodiments, the hybrid reverse transcriptase comprises at least one improved property as compared to a reverse transcriptase that does not comprise a nucleic acid binding domain (i.e., a non-retroviral retrotransposon, or a fragment of the non-retroviral retrotransposon having reverse transcriptase activity that is not joined to a nucleic acid binding protein). In some embodiments, the improved property is higher processivity, higher strand displacement, higher affinity, higher end-to-end template jumping, longer shelf life, and/or higher expression yield (i.e., higher milligrams fusion recovered per milligrams of E. coli cells). As used herein, “processivity” refers to the ability of a nucleic acid modifying enzyme to remain attached to the template or substrate and perform multiple modification reactions. Typically processivity refers to the ability to modify relatively long tracts of nucleic acid. In some embodiments, the processivity of the hybrid reverse transcriptase is about 20, 30, 40, 50 or more nucleotides.

As used herein, “strand displacement” is the ability of the fusion enzyme to displace a complementary strand of RNA or DNA during transcription, resulting in the ability of the enzyme to generate long cDNA products. As used herein, “affinity” is the ability of the fusion enzyme to bind and stay bound to the RNA template during transcription and; thus, is also related to the ability of the enzyme to modify relatively long tracts of nucleic acid. Since processivity, strand displacement, and affinity are related to the ability of the enzyme to transcribe long tracts of nucleic acid, they can be determined by, for example, using qPCR to quantify the amount or yield of cDNA products for specific templates. Exemplary templates that can be used are an External RNA Controls Consortium (ERCC) standard mix of 90 different RNA templates with known concentrations, lengths, and secondary structure (or GC content). Highly processive and strand displacing enzymes with high affinity will generate exact cDNA copies of the RNA templates with the original ratio of long to short RNA templates or high to low secondary structure. Low processive and strand displacing enzymes with low affinity will predominantly copy short RNA templates having low amounts of secondary structure. The ratio of amount of long to short transcripts and/or high to low secondary structure-containing transcripts can be measured with qPCR using appropriate primers for the RNA templates.

As used herein, “end-to-end template jumping” refers to the fusion enzyme jumping from a 3′ end of a primer-adapter to the 3′ end of the RNA template or from the 5′ end of the RNA template to a 3′ end of an acceptor-adapter. In some embodiments, end-to-end template jumping is measured by allowing the fusion enzyme to transcribe a short synthetic RNA template with an annealed DNA primer in the presence of a DNA acceptor-adapter and then determining a jumping efficiency. Jumping efficiency is the ratio (or percent) of fully reverse transcribed RNA template to jumping product (i.e., cDNA including sequences of RNA template and acceptor-adapter).

As used herein, a “primer” refers to a polynucleotide sequence that serves as a point of initiation of nucleic acid synthesis. The primer may optionally hybridize (i.e., is complementary) to a sequence on a target nucleic acid. Primers can be of a variety of lengths and are often less than 50 nucleotides in length, for example 12-30 nucleotides, in length. In some embodiments, a primer may comprise one or more random primer(s). In some embodiments, the primer is a “primer-adapter”. As used herein, a primer-adapter refers to a primer comprising an adapter sequence in which the 5′ tail sequence does not hybridize to the template nucleic acid. In some embodiments, the primer-adapter comprises a digoxigenin-tag (DIG-tag) label (i.e., digoxigenin is incorporated in the primer-adapter during synthesis).

As used herein, the term “acceptor-adapter” refers to a single-stranded nucleic acid to which a hybrid reverse transcriptase can jump from a 5′ end of a template nucleic acid in the absence of sequence identity between the accepter-adapter and the template nucleic acid. In some embodiments, an acceptor-adapter may be modified. In some embodiments, an acceptor-adapter may be modified at the 3′ end, for example to protect it from being mistaken as an RNA primer. In some embodiments, the modification of the acceptor-adapter comprises a dideoxy 3′ end. In some embodiments, the 3′ end of the acceptor-adapter is blocked by the addition of, for example, a C3 spacer, a phosphate, an amine group (NH₂), or any other chemical modification that inhibits formation of a subsequent phosphodiester bond between the 3′ end of the acceptor-adapter and another nucleotide. In certain embodiments, a 5′ end of the accepter-adapter comprises a nucleotide analogue that stops the reverse transcription.

As used herein, “shelf life” refers to the ability of the fusion enzyme to retain reverse transcriptase activity over a given period of time and at a given condition. In some embodiments, the fusion enzyme has a shelf life in solution at room temperature, −20° C., or 37° C. for at least 1 day (5 days, 10 days, 15 days, 20 days, 25 days, one month, 2 months, or 3 months). Shelf life can be determined by, for example, using qPCR and three or more reference RNA templates (e.g., ERCC RNAs, known synthetic RNAs, or Universal Human Reference RNAs) at known concentrations to quantify the amount of primer extension products and to determine when the fusion enzyme has lost 50% reverse transcriptase activity (i.e., when the amount of primer extension products has decreased by 50%). Thus, for example, if the fusion enzyme has lost 40% reverse transcriptase activity at 4 days in solution at room temperature and has lost 50% reverse transcriptase activity at 5 days in solution at room temperature, then the fusion enzyme has a shelf life of 5 days in solution at room temperature.

### Nucleic Acid Constructs

Also provided are the following nucleic acid constructs:
1. Nucleic acid constructs that encode for a hybrid reverse transcriptase, without or with a linker between the non-retroviral retrotransposon, or the fragment of the non-retroviral retrotransposon having reverse transcriptase activity, and the nucleic acid binding protein(s) or fragment(s) of the nucleic acid binding protein;
2. Nucleic acid constructs that encode for a non-retroviral retrotransposon, or a fragment of the non-retroviral retrotransposon having reverse transcriptase activity, and a nucleic acid binding protein, or a fragment of the nucleic acid binding protein, fused to a binding motif; and
3. Nucleic acid constructs that encode for a non-retroviral retrotransposon, a fragment of the non-retroviral retrotransposon having reverse transcriptase activity, and a nucleic acid binding protein, or a fragment of the nucleic acid binding protein, fused to an acceptor peptide.

Such nucleic acids can be present in an expression vector in an appropriate prokaryotic host cell.

In an embodiment, a pair of nucleic acid constructs comprises:
a) a first nucleic acid construct comprising a polynucleotide sequence encoding a non-retroviral retrotransposon, or a fragment of the non-retroviral retrotransposon having reverse transcriptase activity, fused to a first binding motif; and
b) a second nucleic acid construct comprising a polynucleotide sequence encoding a nucleic acid binding protein, or a fragment of the nucleic acid binding protein, fused to a second binding motif,
wherein the first binding motif and the second binding motif form a covalent bond via protein ligation when brought into contact with one another either spontaneously or with the help of an enzyme.

In some embodiments, a pair of nucleic acid constructs comprises:
a) a first nucleic acid construct comprising a polynucleotide sequence encoding a first non-retroviral retrotransposon, or a first fragment of the non-retroviral retrotransposon having reverse transcriptase activity, fused to a first binding motif; and
b) a second nucleic acid construct comprising a polynucleotide sequence encoding a second non-retroviral retrotransposon, or a second fragment of the non-retroviral retrotransposon having reverse transcriptase activity, fused to a second binding motif,
wherein the first binding motif and the second binding motif form a covalent bond via protein ligation when brought into contact with one another either spontaneously or with the help of an enzyme.

In an embodiment, a pair of nucleic acid constructs comprises:
a) a first nucleic acid construct comprising a polynucleotide sequence encoding a non-retroviral retrotransposon, or a fragment of the non-retroviral retrotransposon having reverse transcriptase activity, fused to a first acceptor peptide; and
b) a second nucleic acid construct comprising a polynucleotide sequence encoding a nucleic acid binding protein, or a fragment of the nucleic acid binding protein, fused to a second acceptor peptide,
wherein the first and second acceptor peptides are biotinylated in the presence of biotin, a biotin ligase, and adenosine triphosphate (ATP) to form biotinylated first and second acceptor peptides; and
wherein the biotinylated first and second acceptor peptides bind to a biotin-binding protein via non-covalent interactions when brought into contact with one another.

In some embodiments, a pair of nucleic acid constructs comprises:
a) a first nucleic acid construct comprising a polynucleotide sequence encoding a first non-retroviral retrotransposon, or a first fragment of the non-retroviral retrotransposon having reverse transcriptase activity, fused to a first acceptor peptide; and
b) a second nucleic acid construct comprising a polynucleotide sequence encoding a second non-retroviral retrotransposon, or a second fragment of the non-retroviral retrotransposon having reverse transcriptase activity, fused to a second acceptor peptide,
wherein the first and second acceptor peptides are biotinylated in the presence of biotin, a biotin ligase, and adenosine triphosphate (ATP) to form biotinylated first and second acceptor peptides; and
wherein the biotinylated first and second acceptor peptides bind to a biotin-binding protein via non-covalent interactions when brought into contact with one another.

The nucleic acid constructs are typically introduced into various vectors. The vectors described herein generally comprise transcriptional or translational control sequences required for expressing the fusion proteins. Suitable transcription or translational control sequences include, but are not limited to, replication origin, promoter, enhancer, repressor binding regions, transcription initiation sites, ribosome binding sites, translation initiation sites, and termination sites for transcription and translation.

The origin of replication (generally referred to as an on sequence) permits replication of the vector in a suitable host cell. The choice of on will depend on the type of host cells and/or genetic packages that are employed. Where the host cells are prokaryotes, the expression vector typically comprises ori sequences directing autonomous replication of the vector within the prokaryotic cells. Preferred prokaryotic on is capable of directing vector replication in bacterial cells. Non-limiting examples of this class of on include pMB1, pUC, as well as other E. coli origins.

As used herein, a “promoter” is a DNA region capable under certain conditions of binding RNA polymerase and initiating transcription of a coding region located downstream (in the 3′ direction) from the promoter. It can be constitutive or inducible. In general, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains responsible for the binding of RNA polymerase.

The choice of promoters will largely depend on the host cells in which the vector is introduced. For prokaryotic cells, a variety of robust promoters are known in the art. Preferred promoters are lac promoter, Trc promoter, T7 promoter and pBAD promoter.

In constructing the subject vectors, the termination sequences associated with the protein coding sequence can also be inserted into the 3′ end of the sequence desired to be transcribed to provide polyadenylation of the mRNA and/or transcriptional termination signal. The terminator sequence preferably contains one or more transcriptional termination sequences (such as polyadenylation sequences) and may also be lengthened by the inclusion of additional DNA sequence so as to further disrupt transcriptional read-through. Preferred terminator sequences (or termination sites) of the present invention have a gene that is followed by a transcription termination sequence, either its own termination sequence or a heterologous termination sequence. Examples of such termination sequences include stop codons coupled to various yeast transcriptional termination sequences or mammalian polyadenylation sequences that are known in the art and are widely available. Where the terminator comprises a gene, it can be advantageous to use a gene which encodes a detectable or selectable marker; thereby providing a means by which the presence and/or absence of the terminator sequence (and therefore the corresponding inactivation and/or activation of the transcription unit) can be detected and/or selected.

In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins, e.g., ampicillin, kanamycin, neomycin, zeocin, G418, methotrexate, etc.; (b) complement auxotrophic deficiencies; or (c) supply critical nutrients not available from complex media. The choice of the proper marker gene will depend on the host cell, and appropriate genes for different hosts are known in the art.

In one embodiment, the expression vector is a shuttle vector, capable of replicating in at least two unrelated host systems. In order to facilitate such replication, the vector generally contains at least two origins of replication, one effective in each host system. Typically, shuttle vectors are capable of replicating in a eukaryotic host system and a prokaryotic host system. This enables detection of protein expression in the eukaryotic host (the expression cell type) and amplification of the vector in the prokaryotic host (the amplification cell type). Preferably, one origin of replication is derived from SV40 or 2u and one is derived from pUC, although any suitable origin known in the art may be used provided it directs replication of the vector. Where the vector is a shuttle vector, the vector preferably contains at least two selectable markers, one for the expression cell type and one for the amplification cell type. Any selectable marker known in the art or those described herein may be used provided it functions in the expression system being utilized.

The vectors encompassed by the invention can be obtained using recombinant cloning methods (e.g., PCR, restriction endonuclease digestion and ligation methods) and/or by chemical synthesis. Sequence data provided herein or sequence data in the public or proprietary databases can be used to obtain a desired vector by any synthetic means available in the art. Additionally, by using restriction and ligation techniques, appropriate sequences can be excised from various DNA sources and integrated in operative relationship with the exogenous sequences to be expressed in accordance with embodiments.

### Methods of Producing Hybrid Reverse Transcriptases

The invention also provides methods for producing the disclosed hybrid reverse transcriptases. Methods of producing the hybrid reverse transcriptase include, but are not limited to, chemical methods, recombinant methods, and protein ligation methods in which polypeptides are covalently linked. Biotinylated acceptor peptides and a biotin-binding protein can also be used to join polypeptides via non-covalent interactions.

Chemical methods of joining polypeptides are described, for example, in Bioconjugate Techniques, Hermanson, Ed., Academic Press (1996). These chemical methods include, for example, derivitization for the purpose of linking the polypeptides to each other, either directly or through a linking compound, by methods that are well known in the art of protein chemistry. For example, a heterobifunctional-coupling reagent can be used to form an intermolecular disulfide bond between the polypeptides. Other types of coupling reagents that can be used to join polypeptides are described, for example, in U.S. Pat. No. 4,545,985. Alternatively, an intermolecular disulfide bond may be formed between cysteines in each polypeptide, which occur naturally or are inserted by genetic engineering. The means of linking the polypeptides may also use thioether linkages between heterobifunctional crosslinking reagents or specific low pH cleavable crosslinkers or specific protease cleavable linkers or other cleavable or noncleavable chemical linkages.

In some embodiments, nonclassical amino acids or chemical amino acid analogs can be introduced as a substitution or addition into one or all of the polypeptide sequences to be joined. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxy-proline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids,N-methyl amino acids, and amino acid analogs in general. Furthermore, the amino acid can be D (dextrorotary) or L (levorotary).

### Production of Hybrid Reverse Transcriptases Using Recombinant Techniques

In an embodiment, a hybrid reverse transcriptase is produced by recombinant expression of a nucleic acid encoding the protein. Such a protein can be made by fusing the appropriate nucleic acid sequences encoding the desired amino acid sequences to each other in the proper coding frame and then expressing the product in an appropriate expression system.

Nucleic acids encoding the hybrid reverse transcriptases can be obtained using routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-1999). Such nucleic acids may also be obtained through in vitro amplification methods such as those described in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Natl. Acad. Sci. USA 87, 1874; Lomeli et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

Modifications can additionally be made to the hybrid reverse transcriptases without diminishing their biological activity. Some modifications may be made to facilitate the cloning, expression, or incorporation of a domain into a fusion protein. Such modifications include, for example, the addition of codons at either terminus of the polynucleotide. For example, a codon that encodes an initiation site can be added to the amino terminus (e.g., a methionine added at the amino terminus). Additional amino acids (e.g., poly His) can also be placed on either terminus to create conveniently located restriction sites or termination codons or purification sequences.

Expression systems for producing the polypeptides are described by, for example, Gene Expression Systems, Fernandex and Hoeffler, Eds. Academic Press, 1999; Sambrook and Russell, supra; and Ausubel et al, supra. Typically, the polynucleotide that encodes the polypeptide is placed under the control of a promoter that is functional in the desired host cell. The promoter selected depends upon the cell in which the promoter is to be active. Other expression control sequences such as ribosome binding sites, transcription termination sites are also optionally included. Constructs that include one or more of these control sequences are termed “expression cassettes.” Accordingly, the nucleic acids that encode the joined polypeptides are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer, et al., Proc. Natl. Acad. Sci. U.S.A. (1983) 80:21-25); and the lambda-derived PL promoter and N-gene ribosome binding site (Shimatake et al., Nature (1981) 292: 128). The particular promoter system is not critical; any available promoter that functions in prokaryotes and provides the desired level of activity can be used.

Standard bacterial expression vectors include plasmids such as pBR322-based plasmids, e.g., pBLUESCRIPT™, pSKF, pET23D, lambda-phage derived vectors, and fusion expression systems such as GST and LacZ. The vector containing a nucleic acid encoding the hybrid reverse transcriptase can be transformed into a cell using standard techniques, for example, by employing chemical methods (Green R, Rogers E J. Transformation of chemically competent E. coli. Methods Enzymol 2013; 529:329-36) or by electroporation.

Polypeptides can be expressed in a variety of host cells, including E. coli, other bacterial hosts, yeast, and various higher eukaryotic cells such as the COS, CHO and HeLa cells lines and myeloma cell lines. The host cells can be mammalian cells, insect cells, or microorganisms, such as, for example, yeast cells, bacterial cells, or fungal cells. Cells able to express the one or more markers are able to survive/grow/multiply under certain artificially imposed conditions, for example the addition of a toxin or antibiotic to the culture medium, because of the properties endowed by the polypeptide/gene or polypeptide component of the selection system incorporated therein (e.g., antibiotic resistance). Those cells that cannot express the one or more markers are not able to survive/grow/multiply in the artificially imposed conditions.

Any suitable selection system may be employed in the expression methods. Typically the selection system may be based on including in the vector one or more genes that provides resistance to a known antibiotic, for example a tetracycline, chloramphenicol, kanamycin or ampicillin resistance gene. Cells that grow in the presence of a relevant antibiotic can be selected as they express both the gene that gives resistance to the antibiotic and the desired protein.

In an embodiment, the method further comprises the step of culturing the transformed cell in a medium to thereby express the hybrid reverse transcriptase. Any suitable medium may be used to culture the transformed cell. The medium may be adapted for a specific selection system, for example the medium may comprise an antibiotic, to allow only those cells which have been successfully transformed to grow in the medium.

The hybrid reverse transcriptases can be expressed intracellularly, or can be secreted from the cell. Intracellular expression often results in high yields. If necessary, the amount of soluble, active fusion polypeptide may be increased by performing refolding procedures (see, e.g., Sambrook et al., supra.; Marston et al., Bio/Technology (1984) 2: 800; Schoner et al., Bio/Technology (1985) 3: 151).

Once expressed, the hybrid reverse transcriptases can be purified according to standard procedures of the art, including ammonium sulfate precipitation, affinity columns, column chromatography, gel electrophoresis, dialysis, ammonium sulphate, ethanol or PEG fractionation/precipitation, ion exchange membranes, expanded bed adsorption chromatography, or simulated moving bed chromatography. Substantially pure compositions of at least about 90 to 95% homogeneity are preferred, and 98 to 99% or more homogeneity are most preferred. Once purified, partially or to homogeneity as desired, the polypeptides may then be used (e.g., in disclosed methods to generate a cDNA molecule or cDNA library).

To facilitate purification of the polypeptides, the nucleic acids that encode the polypeptides can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1N5-His are suitable for expression in mammalian cells). Additional expression vectors suitable for attaching a tag to the fusion proteins of the invention, and corresponding detection systems are commercially available (e.g., “FLAG” (Kodak, Rochester N.Y.)). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” in Genetic Engineering: Principles and Methods, J. K. Setlow, Ed., Plenum Press, N.Y.; commercially available from Qiagen (Santa Clarita, Calif.)).

After biological expression or purification, the hybrid reverse transcriptases may possess a conformation substantially different than the native conformations of the constituent polypeptides. In this case, it may be necessary or desirable to denature and reduce the polypeptide and then to cause the polypeptide to re-fold into the preferred conformation. A method of reducing and denaturing proteins and inducing re-folding is described, for example, in Debinski et al. (1993) J. Biol. Chem. 268: 14065-14070.

In some embodiments, the method further comprises measuring the quantity of expression of the hybrid reverse transcriptase after purification.

### Production of Hybrid Reverse Transcriptases Using Protein Ligation

Also provided are methods for producing the disclosed hybrid reverse transcriptases via protein ligation. To produce a hybrid reverse transcriptase, a binding motif is first recombinantly fused to the non-retroviral retrotransposon, or the fragment of the non-retroviral retrotransposon having reverse transcriptase activity, and the nucleic acid binding protein or the fragment of the nucleic acid binding protein. Any of the protein ligation systems described previously or otherwise known in the art can be used to design binding motifs that are fused to the non-retroviral retrotransposon, or the fragment of the non-retroviral retrotransposon, and the nucleic acid binding protein, or the fragment of the nucleic acid binding protein, to produce a hybrid reverse transcriptase.

In some embodiments, a first fusion protein is produced with a first binding motif N- or C-terminally fused to a non-retroviral retrotransposon, or a fragment of the non-retroviral retrotransposon having reverse transcriptase activity. A second fusion protein is also produced with a second binding motif N- or C-terminally fused to a nucleic acid binding protein or a fragment of the nucleic acid binding protein, depending on which ends of the fusion proteins are to be ligated.

In a dimeric embodiment of the hybrid reverse transcriptase, a first fusion protein is produced with a first binding motif N- or C-terminally fused to a first non-retroviral retrotransposon, or a first fragment of the non-retroviral retrotransposon having reverse transcriptase activity. A second fusion protein is also produced with a second binding motif N- or C-terminally fused to a second non-retroviral retrotransposon, or a second fragment of the non-retroviral retrotransposon having reverse transcriptase activity, depending on which ends of the fusion proteins are to be ligated.

Interchangeable binding motif cognate pairs are used in the first and second fusion proteins. For example, if the first binding motif SpyTag or SpyTag002 is fused to the C-terminus of the non-retroviral retrotransposon, or the fragment of the non-retroviral retrotransposon, then the second binding motif SpyCatcher, SpyCatcher short or SpyCatcher002 is fused to the N-terminus of the nucleic acid binding protein or the fragment of the nucleic acid binding protein. Alternatively, if SpyCatcher, SpyCatcher short or SpyCatcher002 is fused to the C-terminus of the non-retroviral retrotransposon, or the fragment of the non-retroviral retrotransposon, then SpyTag or SpyTag002 is fused to the N-terminus of the nucleic acid binding protein or the fragment of the nucleic acid binding protein. Other combinations of binding motif pairs include, but are not limited to: SnoopTag and SnoopCatcher, Isopeptag and Split Spy0128, SdyTag and SdyCatcherDANG short, SpyTag and K-Tag, SnoopTagJr and DogTag, and sortase recognition domain and sortase bridging domain (see Table 6).

In an embodiment, the first and second fusion proteins are produced by recombinant expression of nucleic acids encoding the fusion proteins. Such proteins can be made by fusing the appropriate nucleic acid sequences encoding the desired amino acid sequences to each other in the proper coding frame and then expressing the product in an appropriate expression system and by processes described previously. The expressed fusion proteins can then be purified by methods described previously.

### Ligation

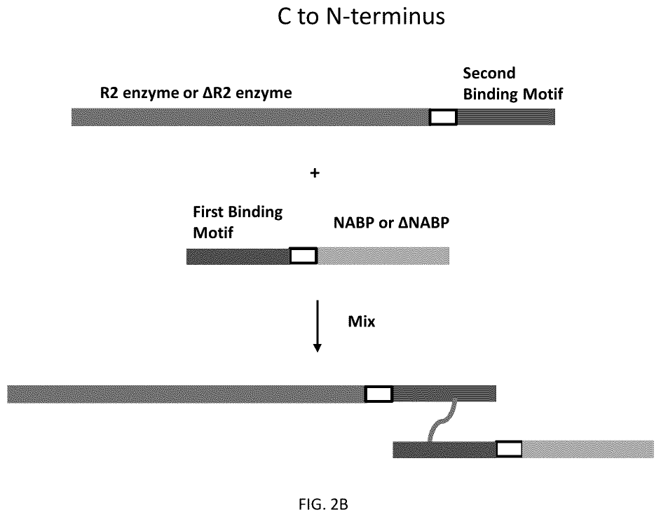

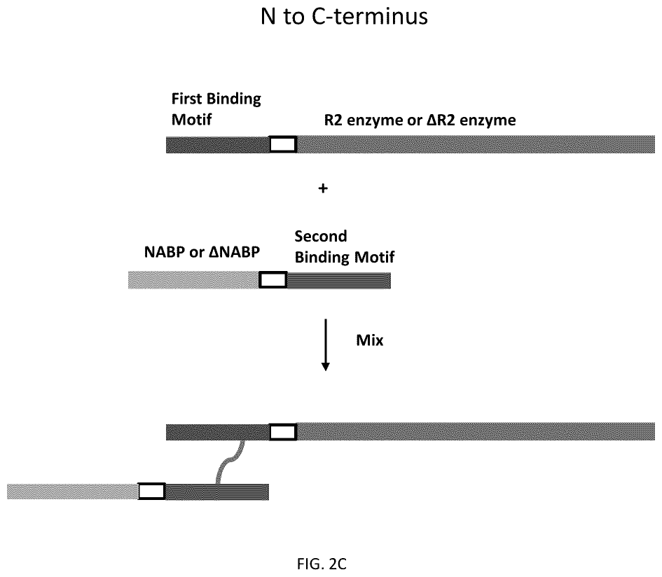

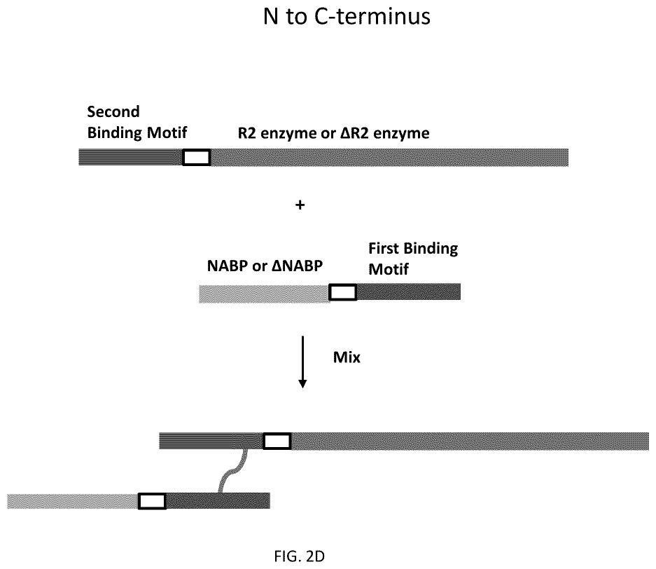

To produce the hybrid reverse transcriptase, the first and second fusion proteins are mixed with each other under appropriate conditions to allow the first and second binding motifs in the first and second fusion proteins, respectively, to covalently join via protein ligation, either spontaneously or with the help of an enzyme. In an embodiment, the hybrid reverse transcriptase comprises a first fusion protein having a C-terminal or N-terminal binding motif conjugated to a C-terminal or N-terminal binding motif of the second fusion protein in any combination, i.e., the first and second fusion proteins can be attached via C-terminus to N-terminus, N-terminus to C-terminus, N-terminus to N-terminus, or C-terminus to C-terminus via the binding motifs (FIGS. 2A-5C). For example, in an embodiment using an R2 enzyme lacking an N-terminal DNA binding domain (i.e., ΔR2 enzyme) and a SpyTag/SpyCatcher binding motif pair, a ΔR2 enzyme-SpyTag fusion protein is mixed with a SpyCatcher-nucleic acid binding protein fusion to produce a hybrid reverse transcriptase having the ΔR2 enzyme with the C-terminal SpyTag conjugated to the N-terminal SpyCatcher of the nucleic acid binding protein (FIG. 2A). Cognate binding motif pairs are interchangeable; thus, in some embodiments, a ΔR2 enzyme-SpyCatcher fusion protein is mixed with a SpyTag-nucleic acid binding protein fusion to produce a hybrid reverse transcriptase having the ΔR2 enzyme with the C-terminal SpyCatcher conjugated to the N-terminal SpyTag of the nucleic acid binding protein (FIG. 2B). Alternatively, a SpyTag-ΔR2 enzyme fusion protein can be mixed with a nucleic acid binding protein-SpyCatcher fusion protein to produce a hybrid reverse transcriptase having the ΔR2 enzyme with the N-terminal SpyTag conjugated to the C-terminal SpyCatcher of the nucleic acid binding protein (FIG. 2C). Also, a SpyCatcher-ΔR2 enzyme fusion protein can be mixed with a nucleic acid binding protein-SpyTag fusion protein to produce a hybrid reverse transcriptase having the ΔR2 enzyme with the N-terminal SpyCatcher conjugated to the C-terminal SpyTag of the nucleic acid binding protein (FIG. 2D).

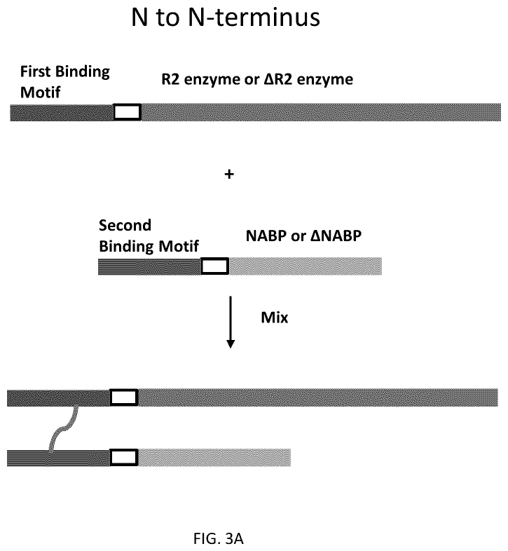

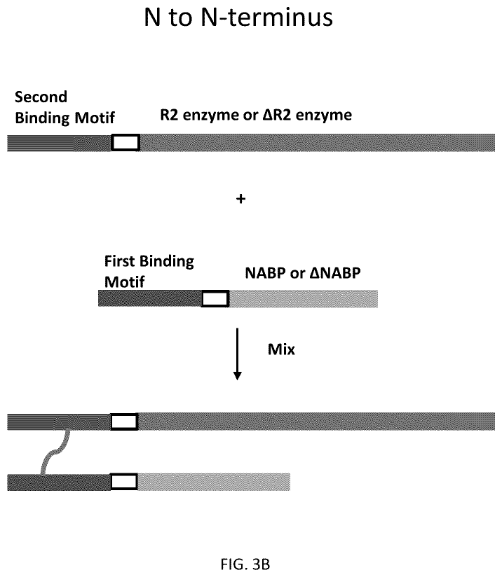

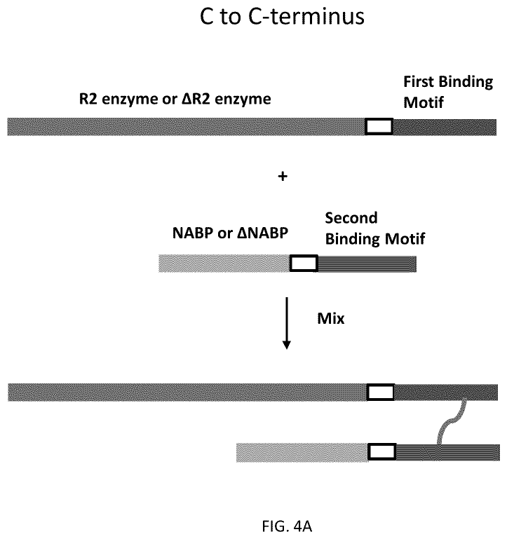

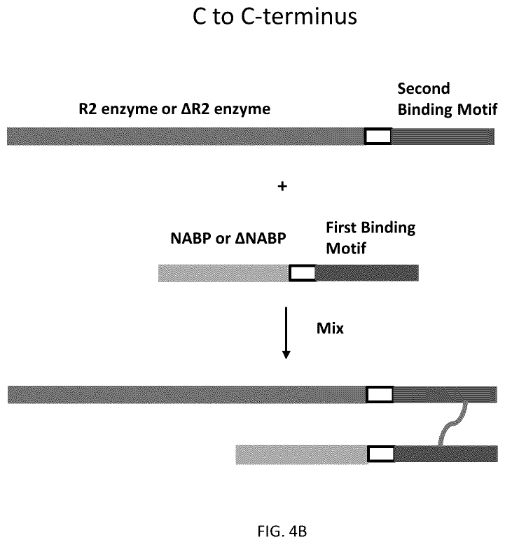

In some embodiments, a SpyTag-ΔR2 enzyme fusion protein is mixed with a SpyCatcher-nucleic acid binding protein fusion protein to produce a hybrid reverse transcriptase having the ΔR2 enzyme with the N-terminal SpyTag conjugated to the N-terminal SpyCatcher of the nucleic acid binding protein (FIG. 3A). Likewise, a SpyCatcher-ΔR2 enzyme fusion protein can be mixed with a SpyTag-nucleic acid binding protein fusion protein to produce a hybrid reverse transcriptase having the ΔR2 enzyme with the N-terminal SpyCatcher conjugated to the N-terminal SpyTag of the nucleic acid binding protein (FIG. 3B). In certain embodiments, a ΔR2 enzyme-SpyTag fusion protein is mixed with a nucleic acid binding protein-SpyCatcher fusion protein to produce a hybrid reverse transcriptase having the ΔR2 enzyme with the C-terminal SpyTag conjugated to the C-terminal SpyCatcher of the nucleic acid binding protein (FIG. 4A). Likewise, a ΔR2 enzyme-SpyCatcher fusion protein can be mixed with a nucleic acid binding protein-SpyTag fusion protein to produce a hybrid reverse transcriptase having the ΔR2 enzyme with the C-terminal SpyCatcher conjugated to the C-terminal SpyTag of the nucleic acid binding protein-SpyTag fusion protein (FIG. 4B).

In some embodiments, the produced hybrid reverse transcriptase comprises a first non-retroviral retrotransposon, or a first fragment of the non-retroviral retrotransposon, conjugated to a second non-retroviral retrotransposon, or a second fragment of the non-retroviral retrotransposon (i.e., a dimer) via C-terminus to N-terminus N-terminus to N-terminus, or C-terminus to C-terminus by protein ligation (FIGS. 5A-5C). For example, in an embodiment, a first ΔR2 enzyme-SpyTag fusion protein is mixed with a SpyCatcher-second ΔR2 enzyme fusion protein to produce a hybrid reverse transcriptase having the first ΔR2 enzyme with the C-terminal SpyTag conjugated to the N-terminal SpyCatcher of the second ΔR2 enzyme (FIG. 5A). The SpyTag and SpyCatcher binding motifs are interchangeable; thus, in some embodiments, a first ΔR2 enzyme-SpyCatcher fusion protein is mixed with a SpyTag-second ΔR2 enzyme fusion protein to produce a hybrid reverse transcriptase having the first ΔR2 enzyme with the C-terminal SpyCatcher conjugated to the N-terminal SpyTag of the second ΔR2 enzyme. In some embodiments, a SpyTag-first ΔR2 enzyme fusion protein is mixed with a SpyCatcher-second ΔR2 enzyme fusion protein to produce a hybrid reverse transcriptase having the first ΔR2 enzyme with the N-terminal SpyTag conjugated to the N-terminal SpyCatcher of the nucleic acid binding protein (FIG. 5B). In certain embodiments, a first ΔR2 enzyme-SpyTag fusion protein is mixed with a second ΔR2 enzyme-SpyCatcher fusion protein to produce a hybrid reverse transcriptase having the first ΔR2 enzyme with the C-terminal SpyTag conjugated to the C-terminal SpyCatcher of the second ΔR2 enzyme (FIG. 5C).

In some embodiments, a SnoopTag/SnoopCatcher protein ligation system (Veggiani et al., 2016) is used to produce a hybrid reverse transcriptase. In such embodiments, a non-retroviral retrotransposon, or a fragment of the non-retroviral retrotransposon having reverse transcriptase activity fused to SnoopTag or SnoopCatcher and a nucleic acid binding protein, or a fragment of the nucleic acid binding protein, fused to SnoopCatcher or SnoopTag, respectively, is produced as described previously. The fusion proteins are then mixed under appropriate conditions to allow ligation of SnoopTag to SnoopCatcher.

In certain embodiments, an Isopeptag/Split Spy0128 protein ligation system (Abe et al., 2013) is used to produce a hybrid reverse transcriptase. In such embodiments, a non-retroviral retrotransposon, or a fragment of the non-retroviral retrotransposon having reverse transcriptase activity fused to Isopeptag or Split Spy0128 and a nucleic acid binding protein fused to Split Spy0128 or Isopeptag, respectively, is produced as described previously. The fusion proteins are then mixed under appropriate conditions to allow ligation of Isopeptag to Split Spy0128.

In some embodiments, a SdyTag/SdyCatcherDANG short protein ligation system (Tan et al., 2016) is used to produce a hybrid reverse transcriptase. In such embodiments, a non-retroviral retrotransposon, or a fragment of the non-retroviral retrotransposon having reverse transcriptase activity fused to SdyTag or SdyCatcherDANG short and a nucleic acid binding protein, or a fragment of the nucleic acid binding protein, fused to SdyCatcherDANG short or SdyTag, respectively, is produced as described previously. The fusion proteins are then mixed under appropriate conditions to allow ligation of SdyTag to SdyCatcherDANG short.

In certain embodiments, the SpyLigase or SnoopLigase protein ligation systems can be used to create a hybrid reverse transcriptase. For example, if the SpyLigase protein ligation system is used, a non-retroviral retrotransposon, or a fragment of the non-retroviral retrotransposon having reverse transcriptase activity fused to SpyTag can be produced as described previously. The second binding motif (SpyCatcher) in the nucleic acid binding protein, or a fragment of the nucleic acid binding protein, is replaced by the 10 amino acid K-Tag binding motif (SEQ ID NO: 55). The fusion proteins are then mixed in the presence of SpyLigase under conditions that permit the ligation of SpyTag and K-tag. Alternatively, if the SnoopLigase protein ligation system is used, the SnoopTag binding motif is replaced with SnoopTagJr and the SnoopCatcher binding motif is replaced with the 23 amino acid DogTag binding motif (SEQ ID NO: 61). First and second fusion proteins having SnoopTagJr and DogTag binding motifs, respectively, are mixed in the presence of SnoopLigase under conditions that permit ligation of SnoopTagJr and DogTag. For example, a non-retroviral retrotransposon, or a fragment of the non-retroviral retrotransposon having reverse transcriptase activity fused to SnoopTagJr can be mixed with a nucleic acid binding protein fused to DogTag in the presence of SnoopLigase under conditions that allow ligation of SnoopTagJr and DogTag to produce a hybrid reverse transcriptase.

In further embodiments, the Sortase system is used to produce a hybrid reverse transcriptase. For example, a non-retroviral retrotransposon, or a fragment of the non-retroviral retrotransposon having reverse transcriptase activity fused to sortase recognition domain (i.e., any of SEQ ID NOs: 62-68) and a nucleic acid binding protein fused to a sortase bridging domain (i.e., any of Gly, (Gly)₂, (Gly)₃, (Gly)₄, or (Gly)ₓ, where x is an integer of 1-20) can be produced as described above. The fusion proteins are then mixed in the presence of sortase to allow covalent coupling of the sortase recognition and bridging domains.

### Production of Hybrid Reverse Transcriptases Using Biotinylated Acceptor Peptides and Biotin-Binding Protein

The invention also provides methods for producing the disclosed hybrid reverse transcriptases. To produce a hybrid reverse transcriptase, an acceptor peptide is first recombinantly fused to the non-retroviral retrotransposon, or the fragment of the non-retroviral retrotransposon having reverse transcriptase activity, and to the nucleic acid binding protein, or a fragment of the nucleic acid binding protein.

In some embodiments, a first fusion protein is produced with a first acceptor peptide N- or C-terminally fused to a non-retroviral retrotransposon, or a fragment of the non-retroviral retrotransposon having reverse transcriptase activity. A second fusion protein is also produced with a second acceptor peptide N- or C-terminally fused to a nucleic acid binding protein, or a fragment of the nucleic acid binding protein, depending on which ends of the fusion proteins are to be attached to a biotin-binding protein.

In certain embodiments of the hybrid reverse transcriptase, a first fusion protein is produced with a first acceptor peptide N- or C-terminally fused to a first non-retroviral retrotransposon, or a first fragment of the non-retroviral retrotransposon having reverse transcriptase activity. A second fusion protein is also produced with a second acceptor peptide N- or C-terminally fused to a second non-retroviral retrotransposon, or a second fragment of the non-retroviral retrotransposon having reverse transcriptase activity, depending on which ends of the fusion proteins are to be attached to a biotin-binding protein.

The first and second acceptor peptides can be identical or different and are any of SEQ ID NOS: 85-89.

In an embodiment, the first and second fusion proteins are produced by recombinant expression of nucleic acids encoding the fusion proteins. Such proteins are made by fusing the appropriate nucleic acid sequences encoding the desired amino acid sequences to each other in the proper coding frame and then expressing the product in an appropriate expression system and by processes described previously. The expressed fusion proteins can then be purified by methods described previously.

In some embodiments, the first and second acceptor peptides are biotinylated in vitro by contacting the fusion proteins with biotin in the presence of biotin ligase and ATP to form biotinylated first and second acceptor peptides.

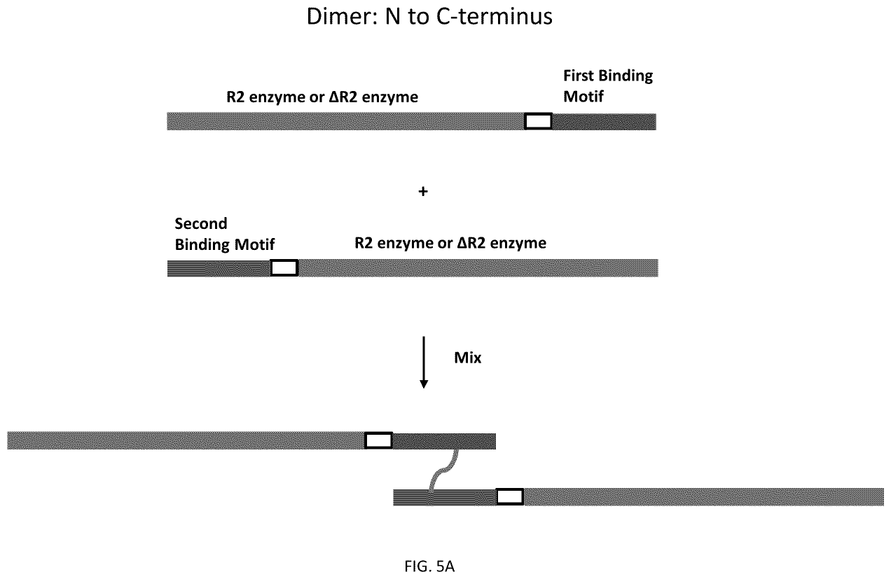

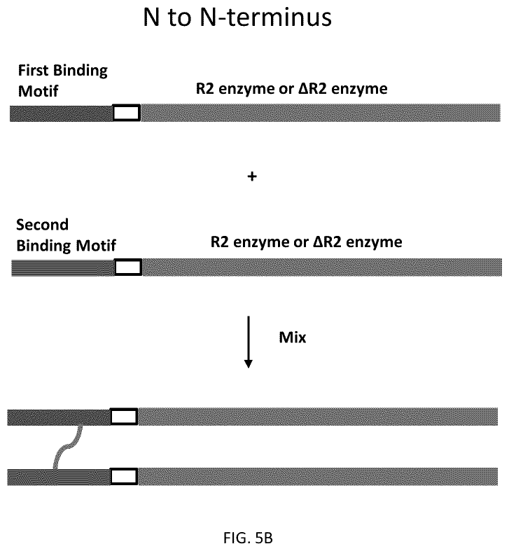

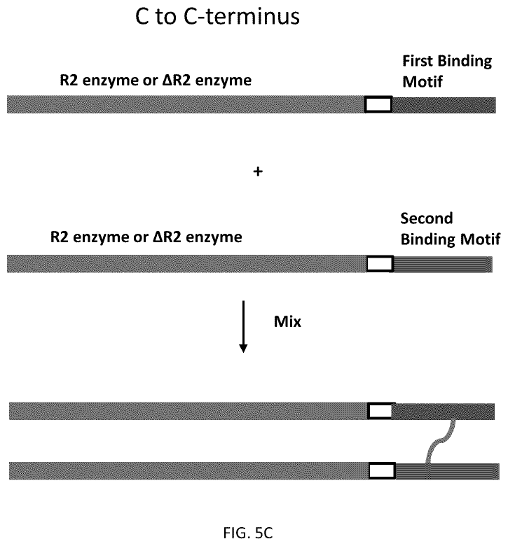

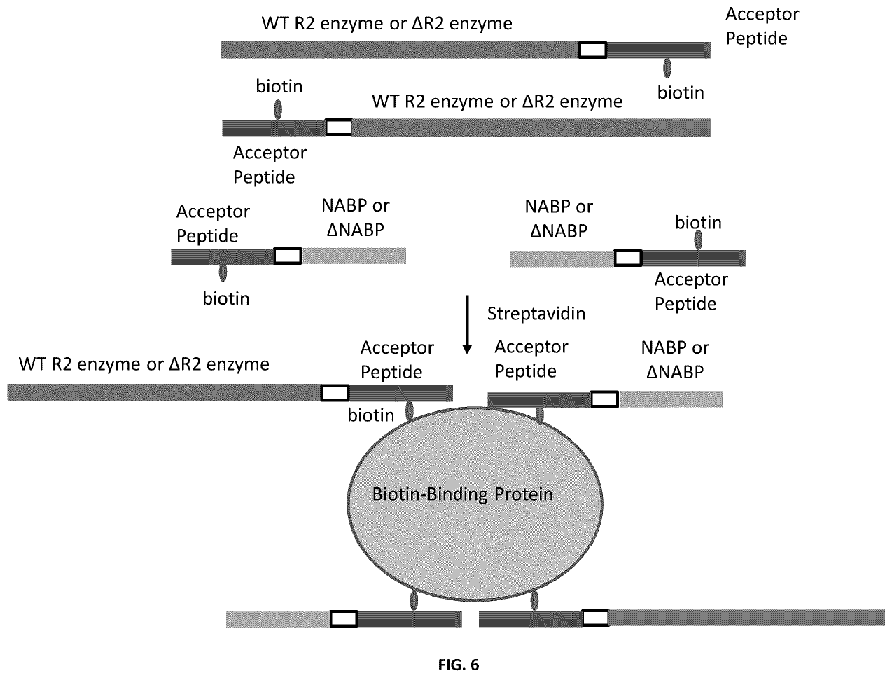

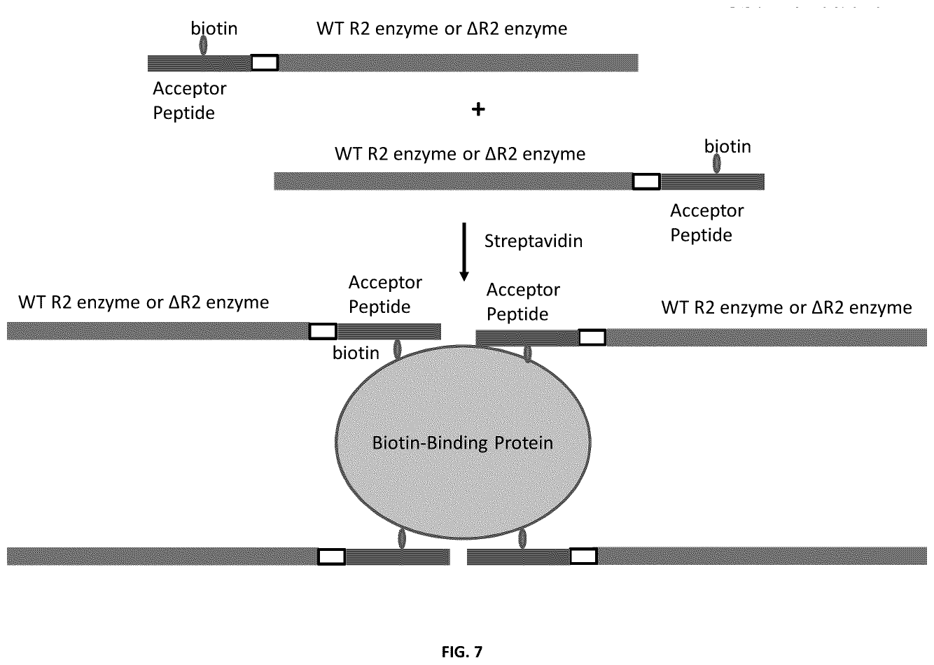

To produce the hybrid reverse transcriptase, the biotinylated first and second fusion proteins are mixed with a biotin-binding protein under appropriate conditions to allow the first and second acceptor peptides in the respective first and second fusion proteins to non-covalently bind to the biotin-binding protein. In an embodiment, the hybrid reverse transcriptase comprises a first fusion protein having a C-terminal or N-terminal biotinylated acceptor peptide and a second fusion protein having a C-terminal or N-terminal biotinylated acceptor peptide non-covalently bound to the biotin-binding protein via biotin (FIGS. 6-7). For example, in an embodiment using an R2 enzyme lacking an N-terminal DNA binding domain (i.e., ΔR2 enzyme), a ΔR2 enzyme-biotinylated acceptor peptide fusion protein, a biotinylated acceptor peptide-ΔR2 enzyme fusion protein, a biotinylated acceptor peptide-nucleic acid binding protein (NABP) fusion, a nucleic acid binding protein-biotinylated acceptor peptide fusion protein, and a biotin-binding protein are mixed together to produce a hybrid reverse transcriptase having ΔR2 enzymes with C- and N-terminal biotinylated acceptor peptides and nucleic acid binding proteins with C- and N-terminal biotinylated acceptor peptides bound to biotin-binding protein (FIG. 6). In an embodiment using wild type R2 enzyme (i.e., WT R2 enzyme; SEQ ID NO: 1), an WT R2 enzyme-biotinylated acceptor peptide fusion protein, a biotinylated acceptor peptide-WT R2 enzyme fusion protein, a biotinylated acceptor peptide-nucleic acid binding protein fusion, a nucleic acid binding protein-biotinylated acceptor peptide fusion protein, and a biotin-binding protein are mixed together to produce a hybrid reverse transcriptase having the WT R2 enzymes with C- and N-terminal biotinylated acceptor peptides and nucleic acid binding proteins with C- and N-terminal biotinylated acceptor peptides bound to biotin-binding protein. In some embodiments, a fragment of the nucleic acid binding protein (i.e., ΔNABP) N- or C-terminally fused to biotinylated acceptor peptide is used in place of the nucleic acid binding protein N- or C-terminally fused to biotinylated acceptor peptide (FIG. 6).

In some embodiments, the produced hybrid reverse transcriptase comprises a first non-retroviral retrotransposon, or a first fragment of the non-retroviral retrotransposon, comprising a biotinylated first acceptor peptide, and a second non-retroviral retrotransposon, or a second fragment of the non-retroviral retrotransposon, comprising a biotinylated second acceptor peptide are non-covalently bound to a biotin-binding protein (FIG. 7). For example, in an embodiment, a first ΔR2 enzyme-biotinylated first acceptor peptide fusion protein, a biotinylated second acceptor peptide-second ΔR2 enzyme fusion protein, and a biotin-binding protein are mixed together to produce a hybrid reverse transcriptase in which the biotinylated acceptor peptides from the fusion proteins are non-covalently bound to the biotin-binding protein. In another embodiment, a first WT R2 enzyme-biotinylated-first acceptor peptide fusion protein, a biotinylated second acceptor peptide-second WT R2 enzyme fusion protein, and a biotin-binding protein are mixed together to produce a hybrid reverse transcriptase in which the biotinylated acceptor peptides from the fusion proteins are non-covalently bound to the biotin-binding protein.

### Methods of Using Hybrid Reverse Transcriptases

Also provided are methods of using the disclosed hybrid reverse transcriptases to prepare a cDNA molecule or a cDNA library from total-RNA, messenger RNA (mRNA), micro RNA (miRNA), ribosomal RNAs, transfer RNAs (tRNAs), long non-coding RNA, cell free-RNA or from a single cell. RNA used in the disclosed methods can be synthetic or derived from naturally occurring sources. In one embodiment, RNA is isolated from a biological sample containing a variety of other components, such as proteins, lipids and non-template nucleic acids. RNA can be obtained from any cellular material from an animal, plant, bacterium, fungus, or any other cellular organism. RNA can be obtained directly from an organism or from a biological sample obtained from an organism, e.g., from blood, urine, cerebrospinal fluid, seminal fluid, saliva, sputum, stool and tissue. Any tissue or body fluid specimen may be used as a source of RNA. RNA can also be isolated from cultured cells, such as a primary cell culture or a cell line. The cells or tissues from which RNA is obtained can be infected with a virus or other intracellular pathogen. In addition, RNA can be obtained from non-cellular or non-tissue samples, such as viral samples, or environmental samples. Generally, RNA can be extracted from a biological sample by a variety of techniques such as those described by Sambrook and Russell, Molecular Cloning: A Laboratory Manual, Third Edition, Cold Spring Harbor, N.Y. (2001).

In methods of using the disclosed hybrid reverse transcriptases to prepare a cDNA molecule or a cDNA library, reverse transcription is initiated from a primer that optionally hybridizes or partially hybridizes with a template RNA molecule (i.e., the primer may or may not be complementary to the template RNA molecule). In methods in which the primer hybridizes or partially hybridizes with the template RNA molecule, the hybrid reverse transcriptase comprises a dsDNA binding protein. In methods in which the primer does not hybridize with the template RNA molecule (i.e., the primer is not complementary to the template RNA molecule), the hybrid reverse transcriptase comprises a ssDNA binding protein. In the methods, after transcribing the template RNA molecule, the hybrid reverse transcriptase jumps from a 5′ end of the template RNA molecule to a 3′-end of an acceptor-adapter and continues reverse transcribing the acceptor-adaptor. Such template jumping produces full length reverse transcripts of the template RNA molecule. In some embodiments, the jumping of the hybrid reverse transcriptase to the 3′-end of the acceptor-adapter is independent of sequence identity between the template RNA molecule(s) and the acceptor-adapter.

Methods in which Hybrid Reverse Transcriptase Comprises a dsDNA Binding Protein

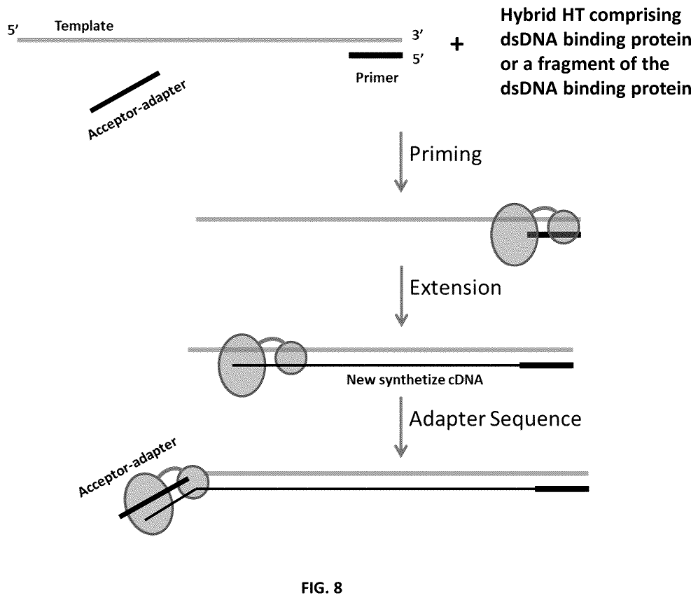

In an embodiment, a method of preparing a cDNA molecule comprises contacting a template RNA molecule and free nucleotides, with a primer that is complementary to the template RNA molecule, an acceptor-adapter, and a hybrid reverse transcriptase comprising a dsDNA binding protein or a fragment of a dsDNA binding protein (FIG. 8). The hybrid reverse transcriptase is then allowed to transcribe the template RNA molecule under conditions effective to produce a cDNA molecule complementary to the template RNA molecule and, optionally, to the acceptor-adapter. In some embodiments, the template RNA molecule comprises a poly (A) tail.

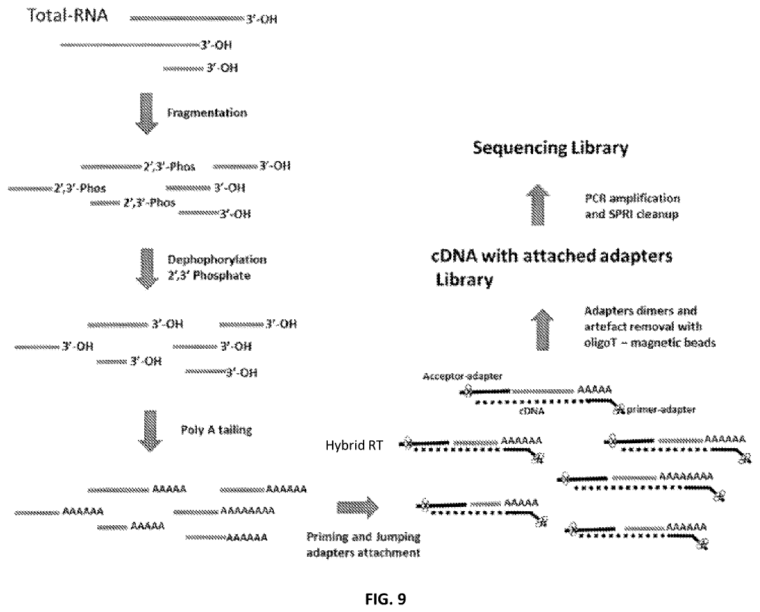

Referring to FIG. 9, in some embodiments, step (a) of a method for preparing a cDNA molecule library comprises fragmenting a template RNA molecule (e.g., total RNA) to produce RNA fragments. The template RNA can be fragmented by enzymatic or non-enzymatic methods. Enzymatic methods can use endonucleases with RNase activity (e.g., RNase A, RNase P, RNase H, RNase III, RNase T1, RNase T2, RNase U2, RNase VI, RNase I, RNase L, RNase PhyM, RNase V, dicer, or argonaute). Non-enzymatic methods take advantage of the natural chemical instability of RNAs. RNA can undergo spontaneous non-enzymatic fragmentation as a result of internal transphosphorylation. Breaking of phosphodiester bonds of RNA can be brought about by various conditions such as high pH or high temperature (e.g., about 70° C.-100° C.). RNA can also be fragmented in the presence of metals (e.g., Mg, Mn, Pb), polyamines, or cofactors, such as polyvinylpyrrolidone or polyethylene glycol. Non-enzymatic methods of fragmenting template RNA are more simple, are independent of enzyme activity or shelf life, and can be conducted under conditions compatible with the majority of the subsequent steps in the method.

The RNA fragments from the step (a) are a mixture of RNA fragments having a 3′-OH, a 2′,3′-cyclic phosphate, or a 3′-phosphate. The 2′,3′-cyclic phosphate can occur naturally (i.e., via cell free RNA degradation) or as a result of sample treatment or storage. RNA samples bearing 2′,3′-cyclic phosphate or 3′-phosphate cannot be subsequently poly-tailed or ligated because the presence of a free 3′-OH is required for both. Accordingly, in step (b) of the method, the 2′,3′-cyclic phosphate and the 3′-phosphate is removed from the RNA fragments by treating the RNA fragments with a phosphatase to generate dephosphorylated RNA fragments that can have a poly (A) tail added to the 3′-OH in step (c) of the method. In some embodiments, the phosphatase is T4 polynucleotide kinase (PNK). Other exemplary phosphatases include, but are not limited to, T4 polynucleotide kinase/phosphatase (Pnkp), Clostridium thermocellum Pnkp, yeast and plant tRNA ligase, and E. coli RtcB (Das and Shuman, “Mechanism of RNA 2′,3′-cyclic phosphate end healing by T4 polynucleotide kinase phosphatase,” Nucleic Acids Research, 2013, Vol. 41, No. 1, pp. 355-365).

In step (c) of the method, a poly (A) tail is added to the 3′-hydroxyl of the dephosphorylated RNA fragments by treating the dephosphorylated RNA fragments with, for example, poly-A polymerase.

A primer-adapter comprising an oligo-T sequence, an acceptor-adapter, nucleotides, and a hybrid reverse transcriptase comprising a dsDNA binding protein, or a fragment of the dsDNA binding protein, are then added to the poly-A tailed RNA fragments in step (d) of the method.

In step (e) of the method, the hybrid reverse transcriptase is allowed to transcribe the poly-A tailed RNA fragments under conditions effective to produce a cDNA molecule library. The hybrid reverse transcriptase then jumps to a 3′-end of the acceptor-adapter upon reaching the 5′ end of the poly-A tailed RNA fragments.

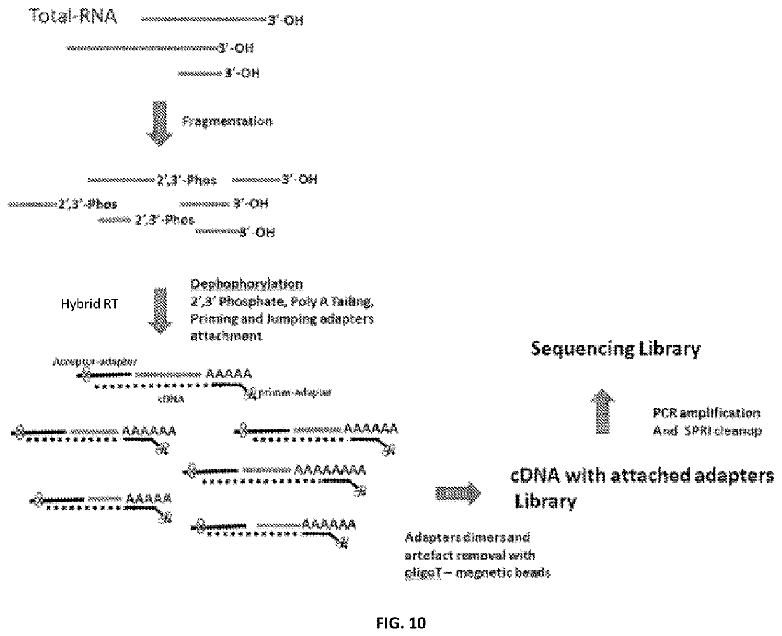

In some embodiments (FIG. 10), steps (b) and (c) are combined into one step by using a mutant form of T4 PNK in which the kinase enzymatic activity is removed. Kinase enzymatic activity can be removed by, for example, modifying one or more amino acid in the kinase domain of the enzyme (Wang and Shuman, “Domain Structure and Mutational Analysis of T4 Polynucleotide Kinase,” J. Biol. Chem. 2001, vol. 279, No. 29, pp 26868-26874).

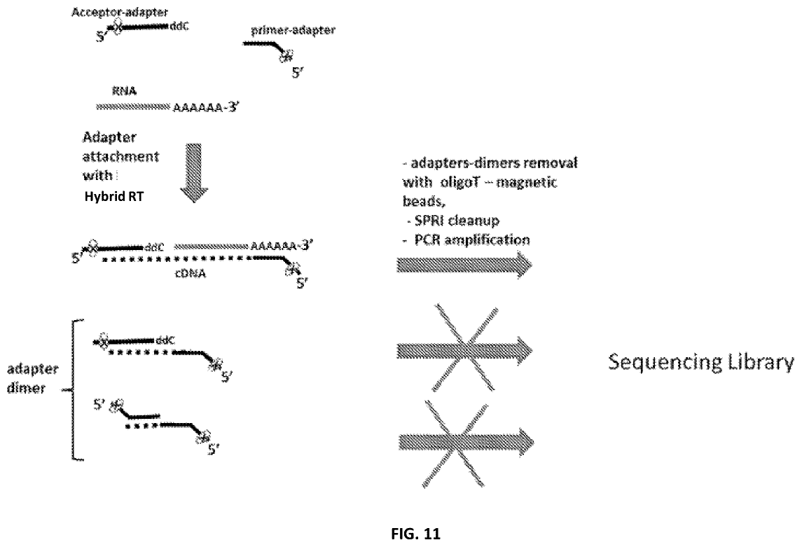

During the transcription (i.e., step (e)), non-annealed primer-adapter artifacts can be generated. Such artifacts include primer-adapter-acceptor-adapter dimers or homogenous primer-adapter dimers. Generation of primer-adapter-acceptor-adapter dimers can be prevented by including a 3′-dideoxy nucleotide at the 3′ end of the acceptor-adapter, which prevents acceptor-adapter extension. In some embodiments, one or more non-annealed primer-adapters are removed after the last step with an immobilized poly A oligonucleotide (e.g., oligo A attached to magnetic beads; FIG. 11) that hybridizes to an poly T sequence at the 3′end of the primer-adapter. Solid phase reverse immobilization (SPRI) can also be used in place of or after the immobilized poly A oligonucleotide are used to remove the non-annealed primer-adapter artifacts.

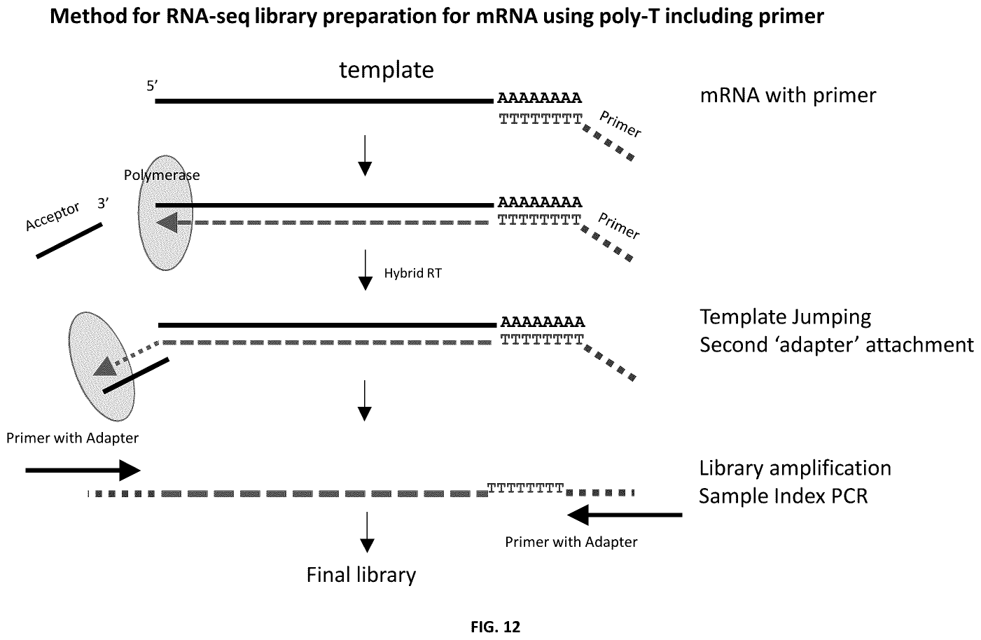

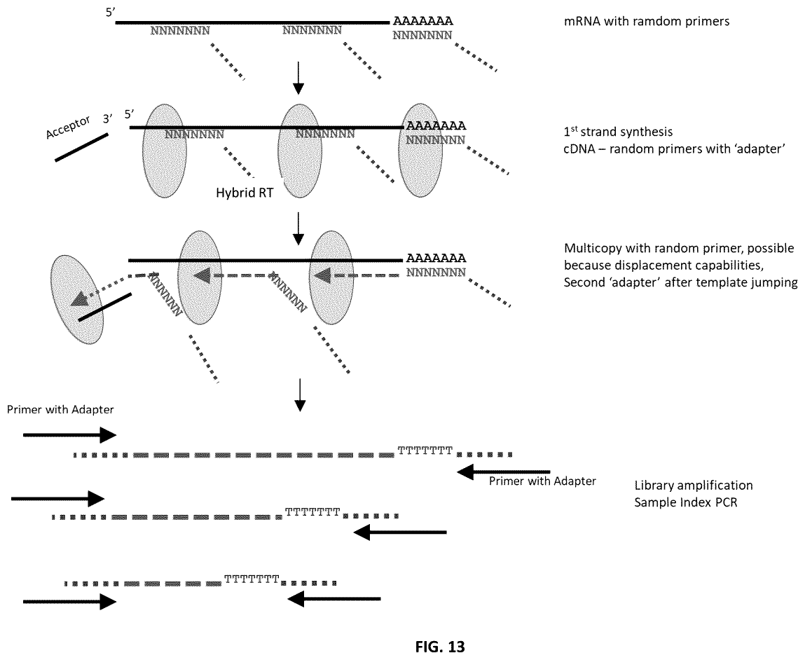

The disclosed hybrid reverse transcriptases can also be used to prepare cDNA from an mRNA template with a single primer-adapter (FIG. 12) or with random primer-adapters (FIG. 13). Referring to FIG. 12, in certain embodiments, step (a) of a method of preparing a cDNA molecule library using a single primer-adapter comprises annealing a primer-adapter comprising a poly (T) tail to a template RNA molecule comprising a poly (A) tail (e.g., a messenger RNA molecule comprising a poly (A) tail), thereby generating an annealed RNA molecule. In step (b) of the method, the annealed RNA molecule and free nucleotides are mixed, with an acceptor-adapter and a hybrid reverse transcriptase comprising a dsDNA binding protein, or a fragment of the dsDNA binding protein. In the next step (i.e., step (c)) of the method, the hybrid reverse transcriptase is allowed to transcribe the annealed RNA molecule at a temperature from about 12° C. to about 42° C. to produce a cDNA molecule library. In some embodiments, in step (c), the hybrid reverse transcriptase transcribes the annealed RNA molecule at a temperature of about 4° C. to about 50° C. (e.g., about 4° C., about 15° C., about 20° C., about 25° C., about 30° C., about 35° C., about 40° C., about 45° C., about 48° C.). In some embodiments, steps (a)-(c) are performed in a single reaction vessel. In certain embodiments, step (a) or step (b) further comprises adding hot start thermostable polymerase.

Referring to FIG. 13, in some embodiments, step (a) of a method for preparing a cDNA molecule library using random primers (i.e., primers with random base sequences) comprises annealing one or more random primer-adapters to template RNA molecules comprising a poly (A) tail, thereby generating annealed RNA molecules. In step (b) of the method, the one or more annealed RNA molecules are mixed, in the presence of nucleotides, with one or more acceptor-adapters and a hybrid reverse transcriptase comprising a dsDNA binding protein, or a fragment of the dsDNA binding protein. In step (c) of the method, the hybrid reverse transcriptase is allowed to transcribe the annealed RNA molecule without thermal cycling to produce a cDNA molecule library. In some embodiments, steps (a)-(c) are performed in a single reaction vessel. In certain embodiments, step (a) or step (b) further comprises adding hot start thermostable polymerase. In some embodiments, the template RNA molecules do not comprise a poly (A) tail.

In methods in which the hybrid reverse transcriptase comprises a dsDNA binding protein, the hybrid reverse transcriptase comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 40, SEQ ID NO: 41, SEQ ID NO: 42, or SEQ ID NO: 43 or a sequence at least 75% identical to SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 40, SEQ ID NO: 41, SEQ ID NO: 42, or SEQ ID NO: 43.

Methods in which Hybrid Reverse Transcriptase Comprises a ssDNA Binding Protein

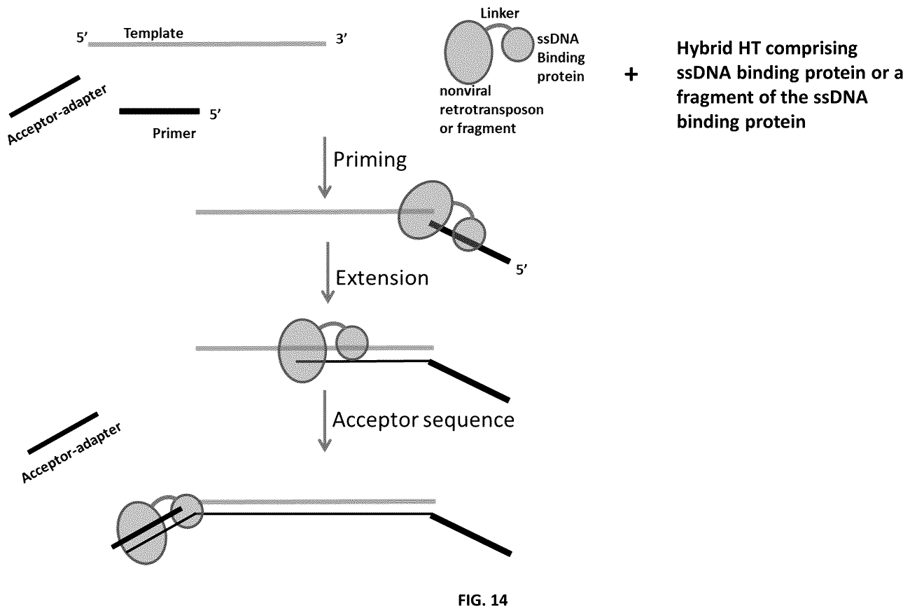

In some embodiments (FIG. 14), a method of preparing a cDNA molecule comprises contacting a template RNA molecule and free nucleotides with a primer that is not complementary (or has limited complementarity, i.e., 1 to 3 base pair complementarity) to the template RNA molecule, an acceptor-adapter; and a hybrid reverse transcriptase comprising a ssDNA binding protein or a fragment of the ssDNA binding protein. The hybrid reverse transcriptase is then allowed to transcribe the template RNA molecule under conditions effective for producing a cDNA molecule complementary to the template RNA molecule and, optionally, to the acceptor-adapter. An advantage to this method and subsequent methods in which the hybrid reverse transcriptase comprises a ssDNA binding protein is that template RNA dephosphorylation and poly (A) tailing steps are absent.

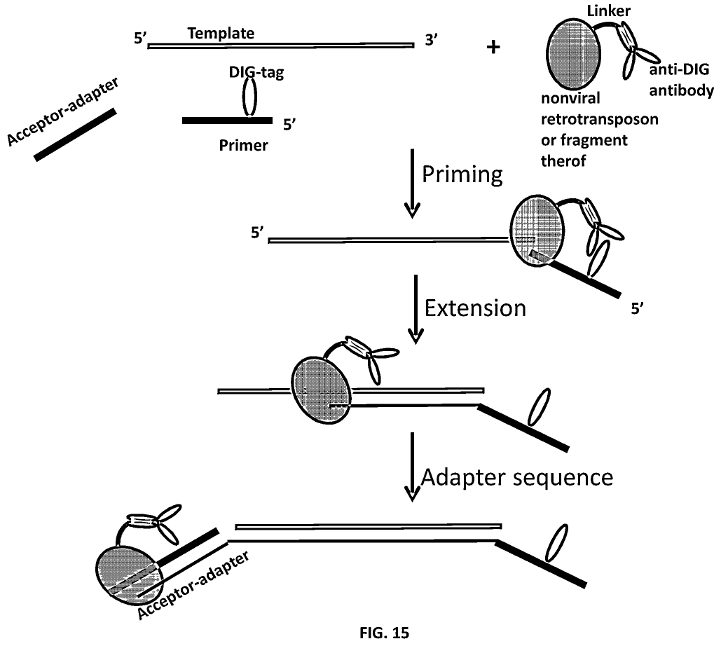

In certain embodiments, the primer-adapter comprises ssDNA or ssRNA. In some embodiments in which the hybrid reverse transcriptase is a fusion protein (FIG. 15), the ssDNA binding protein is an anti-Digoxigenin antibody and the primer-adapter comprises a Digoxigenin-tag (DIG-tag). During the transcription step of the method, the anti-digoxigenin antibody binds non-covalently to the DIG-tag in the primer-adapter, and may enhance the ability of the hybrid reverse transcriptase to transcribe the template RNA molecule.

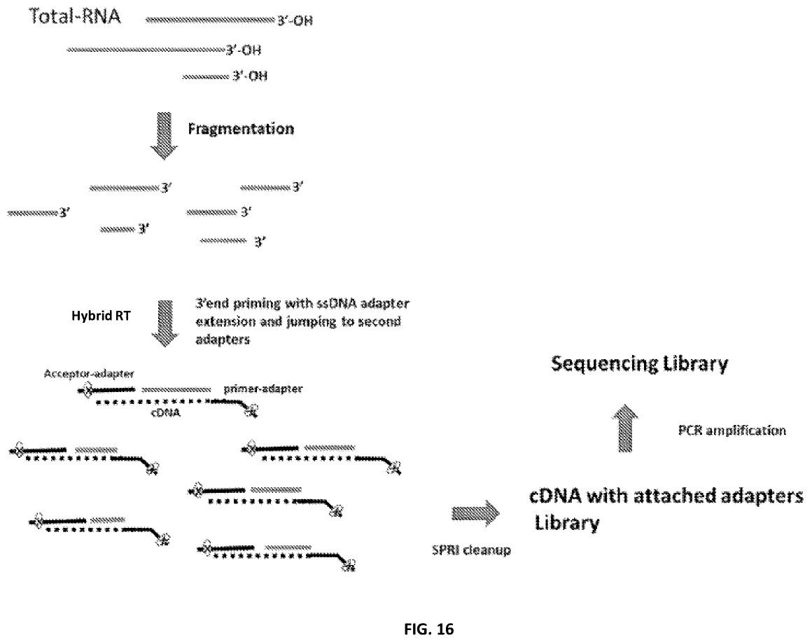

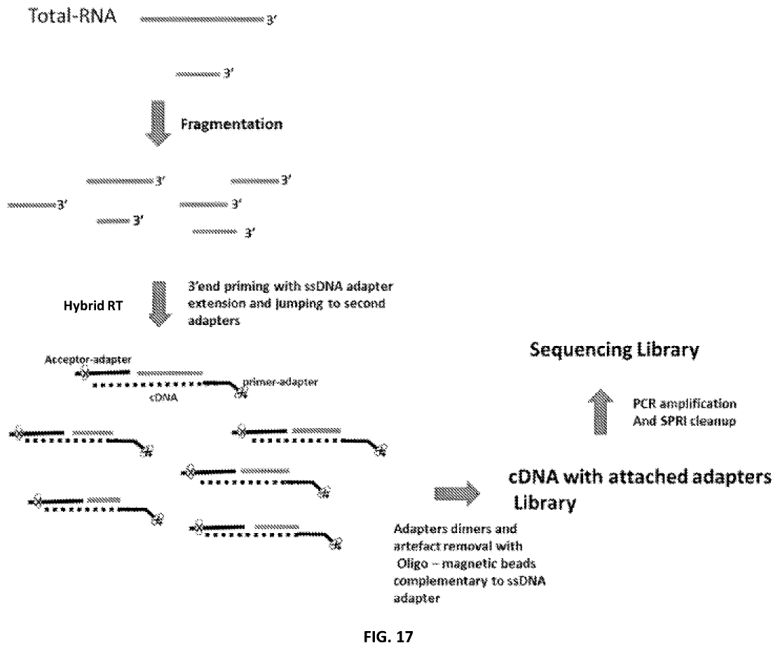

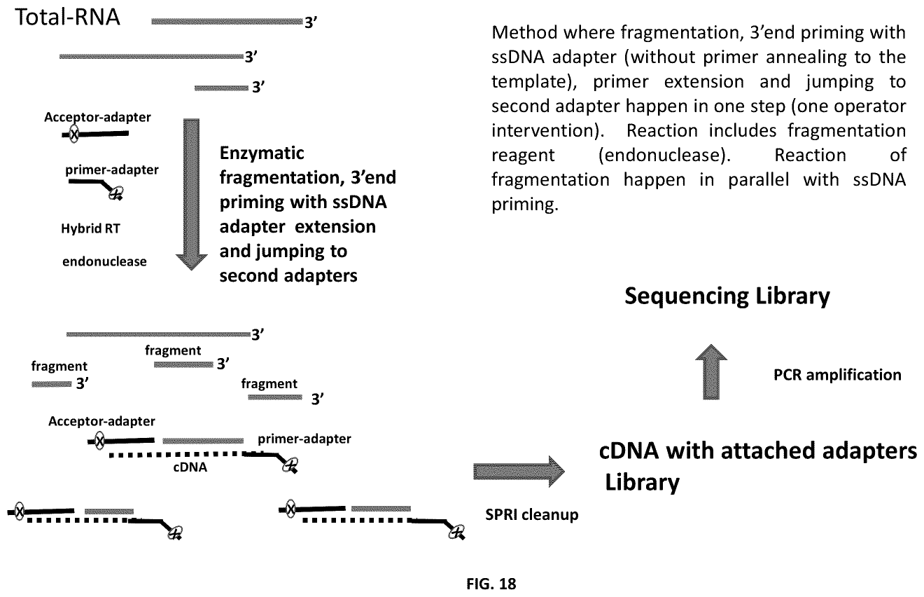

In certain embodiments (FIG. 16), step (a) of a method of preparing a cDNA molecule library comprises fragmenting a template RNA molecule to produce RNA fragments. In step (b) of the method, the RNA fragments and free nucleotides are contacted with a primer-adapter that is not complementary to the RNA fragments, an acceptor-adapter, and a hybrid reverse transcriptase comprising a ssDNA binding protein or a fragment of the ssDNA binding protein. The hybrid reverse transcriptase is then allowed to transcribe the RNA fragments under conditions effective to produce a cDNA molecule library in step (c) of the method. In some embodiments, the method further comprises removing one or more non-annealed primer-adapter after the last step by, for example, size fractionation via electrophoresis or chromatography. In some embodiments, one or more non-annealed primer-adapters are removed after the last step with an immobilized poly A oligonucleotide that hybridizes to a poly T sequence at the 3′end of the primer-adapter (FIG. 17). Solid phase reverse immobilization (SPRI) can also be used in place of or after the immobilized poly A oligonucleotide are used to remove the non-annealed primer-adapter artifacts. In some embodiments, steps (a)-(c) are combined into one step by further including an endonuclease which fragments the template RNA (FIG. 18). In certain embodiments, any or all of the steps are performed in a partition.

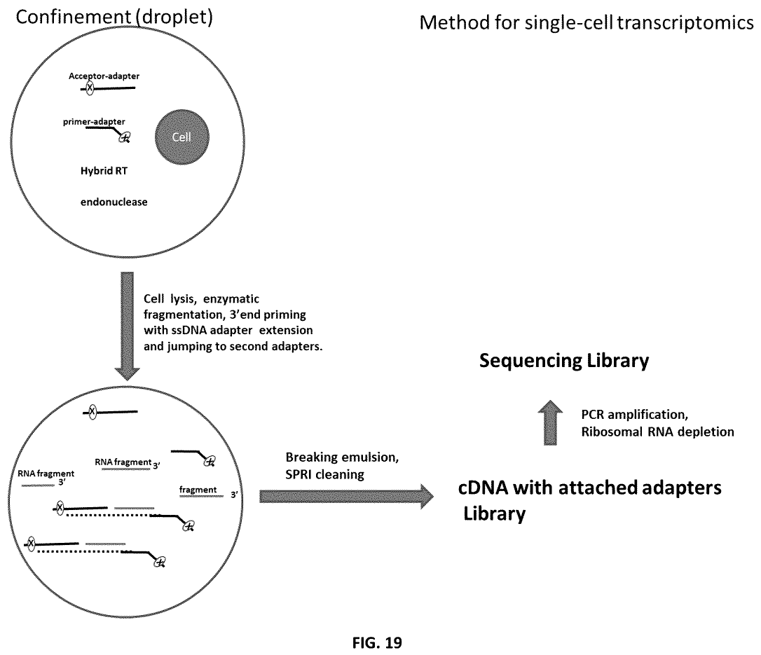

The hybrid reverse transcriptases can also be used in methods performed in a partition. In some embodiments (FIG. 19), step (a) of a method of preparing a cDNA molecule library comprises providing a partition comprising a cell comprising template RNA molecules, nucleotides, a primer-adapter that is not complementary to the template RNA molecules, an acceptor-adapter, an endonuclease, and a hybrid reverse transcriptase comprising a non-retroviral retrotransposon, or a fragment of the non-retroviral retrotransposon having reverse transcriptase activity, joined to a ssDNA binding protein. In the partition, template RNA molecules are released from the cell (step (b)), the RNA molecules are fragmented to form RNA fragments (step (c)), and the hybrid reverse transcriptase is allowed to transcribe the RNA fragments under conditions effective to produce a cDNA molecule library (step(d)).

The term “partitioning” or “partitioned” refers to separating an aqueous solution having one or more of a sample and reactants into a plurality of portions, or “partitions.” Partitions can be solid or fluid. In some embodiments, a partition is a solid partition, e.g., a microchannel. In some embodiments, a partition is a fluid partition (e.g., a droplet). In some embodiments, a fluid partition is a mixture of immiscible fluids (e.g., water and oil). In some embodiments, a fluid partition is an aqueous droplet that is surrounded by an immiscible carrier fluid (e.g., oil).

The partition can include any of a number of types of partitions, including solid partitions (e.g., wells or tubes) and fluid partitions (e.g., aqueous phase or droplet within an oil phase). In some embodiments, the partitions are droplets. In some embodiments, the partitions are microchannels. Methods and compositions for partitioning a sample are described, for example, in published patent application WO 2010/036352 and issued U.S. Pat. No. 9,156,010, the entire content of each of which is incorporated by reference herein.

In an embodiment, the droplet is formed by flowing an oil phase through an aqueous phase. The oil for the oil phase may be synthetic or naturally occurring. In some embodiments, the oil comprises carbon and/or silicon. In some embodiments, the oil comprises hydrogen and/or fluorine. Exemplary oils include, but are not limited to, silicone oil, mineral oil, fluorocarbon oil, vegetable oil, or a combination thereof.

In some embodiments, the droplet is formed by flowing an oil phase through an aqueous phase having a sample (e.g., a cell) and one or more components (e.g., reagents) that are used to prepare a cDNA library. In some embodiments, the one or more components used to prepare the cDNA library in the aqueous droplet are soluble and/or miscible in water including, but not limited to, one or more salts, buffering agents, reagents (e.g., nucleotides, a primer-adapter, an acceptor-adapter, an endonuclease, and a hybrid reverse transcriptase), surfactants, and/or whatever additional components may be necessary for a desired reaction(s) that may be intended to occur within a formed droplet. All such additional components may be selected to be compatible with the desired reaction.

In methods in which the hybrid reverse transcriptase comprises a ssDNA binding protein, the hybrid reverse transcriptase comprises SEQ ID NO: 14, SEQ ID NO: 15, SEQ ID NO: 16, SEQ ID NO: 17, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 47, SEQ ID NO: 98, SEQ ID NO: 99, or SEQ ID NO: 100, or a sequence at least 75% identical to SEQ ID NO: 14, SEQ ID NO: 15, SEQ ID NO: 16, SEQ ID NO: 17, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 47, SEQ ID NO: 98, SEQ ID NO: 99, or SEQ ID NO: 100.

In some embodiments, the cDNA molecule or cDNA library is prepared in about 1-3 hours (e.g., 2 hours) or less.

In some embodiments, the method further comprises amplifying the cDNA molecule(s) by polymerase chain reaction (PCR), thereby forming one or more amplicons. PCR techniques are described in, for example, Walker-Daniels (2012) “Current PCR Methods,” Mater. Methods, Vol. 2, No. 119. PCR techniques that may be used to amplify the cDNA molecule(s) include, but are not limited to, RT-PCR (in which reverse transcription and PCR are performed in the same reaction vessel), real-time PCR, nested PCR, quantitative PCR, multiplexed PCR, digital PCR, and assembly PCR. In certain embodiments, the amplifying is performed at a temperature sufficient to inactivate the reverse transcriptase.

In some embodiments, the method further comprises adding a label (e.g., a fluorescent dye) to the template RNA molecule(s), thereby generating a labeled cDNA molecule (s). In some embodiments, the method further comprises sequencing the labeled cDNA molecule(s). Sequencing the labeled cDNA molecule(s) may be performed by sequencing methods including, but not limited to, Helioscope® single molecule sequencing, Nanopore DNA sequencing, Lynx Therapeutics' Massively Parallel Signature Sequencing (MPSS), 454 pyrosequencing, Single Molecule real time (RNAP) sequencing, Illumina (Solexa) sequencing, SOLiD sequencing, Ion Torrent, Ion semiconductor sequencing, Single Molecule SMRT® sequencing, Polony sequencing, DNA nanoball sequencing, and VisiGen Biotechnologies approach. Alternatively, determining the sequence of the labeled nucleic acid or any product thereof may use sequencing platforms, including, but not limited to, Genome Analyzer IN, HiSeq, and MiSeq offered by Illumina, Single Molecule Real Time (SMRT®) technology, such as the PacBio RS system offered by Pacific Biosciences (California) and the Solexa Sequencer, True Single Molecule Sequencing (tSMS®) technology such as the HeliScope® Sequencer offered by Helicos Inc. (Cambridge, Mass.). The sequencing reaction can occur on a solid or semi-solid support, in a gel, in an emulsion, on a surface, on a bead, in a drop, in a continuous follow, in a dilution, or in one or more physically separate volumes.

### Kits

Also provided are kits for preparing cDNA from RNA. In some embodiments, such kits include a hybrid reverse transcriptase according to embodiments. In some embodiments, the kits further include suitable buffers, substrates for DNA synthesis such as the deoxynucleotide triphosphates (e.g., dATP, dCTP, dGTP, and dTTP) either individually or collectively in a suitable solution, a terminal transferase in solution, primer-adapter RNA having a known nucleotide sequence for use as a reverse transcription primer to obtain a 3′ end of RNA, and acceptor-adapter RNA having a known nucleotide sequence to obtain a 5′ end of RNA. In certain embodiments, the kits also include reagents for PCR (e.g., PCR primers, polymerase, buffers) and/or reagents for sequencing (e.g., fluorescently labeled dNTPs, sequencing primers). Any combination of the kit components may be provided in solution or in lyophilized form. The kits may also include instructions for using the kit components.

Various non-limiting embodiments include:

1. A hybrid reverse transcriptase comprising a non-retroviral retrotransposon, or a fragment of the non-retroviral retrotransposon having reverse transcriptase activity, joined to a nucleic acid binding protein or a fragment of the nucleic acid binding protein that binds to nucleic acid.

2. The hybrid reverse transcriptase of embodiment 1, wherein the nucleic acid binding protein is a single stranded deoxyribonucleic acid (ssDNA) binding protein or a fragment of a ssDNA binding protein.

3. The hybrid reverse transcriptase of embodiment 1, wherein the nucleic acid binding protein is a double stranded deoxyribonucleic acid (dsDNA) binding protein or a fragment of a dsDNA binding protein.

4. The hybrid reverse transcriptase of embodiment 1, wherein the nucleic acid binding protein is a nucleic acid tag binding protein.

5. The hybrid reverse transcriptase of any one of the preceding embodiments, wherein the non-retroviral retrotransposon, or the fragment of the non-retroviral retrotransposon, is an R2 reverse transcriptase or a fragment of the R2 reverse transcriptase, respectively.

6. The hybrid reverse transcriptase of embodiment 5, wherein the R2 reverse transcriptase comprises domains from two or more arthropods.

7. The hybrid reverse transcriptase of any one of embodiments 1-6, wherein the non-retroviral retrotransposon, or the fragment of the non-retroviral retrotransposon, is optionally joined via a linker to the nucleic acid binding protein or a fragment of the nucleic acid binding protein.

8. The hybrid reverse transcriptase of any one of embodiments 1-6, wherein the non-retroviral retrotransposon, or the fragment of the non-retroviral retrotransposon, is joined via a linker to the nucleic acid binding protein or a fragment of the nucleic acid binding protein.

9. The hybrid reverse transcriptase of embodiment 7 or 8, wherein the linker lacks a secondary structure.

10. The hybrid reverse transcriptase of embodiment 8 or 9, wherein the linker is selected from the group consisting of VGTVGTGGGSGGASTAL (SEQ ID NO: 101), VGTVGTGGGSEAAAKGGASTAL (SEQ ID NO: 102), VGTGGGSEAAAKGGASTAL (SEQ ID NO: 103), VGTGGGSGGGEAAAKEAAAKSGGGS (SEQ ID NO: 104), VGTGGGSGGGEAAAKEAAAKSGGGSA (SEQ ID NO: 105), VGTGGGSGGGTGGGS (SEQ ID NO: 106), and VGTGGGSGGGTGGGSA (SEQ ID NO: 107), (GGGS)ₙ(SEQ ID NO: 108), (GGS)ₙ, (GGGGS)ₙ (SEQ ID NO: 109), and (EAAAK)ₙ (SEQ ID NO: 110) and n is 1, 2, 3, 4, or 5.

11. The hybrid reverse transcriptase of embodiment 2, wherein the nucleic acid binding protein is Sulfolobus SSB, a fragment of Sulfolobus SSB, or an anti-Digoxigenin antibody.

12. The hybrid reverse transcriptase of embodiment 3, wherein the nucleic acid binding protein is Sso7d, a fragment of Sso7d, Cren7, or a fragment of Cren7.

13. The hybrid reverse transcriptase of any one of the preceding embodiments, wherein the nucleic acid binding protein, or the fragment of the nucleic acid binding protein, is joined to the N-terminus or C-terminus of the non-retroviral retrotransposon or the fragment of the non-retroviral retrotransposon.

14. The hybrid reverse transcriptase of any one of the preceding embodiments, wherein the nucleic acid binding protein is two or more nucleic acid binding proteins or fragments of two or more nucleic acid binding proteins and the two or more nucleic acid binding proteins or fragments of two or more nucleic acid binding proteins are identical or non-identical.

15. The hybrid reverse transcriptase of embodiment 14, wherein the two or more nucleic acid binding proteins or fragments of two or more nucleic acid binding proteins are in sequential or random order.

16. The hybrid reverse transcriptase of any one of the preceding embodiments, wherein the non-retroviral retrotransposon, or the fragment of the non-retroviral retrotransposon, is covalently joined to the nucleic acid binding protein or the fragment of the nucleic acid binding protein.

17. A hybrid reverse transcriptase comprising two or more non-retroviral retrotransposons, or two or more fragments of the non-retroviral retrotransposon having reverse transcriptase activity, optionally joined by a linker.

18. The hybrid reverse transcriptase of embodiment 17, wherein the two or more non-retroviral retrotransposons, or the two or more fragments of the non-retroviral retrotransposon having reverse transcriptase activity are joined by a linker.

19. The hybrid reverse transcriptase of embodiment 17 or 18, wherein the linker lacks a secondary structure.

20. The hybrid reverse transcriptase of any one of embodiments 17-19, wherein the linker is selected from the group consisting of VGTVGTGGGSGGASTAL (SEQ ID NO: 101), VGTVGTGGGSEAAAKGGASTAL (SEQ ID NO: 102), VGTGGGSEAAAKGGASTAL (SEQ ID NO: 103), VGTGGGSGGGEAAAKEAAAKSGGGS (SEQ ID NO: 104), VGTGGGSGGGEAAAKEAAAKSGGGSA (SEQ ID NO: 105), VGTGGGSGGGTGGGS (SEQ ID NO: 106), and VGTGGGSGGGTGGGSA (SEQ ID NO: 107), (GGGS)ₙ (SEQ ID NO: 108), (GGS)ₙ, (GGGGS)ₙ (SEQ ID NO: 109), and (EAAAK)ₙ (SEQ ID NO: 110) wherein n is 1, 2, 3, 4, or 5.

21. The hybrid reverse transcriptase of any one of embodiments 17-20, wherein the two or more non-retroviral retrotransposons, or the two or more fragments of the non-retroviral retrotransposon, are an R2 reverse transcriptase or a fragment of the R2 reverse transcriptase, respectively.

22. The hybrid reverse transcriptase of any one of embodiments 17-21, wherein the two or more non-retroviral retrotransposons, or the two or more fragments of the non-retroviral retrotransposon, are covalently joined.

23. The hybrid reverse transcriptase of any one of the preceding embodiments, further comprising a purification tag at an N-terminus or a C-terminus.

24. A hybrid reverse transcriptase comprising a non-retroviral retrotransposon having at least 75% sequence identity to SEQ ID NO: 1 joined to a nucleic acid binding protein or a fragment of the nucleic acid binding protein.

25. A hybrid reverse transcriptase comprising a fragment of a non-retroviral retrotransposon having at least 75% sequence identity to SEQ ID NO: 2 joined to a nucleic acid binding protein or a fragment of the nucleic acid binding protein.

26. A hybrid reverse transcriptase comprising SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 98, SEQ ID NO: 99, or SEQ ID NO: 100 or a sequence with at least 75% identity to SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 47, SEQ ID NO: 98, SEQ ID NO: 99, or SEQ ID NO: 100.

27. A nucleic acid construct comprising a polynucleotide sequence encoding the hybrid reverse transcriptase of any one of the preceding embodiments.

28. A vector comprising the nucleic acid construct of embodiment 27.

29. A host cell comprising a nucleic acid construct and/or a vector as defined in embodiment 27 or 28.

30. A method of preparing a cDNA molecule comprising:
(a) contacting a template RNA molecule and free nucleotides with:
   i. a primer that is complementary to the template RNA molecule;
   ii. an acceptor-adapter; and
   iii. a hybrid reverse transcriptase comprising a non-retroviral retrotransposon, or a fragment of the non-retroviral retrotransposon having reverse transcriptase activity, joined to a dsDNA binding protein or a fragment of a dsDNA binding protein; and
(b) allowing the hybrid reverse transcriptase to transcribe the template RNA molecule under conditions effective to produce a cDNA molecule complementary to the RNA molecule and, optionally, to the acceptor-adapter,
wherein the hybrid reverse transcriptase jumps to a 3′-end of the acceptor-adapter upon reaching a 5′ end of the RNA molecule.

31. A method of preparing a cDNA molecule library comprising:
(a) fragmenting a template RNA molecule to produce RNA fragments;
(b) removing a 2′, 3′-cyclic phosphate and a 3′-phosphate from the RNA fragments, thereby generating dephosphorylated RNA fragments;
(c) adding a poly-A tail to the dephosphorylated RNA fragments to form poly-A tailed RNA fragments;
(d) adding to the poly-A tailed RNA fragments:
   i. a primer-adapter comprising an oligo-T sequence;
   ii. an acceptor-adapter;
   iii. nucleotides; and
   iv. a hybrid reverse transcriptase comprising a non-retroviral retrotransposon, or a fragment of the non-retroviral retrotransposon having reverse transcriptase activity, joined to a dsDNA binding protein or a fragment of a dsDNA binding protein; and
(e) allowing the hybrid reverse transcriptase to transcribe the poly-A tailed RNA fragments under conditions effective to produce a cDNA molecule library,
wherein the hybrid reverse transcriptase jumps to a 3′-end of the acceptor-adapter upon reaching a 5′ end of the poly-A tailed RNA fragments.

32. The method of embodiment 30, wherein steps (b) and (c) are combined into one step.

33. A method of preparing a cDNA molecule library comprising:
(a) annealing a primer adapter comprising a poly (T) tail to a template RNA molecule comprising a poly (A) tail, thereby generating an annealed RNA molecule;
(b) mixing:
   i. the annealed RNA molecule;
   ii. an acceptor-adapter;
   iii. nucleotides; and
   iv. a hybrid reverse transcriptase comprising a non-retroviral retrotransposon, or a fragment of the non-retroviral retrotransposon having reverse transcriptase activity, joined to a dsDNA binding protein or a fragment of a dsDNA binding protein; and
(c) allowing the hybrid reverse transcriptase to transcribe the annealed RNA molecule at a temperature from about 12° C. to about 42° C. to produce a cDNA molecule library,
wherein the hybrid reverse transcriptase jumps to a 3′-end of the acceptor-adapter upon reaching a 5′ end of the annealed RNA molecule.

34. The method of embodiment 33, wherein the template RNA molecule is a messenger RNA molecule.

35. A method of preparing a cDNA molecule library comprising:
(a) annealing one or more random primer adapters to template RNA molecules comprising a poly (A) tail, thereby generating annealed RNA molecules;
(b) mixing:
   i. the one or more annealed RNA molecules;
   ii. one or more acceptor-adapters;
   iii. nucleotides; and
   iv. a hybrid reverse transcriptase comprising a non-retroviral retrotransposon, or a fragment of the non-retroviral retrotransposon having reverse transcriptase activity, joined to a dsDNA binding protein or a fragment of a dsDNA binding protein; and
(c) allowing the hybrid reverse transcriptase to transcribe the annealed RNA molecule without thermal cycling to produce a cDNA molecule library,
wherein the hybrid reverse transcriptase jumps to a 3′-end of the acceptor-adapter upon reaching a 5′ end of the annealed RNA molecules.

36. The method of embodiment 35, wherein the steps (a)-(c) are performed in a single reaction vessel.

37. The method of embodiment 35 or 36, wherein the template RNA molecules comprise messenger RNAs, ribosomal RNAs, transfer RNAs (tRNAs), micro RNAs, and/or long non-coding RNAs.

38. The method of any one of embodiments 30-37, wherein the hybrid reverse transcriptase comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 40, SEQ ID NO: 41, SEQ ID NO: 42, or SEQ ID NO: 43 or a sequence at least 75% identical to SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 40, SEQ ID NO: 41, SEQ ID NO: 42, or SEQ ID NO: 43.

39. A method of preparing a cDNA molecule comprising:
(a) contacting a template RNA molecule and free nucleotides with:
   i. a primer that is not complementary to the template RNA molecule;
   ii. an acceptor-adapter; and
   iii. a hybrid reverse transcriptase comprising a non-retroviral retrotransposon, or a fragment of the non-retroviral retrotransposon having reverse transcriptase activity, joined to a ssDNA binding protein or a fragment of a ssDNA binding protein; and
(b) allowing the hybrid reverse transcriptase to transcribe the template RNA molecule under conditions effective for producing a cDNA molecule complementary to the RNA molecule and, optionally, to the acceptor-adapter,
wherein the hybrid reverse transcriptase jumps to a 3′-end of the acceptor-adapter upon reaching a 5′ end of the RNA molecule.

40. The method of embodiment 39, wherein the primer comprises ssDNA or ssRNA.

41. The method of embodiment 39, wherein the ssDNA binding protein is an anti-Digoxigenin antibody and the primer comprises a Digoxigenin tag.

42. A method of preparing a cDNA molecule library comprising:
(a) fragmenting a template RNA molecule to produce RNA fragments;
(b) contacting the RNA fragments and free nucleotides, with:
   i. a primer-adapter that is not complementary to the RNA fragments;
   ii. an acceptor-adapter; and
   iii. a hybrid reverse transcriptase comprising a non-retroviral retrotransposon, or a fragment of the non-retroviral retrotransposon having reverse transcriptase activity, joined to a ssDNA binding protein or a fragment of a ssDNA binding protein; and
(c) allowing the hybrid reverse transcriptase to transcribe the RNA fragments under conditions effective to produce a cDNA molecule library,
wherein the hybrid reverse transcriptase jumps to a 3′-end of the acceptor-adapter upon reaching a 5′ end of the RNA fragments.

43. The method of embodiment 42, wherein steps (a)-(c) are combined into one step.

44. The method of any one of embodiments 30-43, wherein any or all of the steps are performed in a partition.

45. A method of preparing a cDNA molecule library comprising:
(a) providing a partition comprising:
   i. a cell comprising template RNA molecules;
   ii. nucleotides;
   iii. a primer adapter that is not complementary to the RNA molecules;
   iv. an acceptor-adapter;
   v. an endonuclease; and
   vi. a hybrid reverse transcriptase comprising a non-retroviral retrotransposon, or a fragment of the non-retroviral retrotransposon having reverse transcriptase activity, joined to a ssDNA binding protein or a fragment of a ssDNA binding protein;
in the partition:
(b) releasing template RNA molecules from the cell;
(c) fragmenting the template RNA molecules to form RNA fragments; and
(d) allowing the hybrid reverse transcriptase to transcribe the RNA fragments under conditions effective to produce a cDNA molecule library,
wherein the hybrid reverse transcriptase jumps to a 3′-end of the acceptor-adapter upon reaching a 5′ end of the dephosphorylated RNA fragments.

46. The method of any one of embodiments 39-45, wherein the hybrid reverse transcriptase comprises SEQ ID NO: 14, SEQ ID NO: 15, SEQ ID NO: 16, SEQ ID NO: 17, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 47, SEQ ID NO: 98, SEQ ID NO: 99, or SEQ ID NO: 100, or a sequence at least 75% identical to SEQ ID NO: 14, SEQ ID NO: 15, SEQ ID NO: 16, SEQ ID NO: 17, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 47, SEQ ID NO: 98, SEQ ID NO: 99, or SEQ ID NO: 100.

47. A hybrid reverse transcriptase comprising:
a non-retroviral retrotransposon, or a fragment of the non-retroviral retrotransposon having reverse transcriptase activity, in either case comprising a first binding motif; and
a nucleic acid binding protein, or a fragment of the nucleic acid binding protein, in either case comprising a second binding motif,
wherein the first binding motif is covalently joined to the second binding motif via protein ligation.

48. The hybrid reverse transcriptase of embodiment 47, wherein the nucleic acid binding protein is a single stranded deoxyribonucleic acid (ssDNA) binding protein or a fragment of the ssDNA binding protein.

49. The hybrid reverse transcriptase of embodiment 47, wherein the nucleic acid binding protein is a double stranded deoxyribonucleic acid (dsDNA) binding protein or a fragment of the dsDNA binding protein.

50. The hybrid reverse transcriptase of any one of embodiments 47-49, wherein a binding motif is located at a C terminus or an N-terminus of the non-retroviral retrotransposon, the fragment of the non-retroviral retrotransposon, the nucleic acid binding protein, or the fragment of the nucleic acid binding protein.

51. The hybrid reverse transcriptase of embodiment 50, wherein the binding motif is joined to the non-retroviral retrotransposon, the fragment of the non-retroviral retrotransposon, the nucleic acid binding protein, or the fragment of the nucleic acid binding protein via a linker.

52. The hybrid reverse transcriptase of embodiment 51, wherein the linker is selected from the group consisting of VGTVGTGGGSGGASTAL (SEQ ID NO: 101), VGTVGTGGGSEAAAKGGASTAL (SEQ ID NO: 102), VGTGGGSEAAAKGGASTAL (SEQ ID NO: 103), VGTGGGSGGGEAAAKEAAAKSGGGS (SEQ ID NO: 104), VGTGGGSGGGEAAAKEAAAKSGGGSA (SEQ ID NO: 105), VGTGGGSGGGTGGGS (SEQ ID NO: 106), and VGTGGGSGGGTGGGSA (SEQ ID NO: 107), (GGGS)ₙ(SEQ ID NO: 108), (GGS)ₙ, (GGGGS)ₙ (SEQ ID NO: 109), and (EAAAK)ₙ (SEQ ID NO: 110) wherein n is 1, 2, 3, 4, or 5.

53. The hybrid reverse transcriptase of embodiment 48, wherein the nucleic acid binding protein is Sulfolobus SSB or a fragment of Sulfolobus SSB.

54. The hybrid reverse transcriptase of embodiment 49, wherein the nucleic acid binding protein is Sso7d, a fragment of Sso7d, Cren7, or a fragment of Cren7.

55. The hybrid reverse transcriptase of any one of embodiments 47-54, further comprising a purification tag at an N-terminus or C-terminus of the non-retroviral retrotransposon, the fragment of the non-retroviral retrotransposon, the nucleic acid binding protein, or the fragment of the nucleic acid binding protein.

56. The hybrid reverse transcriptase of any one of embodiments 47-55, wherein the non-retroviral retrotransposons, or the fragment of the non-retroviral retrotransposon, is an R2 reverse transcriptase or a fragment of the R2 reverse transcriptase, respectively.

57. The hybrid reverse transcriptase of embodiment 56, wherein the R2 reverse transcriptase comprises domains from two or more arthropods.

58. A hybrid reverse transcriptase comprising:
a first non-retroviral retrotransposon, or a first fragment of the non-retroviral retrotransposon having reverse transcriptase activity, in either case comprising a first binding motif; and
a second non-retroviral retrotransposon, or a second fragment of the non-retroviral retrotransposon having reverse transcriptase activity, in either case comprising a second binding motif,
wherein the first binding motif is covalently joined to the second binding motif via protein ligation.

59. The hybrid reverse transcriptase of embodiment 58, wherein the first and second non-retroviral retrotransposons, or the first and second fragments of the non-retroviral retrotransposon, are an R2 reverse transcriptase or a fragment of the R2 reverse transcriptase, respectively.

60. The hybrid reverse transcriptase of any one of embodiments 47-59, wherein:
(a) the first binding motif comprises SEQ ID NO: 48, 49, or 50 or a sequence with at least 60% identity to 48, 49, or 50 and the second binding motif comprises SEQ ID NO: 51, 52, 53, 54, or 55 or a sequence with at least 60% identity to 51, 52, 53, 54, or 55; or
(b) the first binding motif comprises SEQ ID NO: 51, 52, 53, 54, or 55 or a sequence with at least 60% identity to 51, 52, 53, 54, or 55 and the second binding motif comprises SEQ ID NO: 48, 49, or 50 or a sequence with at least 60% identity to 48, 49, or 50.

61. The hybrid reverse transcriptase of any one of embodiments 47-59, wherein:
(a) the first binding motif comprises SEQ ID NO: 56, 57, or 58 or a sequence with at least 60% identity to 56, 57, or 58 and the second binding motif comprises SEQ ID NO: 59, 60, or 61 or a sequence with at least 60% identity to 59, 60, or 61; or
(b) the first binding motif comprises SEQ ID NO: 59, 60, or 61 or a sequence with at least 60% identity to 59, 60, or 61 and the second binding motif comprises SEQ ID NO: 56, 57, or 58 or a sequence with at least 60% identity to 56, 57, or 58.

62. The hybrid reverse transcriptase of any one of embodiments 47-59, wherein the first binding motif comprises a sortase recognition domain comprising the amino acid sequence: LPTGAA (SEQ ID NO: 62), LPTGGG (SEQ ID NO: 63), LPKTGG (SEQ ID NO: 64), LPETG (SEQ ID NO: 65), LPXTG (SEQ ID NO: 66) or LPXTG(X)n (SEQ ID NO: 67), where X is any amino acid, and n is 0, 1, 2, 3, 4, 5, 7, 8, 9, 10, in the range of 0-5 or 0-10, or any integer up to 100, or NPX1TX2 (SEQ ID NO: 68), where X1 is glutamine or lysine; X2 is asparagine or glycine; N is asparagine; P is proline and T is threonine, and the second binding motif comprises a sortase bridging domain comprising: Gly, (Gly)2, (Gly)3, (Gly)4, or (Gly)x, where x is an integer of 1-20.

63. A hybrid reverse transcriptase comprising a non-retroviral retrotransposon having at least 75% sequence identity to SEQ ID NO: 1 joined via protein ligation to a nucleic acid binding protein or a fragment of the nucleic acid binding protein.

64. A hybrid reverse transcriptase comprising a fragment of a non-retroviral retrotransposon having at least 75% sequence identity to SEQ ID NO: 2 joined via protein ligation to a nucleic acid binding protein or a fragment of the nucleic acid binding protein.

65. A pair of nucleic acid constructs comprising:
a) a first nucleic acid construct comprising a polynucleotide sequence encoding a non-retroviral retrotransposon, or a fragment of the non-retroviral retrotransposon having reverse transcriptase activity, fused to a first binding motif; and
b) a second nucleic acid construct comprising a polynucleotide sequence encoding a nucleic acid binding protein, or a fragment of the nucleic acid binding protein, fused to a second binding motif,
wherein the first binding motif and the second binding motif form a covalent bond via protein ligation when brought into contact with one another either spontaneously or with the help of an enzyme.

66. A pair of nucleic acid constructs comprising:
a) a first nucleic acid construct comprising a polynucleotide sequence encoding a first non-retroviral retrotransposon, or a first fragment of the non-retroviral retrotransposon having reverse transcriptase activity, fused to a first binding motif; and
b) a second nucleic acid construct comprising a polynucleotide sequence encoding a second non-retroviral retrotransposon, or a second fragment of the non-retroviral retrotransposon having reverse transcriptase activity, fused to a second binding motif, wherein the first binding motif and the second binding motif form a covalent bond via protein ligation when brought into contact with one another either spontaneously or with the help of an enzyme.

67. A vector comprising the nucleic acid constructs of embodiment 65 or 66.

68. A host cell comprising a nucleic acid constructs and/or a vector as defined in any one of embodiments 65-67.

69. A method of producing a hybrid reverse transcriptase, the method comprising contacting a non-retroviral retrotransposon, or a fragment of the non-retroviral retrotransposon having reverse transcriptase activity, in either case comprising a first binding motif, with a nucleic acid binding protein, or a fragment of the nucleic acid binding protein, in either case comprising a second binding motif, wherein the contacting is performed under conditions that allow the first binding motif to covalently join via protein ligation, either spontaneously or with the help of an enzyme, to the second binding motif.

70. A method of producing a hybrid reverse transcriptase, the method comprising contacting a first non-retroviral retrotransposon, or a first fragment of the non-retroviral retrotransposon having reverse transcriptase activity, in either case comprising a first binding motif, with a second non-retroviral retrotransposon, or a second fragment of the non-retroviral retrotransposon, in either case comprising a second binding motif, wherein the contacting is performed under conditions that allow the first binding motif to covalently join via protein ligation, either spontaneously or with the help of an enzyme, to the second binding motif.

71. A method of preparing a cDNA molecule comprising:
(a) contacting a template RNA molecule and free nucleotides with:
   i. a primer that is complementary to the template RNA molecule;
   ii. an acceptor-adapter; and
   iii. a hybrid reverse transcriptase comprising a non-retroviral retrotransposon, or a fragment of the non-retroviral retrotransposon having reverse transcriptase activity, in either case comprising a first binding motif and a dsDNA binding protein, or a fragment of the dsDNA binding protein, in either case comprising a second binding motif,
   wherein the first binding motif is covalently joined to the second binding motif via protein ligation; and
(b) allowing the hybrid reverse transcriptase to transcribe the template RNA molecule under conditions effective to produce a cDNA molecule complementary to the RNA molecule and, optionally, to the acceptor-adapter,
wherein the hybrid reverse transcriptase jumps to a 3′-end of the acceptor-adapter upon reaching a 5′ end of the RNA molecule.

72. A method of preparing a cDNA molecule library comprising:
(a) fragmenting a template RNA molecule to produce RNA fragments;
(b) removing a 2′, 3′-cyclic phosphate and a 3′-phosphate from the RNA fragments, thereby generating dephosphorylated RNA fragments;
(c) adding a poly-A tail to the dephosphorylated RNA fragments to form poly-A tailed RNA fragments;
(d) adding to the poly-A tailed RNA fragments:
   i. a primer-adapter comprising an oligo-T sequence;
   ii. an acceptor-adapter;
   iii. nucleotides; and
   iv. a hybrid reverse transcriptase comprising a non-retroviral retrotransposon, or a fragment of the non-retroviral retrotransposon having reverse transcriptase activity, in either case comprising a first binding motif and a dsDNA binding protein, or a fragment of the dsDNA binding protein, in either case comprising a second binding motif,
   wherein the first binding motif is covalently joined to the second binding motif via protein ligation; and
(e) allowing the hybrid reverse transcriptase to transcribe the poly-A tailed RNA fragments under conditions effective to produce a cDNA molecule library,
wherein the hybrid reverse transcriptase jumps to a 3′-end of the acceptor-adapter upon reaching a 5′ end of the poly-A tailed RNA fragments.

73. The method of embodiment 72, wherein steps (b) and (c) are combined into one step.

74. A method of preparing a cDNA molecule library comprising:
(a) annealing a primer adapter comprising a poly (T) tail to a template RNA molecule comprising a poly (A) tail, thereby generating an annealed RNA molecule;
(b) mixing:
   i. the annealed RNA molecule;
   ii. an acceptor-adapter;
   iii. nucleotides; and
   iv. a hybrid reverse transcriptase comprising a non-retroviral retrotransposon, or a fragment of the non-retroviral retrotransposon having reverse transcriptase activity, comprising a first binding motif and a dsDNA binding protein, or a fragment of the dsDNA binding protein, in either case comprising a second binding motif,
   wherein the first binding motif is covalently joined to the second binding motif via protein ligation; and
(c) allowing the hybrid reverse transcriptase to transcribe the annealed RNA molecule at a temperature from about 12° C. to about 42° C. to produce a cDNA molecule library,
wherein the hybrid reverse transcriptase jumps to a 3′-end of the acceptor-adapter upon reaching a 5′ end of the annealed RNA molecule.

75. The method of embodiment 74, wherein the template RNA molecule is a messenger RNA molecule.

76. A method of preparing a cDNA molecule library comprising:
(a) annealing one or more random primer adapters to template RNA molecules comprising a poly (A) tail, thereby generating annealed RNA molecules;
(b) mixing:
   i. the one or more annealed RNA molecules;
   ii. one or more acceptor-adapters;
   iii. nucleotides; and
   iv. a hybrid reverse transcriptase comprising a non-retroviral retrotransposon, or a fragment of the non-retroviral retrotransposon having reverse transcriptase activity, in either case comprising a first binding motif and a dsDNA binding protein, or a fragment of the dsDNA binding protein, in either case comprising a second binding motif,
   wherein the first binding motif is covalently joined to the second binding motif via protein ligation; and
(c) allowing the hybrid reverse transcriptase to transcribe the annealed RNA molecule without thermal cycling to produce a cDNA molecule library,
wherein the hybrid reverse transcriptase jumps to a 3′-end of the acceptor-adapter upon reaching a 5′ end of the annealed RNA molecules.

77. The method of embodiment 76, wherein the steps (a)-(c) are performed in a single reaction vessel.

78. The method of embodiment 76 or 77, wherein the template RNA molecules comprise messenger RNAs, ribosomal RNAs, transfer RNAs (tRNAs), micro RNAs, and/or long non-coding RNAs.

79. A method of preparing a cDNA molecule comprising:
(a) contacting a template RNA molecule and free nucleotides with:
   i. a primer that is not complementary to the template RNA molecule;
   ii. an acceptor-adapter; and
   iii. a hybrid reverse transcriptase comprising a non-retroviral retrotransposon, or a fragment of the non-retroviral retrotransposon having reverse transcriptase activity, in either case comprising a first binding motif and a ssDNA binding protein, or a fragment of the ssDNA binding protein, in either case comprising a second binding motif,
   wherein the first binding motif is covalently joined to the second binding motif via protein ligation; and
(b) allowing the hybrid reverse transcriptase to transcribe the template RNA molecule under conditions effective for producing a cDNA molecule complementary to the RNA molecule and, optionally, to the acceptor-adapter,
wherein the hybrid reverse transcriptase jumps to a 3′-end of the acceptor-adapter upon reaching a 5′ end of the RNA molecule.

80. The method of embodiment 79, wherein the primer comprises ssDNA or ssRNA.

81. A method of preparing a cDNA molecule library comprising:
(a) fragmenting a template RNA molecule to produce RNA fragments;
(b) contacting the RNA fragments and free nucleotides with:
   i. a primer-adapter that is not complementary to the RNA fragments;
   ii. an acceptor-adapter; and
   iii. a hybrid reverse transcriptase comprising a non-retroviral retrotransposon, or a fragment of the non-retroviral retrotransposon having reverse transcriptase activity, in either case comprising a first binding motif and a ssDNA binding protein, or a fragment of the ssDNA binding protein, in either case comprising a second binding motif,
   wherein the first binding motif is covalently joined to the second binding motif via protein ligation; and
(c) allowing the hybrid reverse transcriptase to transcribe the RNA fragments under conditions effective to produce a cDNA molecule library,
wherein the hybrid reverse transcriptase jumps to a 3′-end of the acceptor-adapter upon reaching a 5′ end of the RNA fragments.

82. The method of embodiment 81, wherein steps (a)-(c) are combined into one step.

83. The method of any one of embodiments 71-82, wherein any or all of the steps are performed in a partition.

84. A method of preparing a cDNA molecule library comprising:
(a) providing a partition comprising:
   i. a cell comprising template RNA molecules;
   ii. nucleotides;
   iii. a primer adapter that is not complementary to the RNA molecules;
   iv. an acceptor-adapter;
   v. an endonuclease; and
   vi. a hybrid reverse transcriptase comprising a non-retroviral retrotransposon, or a fragment of the non-retroviral retrotransposon having reverse transcriptase activity, in either case comprising a first binding motif and a ssDNA binding protein, or a fragment of the ssDNA binding protein, in either case comprising a second binding motif,
   wherein the first binding motif is covalently joined to the second binding motif via protein ligation; and
in the partition:
(b) releasing template RNA molecules from the cell;
(c) fragmenting the template RNA molecules to form RNA fragments; and
(d) allowing the hybrid reverse transcriptase to transcribe the RNA fragments under conditions effective to produce a cDNA molecule library,
wherein the hybrid reverse transcriptase jumps to a 3′-end of the acceptor-adapter upon reaching a 5′ end of the dephosphorylated RNA fragments.

85. The method of any one of embodiments 71-84, wherein:
(a) the first binding motif comprises SEQ ID NO: 48, 49, or 50 or a sequence with at least 60% identity to 48, 49, or 50 and the second binding motif comprises SEQ ID NO: 51, 52, 53, 54, or 55 or a sequence with at least 60% identity to 51, 52, 53, 54, or 55; or
(b) the first binding motif comprises SEQ ID NO: 51, 52, 53, 54, or 55 or a sequence with at least 60% identity to 51, 52, 53, 54, or 55 and the second binding motif comprises SEQ ID NO: 48, 49, or 50 or a sequence with at least 60% identity to 48, 49, or 50.

86. The method of any one of embodiments 71-84, wherein:
(a) the first binding motif comprises SEQ ID NO: 56, 57, or 58 or a sequence with at least 60% identity to 56, 57, or 58 and the second binding motif comprises SEQ ID NO: 59, 60, or 61 or a sequence with at least 60% identity to 59, 60, or 61; or
(b) the first binding motif comprises SEQ ID NO: 59, 60, or 61 or a sequence with at least 60% identity to 59, 60, or 61 and the second binding motif comprises SEQ ID NO: 56, 57, or 58 or a sequence with at least 60% identity to 56, 57, or 58.

87. The method of any one of embodiments 71-84, wherein the first binding motif comprises a sortase recognition domain comprising the amino acid sequence: LPTGAA (SEQ ID NO: 62), LPTGGG (SEQ ID NO: 63), LPKTGG (SEQ ID NO: 64), LPETG (SEQ ID NO: 65), LPXTG (SEQ ID NO: 66) or LPXTG(X)n (SEQ ID NO: 67), where X is any amino acid, and n is 0, 1, 2, 3, 4, 5, 7, 8, 9, 10, in the range of 0-5 or 0-10, or any integer up to 100, or NPX1TX2 (SEQ ID NO: 68), where X1 is glutamine or lysine; X2 is asparagine or glycine; N is asparagine; P is proline and T is threonine, and the second binding motif comprises a sortase bridging domain comprising: Gly, (Gly)2, (Gly)3, (Gly)4, or (Gly)x, where x is an integer of 1-20.

88. A hybrid reverse transcriptase comprising:
a non-retroviral retrotransposon, or a fragment of the non-retroviral retrotransposon having reverse transcriptase activity, in either case comprising a biotinylated first acceptor peptide;
a nucleic acid binding protein, or a fragment of the nucleic acid binding protein, in either case comprising a biotinylated second acceptor peptide; and
a biotin-binding protein,
wherein the biotinylated first and second acceptor peptides are joined to the biotin-binding protein via non-covalent interactions.

89. The hybrid reverse transcriptase of embodiment 88, wherein the nucleic acid binding protein is a single stranded deoxyribonucleic acid (ssDNA) binding protein or a fragment of the ssDNA binding protein.

90. The hybrid reverse transcriptase of embodiment 88, wherein the nucleic acid binding protein is a double stranded deoxyribonucleic acid (dsDNA) binding protein, or a fragment of the dsDNA binding protein.

91. The hybrid reverse transcriptase of any one of embodiments 88-90, wherein a biotinylated acceptor peptide is located at a C terminus or an N-terminus of the non-retroviral retrotransposon, the fragment of the non-retroviral retrotransposon, the nucleic acid binding protein, or the fragment of the nucleic acid binding protein.

92. The hybrid reverse transcriptase of embodiment 91, wherein the biotinylated acceptor peptide is joined to the non-retroviral retrotransposon, the fragment of the non-retroviral retrotransposon, the nucleic acid binding protein, or the fragment of the nucleic acid binding protein via a linker.

93. The hybrid reverse transcriptase of embodiment 92, wherein the linker is selected from the group consisting of VGTVGTGGGSGGASTAL, VGTVGTGGGSEAAAKGGASTAL, VGTGGGSEAAAKGGASTAL, VGTGGGSGGGEAAAKEAAAKSGGGS, VGTGGGSGGGEAAAKEAAAKSGGGSA, VGTGGGSGGGTGGGS, VGTGGGSGGGTGGGSA, (GGGS)n, (GGS)n, (GGGGS)n, and (EAAAK)n wherein n is 1, 2, 3, 4, or 5.

94. The hybrid reverse transcriptase of embodiment 89, wherein the nucleic acid binding protein is Sulfolobus SSB or a fragment of Sulfolobus SSB.

95. The hybrid reverse transcriptase of embodiment 90, wherein the nucleic acid binding protein is Sso7d, a fragment of Sso7d, Cren7, or a fragment of Cren 7.

96. The hybrid reverse transcriptase of any one of embodiments 88-95, further comprising a purification tag at an N-terminus or C-terminus of the non-retroviral retrotransposon, the fragment of the non-retroviral retrotransposon, the nucleic acid binding protein, or the fragment of the nucleic acid binding protein.

97. The hybrid reverse transcriptase of any of embodiments 88-96, wherein the non-retroviral retrotransposons, or the fragment of the non-retroviral retrotransposon, is an R2 reverse transcriptase or a fragment of the R2 reverse transcriptase, respectively.

98. A hybrid reverse transcriptase comprising:
a first non-retroviral retrotransposon, or a first fragment of the non-retroviral retrotransposon having reverse transcriptase activity, in either case comprising a biotinylated first acceptor peptide; and
a second non-retroviral retrotransposon, or a second fragment of the non-retroviral retrotransposon having reverse transcriptase activity, in either case comprising a biotinylated second acceptor peptide; and
a biotin-binding protein,
wherein the biotinylated first and second acceptor peptides are joined to the biotin-binding protein via non-covalent interactions.

99. The hybrid reverse transcriptase of embodiment 98, wherein the first and second non-retroviral retrotransposons, or the first and second fragments of the non-retroviral retrotransposon, are an R2 reverse transcriptase or a fragment of the R2 reverse transcriptase, respectively.

100. The hybrid reverse transcriptase of any one of embodiments 88-99, wherein the biotinylated acceptor peptide comprises an acceptor peptide selected from the group consisting of SEQ ID NO: 85, 86, 87, 88, and 89.

101. A hybrid reverse transcriptase comprising a non-retroviral retrotransposon having at least 75% sequence identity to SEQ ID NO: 1 and a nucleic acid binding protein, or a fragment of the nucleic acid binding protein, non-covalently joined to a biotin-binding protein.

102. A hybrid reverse transcriptase comprising a fragment of a non-retroviral retrotransposon having at least 75% sequence identity to SEQ ID NO: 2 and a nucleic acid binding protein, or a fragment of the nucleic acid binding protein, non-covalently joined to a biotin-binding protein.

103. The hybrid reverse transcriptase of any one of embodiments 88-102, wherein the biotin-binding protein is streptavidin, traptavidin, or neutravidin.

104. A pair of nucleic acid constructs comprising:
a) a first nucleic acid construct comprising a polynucleotide sequence encoding a non-retroviral retrotransposon, or a fragment of the non-retroviral retrotransposon having reverse transcriptase activity, fused to a first acceptor peptide; and
b) a second nucleic acid construct comprising a polynucleotide sequence encoding a nucleic acid binding protein, or a fragment of the nucleic acid binding protein, fused to a second acceptor peptide,
wherein the first and second acceptor peptides are biotinylated in the presence of biotin, a biotin ligase, and ATP to form biotinylated first and second acceptor peptides; and
wherein the biotinylated first and second acceptor peptides bind to a biotin-binding protein via non-covalent interactions when brought into contact with one another.

105. A pair of nucleic acid constructs comprising:
a) a first nucleic acid construct comprising a polynucleotide sequence encoding a first non-retroviral retrotransposon, or a first fragment of the non-retroviral retrotransposon having reverse transcriptase activity, fused to a first acceptor peptide; and
b) a second nucleic acid construct comprising a polynucleotide sequence encoding a second non-retroviral retrotransposon, or a second fragment of the non-retroviral retrotransposon having reverse transcriptase activity, fused to a second acceptor peptide,
wherein the first and second acceptor peptides are biotinylated in the presence of biotin, a biotin ligase, and ATP to form biotinylated first and second acceptor peptides; and
wherein the biotinylated first and second acceptor peptides bind to a biotin-binding protein via non-covalent interactions when brought into contact with one another.

106. A vector comprising the nucleic acid constructs of embodiment 104 or 105.

107. A host cell comprising a nucleic acid constructs and/or a vector as defined in any one of embodiments 104-106.

108. A method of producing a hybrid reverse transcriptase, the method comprising contacting a non-retroviral retrotransposon, or a fragment of the non-retroviral retrotransposon having reverse transcriptase activity, in either case comprising a first acceptor peptide and a nucleic acid binding protein, or a fragment of the nucleic acid binding protein, in either case comprising a second acceptor peptide, with biotin in the presence of biotin ligase and ATP to form biotinylated first and second acceptor peptides; and
contacting the biotinylated first and second acceptor peptides with a biotin-binding protein to allow the biotinylated first and second biotin acceptor peptides to bind to the biotin-binding protein via non-covalent interactions.

109. A method of producing a hybrid reverse transcriptase, the method comprising contacting a first non-retroviral retrotransposon, or a first fragment of the non-retroviral retrotransposon having reverse transcriptase activity, in either case comprising a first acceptor peptide and a second non-retroviral retrotransposon, or a second fragment of the non-retroviral retrotransposon, in either case comprising a second acceptor peptide with biotin in the presence of biotin ligase and ATP to form biotinylated first and second acceptor peptides; and
contacting the biotinylated first and second acceptor peptides with a biotin-binding protein to allow the biotinylated first and second acceptor peptides to bind to the biotin-binding protein via non-covalent interactions.

110. A method of preparing a cDNA molecule comprising:
(a) contacting a template RNA molecule and free nucleotides with:
   i. a primer that is complementary to the template RNA molecule;
   ii. an acceptor-adapter; and
   iii. a hybrid reverse transcriptase comprising a non-retroviral retrotransposon, or a fragment of the non-retroviral retrotransposon having reverse transcriptase activity, in either case comprising a biotinylated first acceptor peptide and a dsDNA binding protein, or a fragment of the dsDNA binding protein, in either case comprising a biotinylated second acceptor peptide,
   wherein the biotinylated first and second acceptor peptides are joined to a biotin-binding protein via non-covalent interactions; and
(b) allowing the hybrid reverse transcriptase to transcribe the template RNA molecule under conditions effective to produce a cDNA molecule complementary to the RNA molecule and, optionally, to the acceptor-adapter,
wherein the hybrid reverse transcriptase jumps to a 3′-end of the acceptor-adapter upon reaching a 5′ end of the RNA molecule.

111. A method of preparing a cDNA molecule library comprising:
(a) fragmenting a template RNA molecule to produce RNA fragments;
(b) removing a 2′, 3′-cyclic phosphate and a 3′-phosphate from the RNA fragments, thereby generating dephosphorylated RNA fragments;
(c) adding a poly-A tail to the dephosphorylated RNA fragments to form poly-A tailed RNA fragments;
(d) adding to the poly-A tailed RNA fragments:
   i. a primer-adapter comprising an oligo-T sequence;
   ii. an acceptor-adapter;
   iii. nucleotides; and
   iv. a hybrid reverse transcriptase comprising a non-retroviral retrotransposon, or a fragment of the non-retroviral retrotransposon having reverse transcriptase activity, in either case comprising a biotinylated first acceptor peptide and a dsDNA binding protein, or a fragment of the dsDNA binding protein, in either case comprising a biotinylated second acceptor peptide,
   wherein the biotinylated first and second acceptor peptides are joined to a biotin-binding protein via non-covalent interactions; and
(e) allowing the hybrid reverse transcriptase to transcribe the poly-A tailed RNA fragments under conditions effective to produce a cDNA molecule library,
wherein the hybrid reverse transcriptase jumps to a 3′-end of the acceptor-adapter upon reaching a 5′ end of the poly-A tailed RNA fragments.

112. The method of embodiment 111, wherein steps (b) and (c) are combined into one step.

113. A method of preparing a cDNA molecule library comprising:
(a) annealing a primer adapter comprising a poly (T) tail to a template RNA molecule comprising a poly (A) tail, thereby generating an annealed RNA molecule;
(b) mixing:
   i. the annealed RNA molecule;
   ii. an acceptor-adapter;
   iii. nucleotides; and
   iv. a hybrid reverse transcriptase comprising a non-retroviral retrotransposon, or a fragment of the non-retroviral retrotransposon having reverse transcriptase activity, in either case comprising a biotinylated first acceptor peptide and a dsDNA binding protein, or a fragment of the dsDNA binding protein, in either case comprising a biotinylated second acceptor peptide,
   wherein the biotinylated first and second acceptor peptides are joined to a biotin-binding protein via non-covalent interactions; and
(c) allowing the hybrid reverse transcriptase to transcribe the annealed RNA molecule at a temperature from about 12° C. to about 42° C. to produce a cDNA molecule library,
wherein the hybrid reverse transcriptase jumps to a 3′-end of the acceptor-adapter upon reaching a 5′ end of the annealed RNA molecule.

114. The method of embodiment 113, wherein the template RNA molecule is a messenger RNA molecule.

115. A method of preparing a cDNA molecule library comprising:
(a) annealing one or more random primer adapters to template RNA molecules comprising a poly (A) tail, thereby generating annealed RNA molecules;
(b) mixing:
   i. the one or more annealed RNA molecules;
   ii. one or more acceptor-adapters;
   iii. nucleotides; and
   iv. a hybrid reverse transcriptase comprising a non-retroviral retrotransposon, or a fragment of the non-retroviral retrotransposon having reverse transcriptase activity, in either case comprising a biotinylated first acceptor peptide and a dsDNA binding protein, or a fragment of the dsDNA binding protein, in either case comprising a biotinylated second acceptor peptide,
   wherein the biotinylated first and second acceptor peptides are joined to a biotin-binding protein via non-covalent interactions; and
(c) allowing the hybrid reverse transcriptase to transcribe the annealed RNA molecule without thermal cycling to produce a cDNA molecule library,
wherein the hybrid reverse transcriptase jumps to a 3′-end of the acceptor-adapter upon reaching a 5′ end of the annealed RNA molecules.

116. The method of embodiment 115, wherein the steps (a)-(c) are performed in a single reaction vessel.

117. The method of embodiment 115 or 116, wherein the template RNA molecules comprise messenger RNAs, ribosomal RNAs, transfer RNAs (tRNAs), micro RNAs, and/or long non-coding RNAs.

118. A method of preparing a cDNA molecule comprising:
(a) contacting a template RNA molecule and free nucleotides with:
   i. a primer that is not complementary to the template RNA molecule;
   ii. an acceptor-adapter; and
   iii. a hybrid reverse transcriptase comprising a non-retroviral retrotransposon, or a fragment of the non-retroviral retrotransposon having reverse transcriptase activity, in either case comprising a biotinylated first acceptor peptide and a ssDNA binding protein, or a fragment of the ssDNA binding protein, in either case comprising a biotinylated second acceptor peptide,
   wherein the biotinylated first and second acceptor peptides are joined to a biotin-binding protein via non-covalent interactions; and
(b) allowing the hybrid reverse transcriptase to transcribe the template RNA molecule under conditions effective for producing a cDNA molecule complementary to the RNA molecule and, optionally, to the acceptor-adapter,
wherein the hybrid reverse transcriptase jumps to a 3′-end of the acceptor-adapter upon reaching a 5′ end of the RNA molecule.

119. The method of embodiment 118, wherein the primer comprises ssDNA or ssRNA.

120. A method of preparing a cDNA molecule library comprising:
(a) fragmenting a template RNA molecule to produce RNA fragments;
(b) contacting the RNA fragments and free nucleotides with:
   i. a primer-adapter that is not complementary to the RNA fragments;
   ii. an acceptor-adapter; and
   iii. a hybrid reverse transcriptase comprising a non-retroviral retrotransposon, or a fragment of the non-retroviral retrotransposon having reverse transcriptase activity, in either case comprising a biotinylated first acceptor peptide and a ssDNA binding protein, or a fragment of the ssDNA binding protein, in either case comprising a biotinylated second acceptor peptide,
   wherein the biotinylated first and second acceptor peptides are joined to a biotin-binding protein via non-covalent interactions; and
(c) allowing the hybrid reverse transcriptase to transcribe the RNA fragments under conditions effective to produce a cDNA molecule library,
wherein the hybrid reverse transcriptase jumps to a 3′-end of the acceptor-adapter upon reaching a 5′ end of the RNA fragments.

121. The method of embodiment 120, wherein steps (a)-(c) are combined into one step.

122. The method of any one of embodiments 110-121, wherein any or all of the steps are performed in a partition.

123. A method of preparing a cDNA molecule library comprising:
(a) providing a partition comprising:
   i. a cell comprising template RNA molecules;
   ii. nucleotides;
   iii. a primer adapter that is not complementary to the RNA molecules;
   iv. an acceptor-adapter;
   v. an endonuclease; and
   vi. a hybrid reverse transcriptase comprising a non-retroviral retrotransposon, or a fragment of the non-retroviral retrotransposon having reverse transcriptase activity, in either case comprising a biotinylated first acceptor peptide and a ssDNA binding protein, or a fragment of the ssDNA binding protein, in either case comprising a biotinylated second acceptor peptide,
   wherein the biotinylated first and second acceptor peptides are joined to a biotin-binding protein via non-covalent interactions; and
in the partition:
(b) releasing template RNA molecules from the cell;
(c) fragmenting the template RNA molecules to form RNA fragments; and
(d) allowing the hybrid reverse transcriptase to transcribe the RNA fragments under conditions effective to produce a cDNA molecule library,
wherein the hybrid reverse transcriptase jumps to a 3′-end of the acceptor-adapter upon reaching a 5′ end of the dephosphorylated RNA fragments.

124. The method of any one of embodiments 110-123, wherein the biotinylated acceptor peptide comprises an acceptor peptide selected from the group consisting of SEQ ID NO: 85, 86, 87, 88, and 89.

125. The method of any one of embodiments 30-45, 71-87, or 110-124, further comprising removing one or more non-annealed primer adapter after the last step.

126. The method of embodiment 125, wherein the one or more non-annealed primer-adapter is removed with an immobilized poly A oligo.

127. The method of any one of embodiments 30-45, 71-87, or 110-124, wherein the jumping of the hybrid reverse transcriptase to the 3′-end of the acceptor-adapter is independent of sequence identity between the template RNA molecule(s) and the acceptor-adapter.

128. The method of any one of embodiments 30-45, 71-87, or 110-124, wherein the cDNA molecule or cDNA library is prepared in about 2 hours or less.

129. The method of any one of embodiments 30-45, 71-87, or 110-124, further comprising amplifying the cDNA molecule(s) by polymerase chain reaction, thereby forming one or more amplicons.

130. The method of embodiment 129, wherein the amplifying is performed at a temperature sufficient to inactivate the reverse transcriptase.

131. The method of embodiment 128 or 129, wherein producing and amplifying the cDNA molecule(s) is performed in the same reaction vessel.

132. The method of embodiment 129 or 130, further comprising adding a label to the template RNA molecule(s), thereby generating a labeled cDNA molecule(s).

133. The method of embodiment 132, further comprising sequencing the labeled cDNA molecule(s).

134. The method of any one of embodiments 30-45, 71-87, or 110-124, wherein the acceptor-adapter, the primer adapter, or both the acceptor-adapter and primer adapter comprise a nucleotide analogue that stops the reverse transcription by the hybrid reverse transcriptase.

135. The method of embodiment 134, wherein the nucleotide analogue is at the 5′ end of the acceptor-adapter.

136. The method of embodiment 134, wherein the nucleotide analogue is at the 5′ end of the primer adapter.

137. The method of any one of embodiments 30-45, 71-87, or 110-124, wherein the acceptor-adapter comprises a 3′-dideoxynucleotide.

138. The method of any one of embodiments 30-45, 71-87, or 110-124, wherein the non-retroviral retrotransposon comprises SEQ ID NO: 1 or a sequence with at least 75% identity to SEQ ID NO: 1 joined to a nucleic acid binding protein, or a fragment of the nucleic acid binding protein.

139. The method of any one of embodiments 30-45, 71-87, or 110-124, wherein the fragment of the non-retroviral retrotransposon comprises SEQ ID NO: 2 or a sequence with at least 75% sequence identity to SEQ ID NO: 2 joined to a nucleic acid binding protein, or a fragment of the nucleic acid binding protein.

140. The method of embodiment 38, 48, 71-87, 110-124, wherein the hybrid reverse transcriptase comprises at least one improved property selected from the group consisting of higher processivity, longer shelf life, higher strand displacement, higher end-to-end template jumping, and higher affinity as compared to a non-retroviral retrotransposon, or a fragment of the non-retroviral retrotransposon, that is not joined to a nucleic acid binding protein, or a fragment of the nucleic acid binding protein.

141. The method of embodiment 140, wherein the processivity of the hybrid reverse transcriptase is about 20 or more nucleotides.

### EXAMPLES

The following examples are provided by way of illustration only and not by way of limitation. Those of skill in the art will readily recognize a variety of non-critical parameters that could be changed or modified to yield essentially the same or similar results.

### Example 1—Expression and Purification of Hybrid Reverse Transcriptases Comprising ΔR2 Enzyme Recombinantly Fused to Nucleic Acid Binding Protein

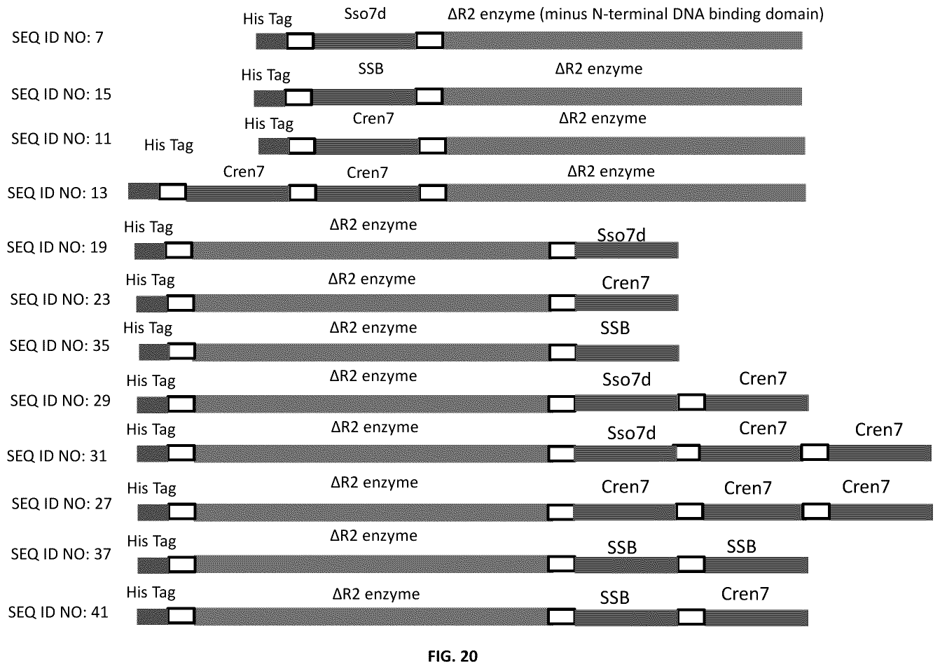

Expression vector pET-45b carrying the constructs shown in FIG. 20 were transformed into E. coli BL21 (DE3). For expression of each construct, a pre-culture was setup in 2 mL LB with 100 μM Corbenicillin and was grown for about 8-12 hours at room temperature. After about 8-12 hours, 550 μL of the pre-cultures were transferred to 25 mL auto-induction expression media (Overnight Express TB from Novagen) including 0.08 mg/ml Corbenicillin and shaker-incubated at 280-290 RPM at room temperature for 48 hours. Cells were harvested by centrifugation at 8000×g for 10 min at 4-8° C. The biomass-pellets were frozen at −20° C. for a minimum of 1 hour.

The pellets were re-suspended in 0.8 mL lysis buffer (0.8 ml lysis buffer per 10% of the biomass) and were incubated for 45 minutes at room temperature. Lysis buffer composition: 1×BugBuster (Millipore), 100 mM Sodium Phosphate, 0.2% Tween, 2.5 mM Tris (2-carboxyethyl) phosphine (TCEP), 6 μl Protease inhibitor mix (Roche), 0.1 mg/ml lysozyme, 0.5 ul DNaseI (2,000 units/ml, from NEB). After incubation, the lysates were mixed with equal volume (0.8 mL) of His-binding buffer (50 mM Sodium Phosphate pH 7.7, 1.5 M Sodium Chloride, 5 mM TCEP, 0.2% Tween, 0.03% Triton X-100, 10 mM Imidazole) and incubated at room temperature for 30 minutes. After incubation, the lysates were centrifuged at 13000×g for 40 minutes at 80° C. The pellets were each mixed with 250 ul of His-Affinity Gel (His-Spin Protein Miniprep by Zymo Research) per the manufacturer's protocol. After the binding step, for each pellet, the His-Affinity Gel was washed five times with Washing buffer (50 mM Sodium Phosphate pH7.7, 1.2M Sodium Chloride, 0.2% Tween, 0.03% Triton X-100, 2.5 mM TCEP, 50 mM Imidazole). Finally fusion proteins were eluted with 250 μL of elution buffer (50 mM Sodium Phosphate pH7.7, 300 mM Sodium Chloride, 2.5 mM TCEP, 0.2% Tween and 250 mM Imidazole). The preparations were frozen at −20° C. in 50% glycerol, 50 mM Sodium Phosphate pH 7.7, 300 mM Sodium Chloride, 5 mM TCEP, 0.2% Tween and 125 mM Imidazole.

The following fusion proteins were analyzed by SDS-PAGE using 4-12% Bis-Tris polyacrylamide gels with SYPRO Orange fluorescent stain (Bio-Rad):
1. Nucleic acid binding protein fused to the N-terminus of truncated R2 enzyme (i.e., R2 enzyme minus the N-terminal DNA binding domain):
   Lane 1N is sso7d-ΔR2 enzyme (SEQ ID NO: 7)
   Lane 2N is Cren7-ΔR2 enzyme (SEQ ID NO: 11)
   Lane 3N is Cren7-Cren7-ΔR2 enzyme (SEQ ID NO: 13)
   Lane 4N is SSB-ΔR2 enzyme (SEQ ID NO: 15)
   Lane 5N is ΔR2 enzyme (SEQ ID NO: 2)
2. Nucleic acid binding protein fused to the C-terminus of truncated R2 enzyme
   Lane 1C is ΔR2 enzyme-Sso7d (SEQ ID NO: 19)
   Lane 2C is ΔR2 enzyme-SSB (SEQ ID NO: 35)
   Lane 3C is ΔR2 enzyme-Cren7 (SEQ ID NO: 23)
   Lane 4C is ΔR2 enzyme (SEQ ID NO: 2)

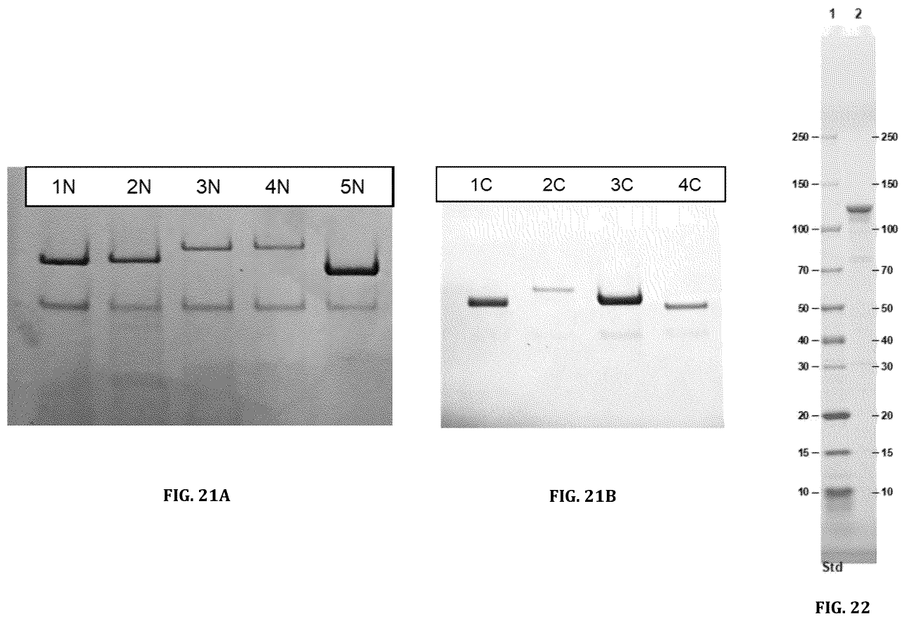

Images of the gels are shown in FIGS. 21A (N-terminal fusions) and 21B (C-terminal fusions). The results show that all of the fusion proteins were expressed.

### Example 2—qPCR with the Hybrid Reverse Transcriptases from Example 1

This example compares the qPCR performance of hybrid reverse transcriptases from Example 1 to truncated R2 enzyme that is not fused to a nucleic acid binding protein.

### Materials:

1. ΔR2 Enzyme (SEQ ID NO: 2) and the following hybrid reverse transcriptases (with a nucleic acid binding protein fused to the C-terminus of ΔR2 enzyme):
   a) ΔR2 enzyme-Sso7d (SEQ ID NO: 19)
   b) ΔR2 enzyme-Cren7 (SEQ ID NO: 23)
   c) ΔR2 enzyme-SSB (SEQ ID NO: 35)
2. 10× Fragmentation Buffer: 700 mM Tris-HCl, 100 mM MgCl₂, 50 mM DTT, pH 7.6
3. 100×dNTP: 90 mM dNTP in water
4. 5× Total RNA Buffer:

TABLE 9

| | Volume |
|---|---|
| 5× Total RNA Buffer | (μL) |
| H2O | 885 |
| 1000 mM Tris-HCl pH7.5 | 10 |
| 5000 mM NaCl2 | 60 |
| 1000 mM MgCl2 | 5 |
| 10% Tween | 25 |
| 1000 mM DTT | 15 |

5. nB Mix:

TABLE 10

| | Final | Volume |
|---|---|---|
| nB Mix | Concentration | (μL) |
| Water | | 212.3 |
| 5× Total RNA Buffer | 1× | 150 |
| 100 μM Primer P591 | 0.2 μM | 1.5 |
| 100× dNTPs | 1× | 7.5 |
| 100 μM P423 Acceptor Oligo | 0.5 μM | 3.75 |

6. nC Mix: 56.25 μL ΔR2 enzyme or Hybrid Reverse Transcriptase and 18.75 μL water.
7. cDNA elution buffer: 10 mM Tris, 1 mM EDTA pH 7.5 or water

8. RNA template External RNA Controls
Consortium (ERCC) 00074:
TGGACATTAATTAGGGCTGAAAGCCCTAACTTAATGGACGGGAG
GTATCCCAATAGGAGGTTTCCTCCTATGGTTTTCAAAACAATCA
CCATCATGCTATTAATGATATTAAAATCCCAACTATACCAAAGA
ATATCCCAATTATCCATAAAACTGTAACTAAGTGAGGCTCTCTC
ATTGGTTTATACTTCAATATAAGCCTTGGTAGGGATAGATAGCC
ACCTATATAGTATAGCTTCCCATCTTCTTTGAGAGTTGTTGGTT
TATGCTCATCCCTACTCATAACCCCAGCACTTAGATATTTTAAA
GAGGCATCTATCACATAAGGCATCATTATAACTAAAAATGGGAT
ATATTCCTTATAAACTACTGCTAAGACAGCTAAGAAAGCTCCAA
TTGGTAGAGTTCCAACATCTCCTGGAAAAACCTTTGCTGGATAT
TTGTTAAATATCAATAGCCCTAAATAGGATGCAGAGAATATCAA
AGCGGAAAAAATCCAAAAAAAAAAAAAAAAAAAAAAAA
9. RNA template ERCC 00002:
TCCAGATTACTTCCATTTCCGCCCAAGCTGCTCACAGTATACGG
GCGTCGGCATCCAGACCGTCGGCTGATCGTGGTTTTACTAGGCT
AGACTAGCGTACGAGCACTATGGTCAGTAATTCCTGGAGGAATA
GGTACCAAGAAAAAAACGAACCTTTGGGTTCCAGAGCTGTACGG
TCGCACTGAACTCGGATAGGTCTCAGAAAAACGAAATATAGGCT
TACGGTAGGTCCGAATGGCACAAAGCTTGTTCCGTTAGCTGGCA
TAAGATTCCATGCCTAGATGTGATACACGTTTCTGGAAACTGCC
TCGTCATGCGACTGTTCCCCGGGGTCAGGGCCGCTGGTATTTGC
TGTAAAGAGGGGCGTTGAGTCCGTCCGACTTCACTGCCCCCTTT
CAGCCTTTTGGGTCCTGTATCCCAATTCTCAGAGGTCCCGCCGT
ACGCTGAGGACCACCTGAAACGGGCATCGTCGCTCTTCGTTGTT
CGTCGACTTCTAGTGTGGAGACGAATTGCCAGAATTATTAACTG
CGCAGTTAGGGCAGCGTCTGAGGAAGTTTGCTGCGGTTTCGCCT
TGACCGCGGGAAGGAGACATAACGATAGCGACTCTGTCTCAGGG
GATCTGCATATGTTTGCAGCATACTTTAGGTGGGCCTTGGCTTC
CTTCCGCAGTCAAAACCGCGCAATTATCCCCGTCCTGATTTACT
GGACTCGCAACGTGGGTCCATCAGTTGTCCGTATACCAAGACGT
CTAAGGGCGGTGTACACCCTTTTGAGCAATGATTGCACAACCTG
CGATCACCTTATACAGAATTATCAATCAAGCTCCCCGAGGAGCG
GACTTGTAAGGACCGCCGCTTTCGCTCGGGTCTGCGGGTTATAG
CTTTTCAGTCTCGACGGGCTAGCACACATCTGGTTGACTAGGCG
CATAGTCGCCATTCACAGATTTGCTCGGCAATCAGTACTGGTAG
GCGTTAGACCCCGTGACTCGTGGCTGAACGGCCGTACAACTCGA
CAGCCGGTGCTTGCGTTTTACCCTTAAAAAAAAAAAAAAAAAAA
AAAAA
10. Acceptor-adapter P423:
AA/iSp9/ACACTCTTTCCCTACACGACGCTCTTCCGATCT/
3ddC/
11. Primer-adapter P591:
/5Sp9/GTGACTGGAGTTCAGACGTGTGCTCTTCCGATCT
12. PCR primer P312:
ACACTCTTTCCCTACACGACGCT
13. PCR primer G9:
CTGAGACCTATCCGAGTTCAGTGC
14. PCR primer C2:
GTGGCTATCTATCCCTACCAAGGCTTATATTG
15. Rotor-Gene® Q Real-Time PCR cycler
(Qiagen)

### Method:

1. cDNA preparation: For each enzyme and template RNA tested, 4 μL of 1/50 diluted template RNA, 5ρL water, and 1 μL of 10×Fragmentation Buffer was added to the enzyme in a tube and the solution was mixed. Heat was used to fragment the template RNA by incubating the solutions at 94° C. for 4 minutes, followed by 4° C. for 2 minutes. Next, 12.5 μL of nB was added to each solution, followed by mixing. Then, 2.5 μL of nC was added to each solution, followed by mixing. Each solution was then incubated at 34° C. for 45 minutes and stored at 4° C. until use.
2. SPRI cleanup (Beckman Coulter) was performed on each solution per manufacturer's instructions. For each solution, cDNA was eluted in 12 μL elution buffer and 3 uL was transferred to a new PCR tubes
3. qPCR: 3 μL of the eluted cDNA from Step 2 was mixed with 7.5 μL Luna® Universal qPCR Master Mix (NEB) and 4.5 μL water. qPCR was conducted according to the NEB protocol for Luna® Universal qPCR Master Mix using the Rotor-Gene® Q Real-Time PCR cycler (Qiagen). For RNA template ERCC 00074, primers P312 and C2 were used. For RNA template ERC 00002, primers P312 and G9 were used.

Results: The qPCR results in Table 8 show a yield improvement with the ΔR2 enzyme-SSB fusion and the ΔR2-Cren7 fusion.

TABLE 11

| | C_{T} values | |
|---|---|---|
| | RNA Template | RNA Template |
| Enzyme | ERCC 00074 | ERCC 00002 |
| ΔR2 enzyme | 26 | 20 |
| ΔR2 enzyme-Sso7d | 26 | 20 |
| ΔR2 enzyme-Cren7 | 24 | 19 |
| ΔR2 enzyme-SSB | 22 | 16 |

### Example 3—Processivity of Hybrid Reverse Transcriptases from Examples 1, 6, and 8

For each hybrid reverse transcriptase from Examples 1, 6, and 8, qPCR is used to quantify the amount (or yield) of cDNA copies obtained with an External RNA Controls Consortium (ERCC) standard mix of 90 different RNA templates having known concentrations, lengths, and secondary structure (or GC content). Appropriate primers for the RNA templates are used. The ratio of amount of long to short transcripts and/or high to low secondary structure-containing transcripts is measured. Highly processive and strand displacing enzymes with high affinity will generate exact cDNA copies of the RNA templates with the original ratio of long to short RNA templates or high to low secondary structure. Enzymes that exhibit low processivity, strand displacement, and low affinity will predominantly copy short RNA templates having low amounts of secondary structure.

### Example 4—Expression and Purification of a Dimer of Truncated SSB Recombinantly Fused to the C-Terminus of ΔR2 Enzyme (SEQ ID NO: 47)

23.3 grams of cell biomass containing a dimer of truncated SSB fused to the C-terminus of ΔR2 enzyme was re-suspended in 233 mL (10 mL per gram biomass) lysis buffer (1× BugBuster, 100 mM sodium phosphate pH 7.7, 0.1% Tween 20, 2.5 mM Tris (2-carboxyethyl) phosphine (TCEP), 0.1 mg/mL lysozyme, 1.25 units/mL DNase. The suspension was incubated for 45 minutes at room temperature (RT) with gently mixing. The lysate was then diluted 1:1 with Immobilized Metal Affinity Chromatography (IMAC) buffer A (50 mM sodium phosphate pH 7.7, 1.5 M NaCl, 2.5 mM TCEP, 0.1% Tween 20, 0.03% Triton X100, 10 mM imidazole) and was incubated at RT for 30 minutes with gentle rocking. The diluted lysate was sonicated and was clarified by centrifugation at 12,000 g for 40 minutes at 8° C. The supernatant was allowed to bind by gentle stirring to 10 mL Ni Sepharose FF6 resin (GE Healthcare) equilibrated with IMAC buffer A for 2 hours at 4° C. The resin was collected by centrifugation and poured onto a XK16 column (GE Healthcare). To step elute the fusion protein, the resin in the XK16 column was washed with 5 column volumes (CV) of IMAC buffer B (50 mM sodium phosphate pH 7.7, 300 mM M NaCl, 2.5 mM TCEP, 0.1% Tween 20, 250 mM imidazole). Fractions were pooled based on SDS-PAGE (not shown) and Western blot using (anti-histidine; not shown). The pooled fractions were then diluted 4-fold in heparin buffer (50 mM sodium phosphate pH 7.7, 100 mM NaCl, 2.5 mM TCEP, 0.1% Tween 20) and was loaded onto a 5 mM HiTrap Heparin HP column (GE Healthcare) equilibrated in heparin buffer. The column was washed with 10 CV heparin buffer followed by 10 CV heparin buffer with 300 mM NaCl. The bound fusion protein was eluted with a 20 CV gradient to heparin buffer with 1200 mM NaCl. Fractions to pool were determined by SDS-PAGE (not shown). The pooled heparin fractions were dialyzed at 4° C. overnight versus 20 mM sodium phosphate pH 7.7, 300 mM NaCl, 5 mM TCEP, 0.3% Tween 20, 10% glycerol.

The resulting purified fusion protein was analyzed by SDS-PAGE using 4-12% Bis-Tris polyacrylamide gels with Coomassie stain (see FIG. 22 for an image of the gel). The results show that the dimer of truncated SSB fused to the C-terminus of ΔR2 enzyme was expressed.

### Example 5—Expression and Purification of Fusion Proteins Comprising SpyTag and SpyCatcher Binding Motifs

Expression vector pET-45b carrying the constructs shown in Table 12 are transformed into E. coli BL21 (DE3).

TABLE 12

| SEQ ID | | Linker | |
|---|---|---|---|
| NO: | N-terminal ORF | ORF | C-terminal ORF |
| 69-70 | ΔR2 enzyme | Linker | SpyTag-linker-His Tag and |
| | | | Spy Catcher-linker-His Tag |
| 71-72 | Sso7d | Linker | SpyTag-linker-His Tag and |
| | | | Spy Catcher-linker-His Tag |
| 73-74 | Cren7 | Linker | SpyTag-linker-His Tag and |
| | | | Spy Catcher-linker-His Tag |
| 75-76 | SSB | Linker | SpyTag-linker-His Tag and |
| | | | Spy Catcher-linker-His Tag |
| 77-78 | His Tag-linker-SpyTag | Linker | ΔR2 enzyme |
| | and His Tag-linker-Spy | | |
| | Catcher | | |
| 79-80 | His Tag-linker-SpyTag | Linker | Sso7d |
| | and His Tag-linker-Spy | | |
| | Catcher | | |
| 81-82 | His Tag-linker-SpyTag | Linker | Cren7 |
| | and His Tag-linker-Spy | | |
| | Catcher | | |
| 83-84 | His Tag-linker-SpyTag | Linker | SSB |
| | and His Tag-linker-Spy | | |
| | Catcher | | |

For expression of each construct, a pre-culture are setup in 2 mL LB with 100 μM Corbenicillin and re grown for about 8-12 hours at room temperature. After about 8-12 hours, 550 μL of the pre-cultures are transferred to 25 mL auto-induction expression media (Overnight Express TB from Novagen) including 0.08 mg/ml Corbenicillin and shaker-incubated at 280-290 RPM at room temperature for 48 hours. Cells are harvested by centrifugation at 8000×g for 10 min at 4-8° C. The biomass-pellets are frozen at −20° C. for a minimum of 1 hour.

The pellets are re-suspended in 0.8 mL lysis buffer (0.8 ml lysis buffer per 10% of the biomass) and were incubated for 45 minutes at room temperature. Lysis buffer composition: 1×BugBuster (Millipore), 100 mM Sodium Phosphate, 0.2% Tween, 2.5 mM TCEP, 6 μl Protease inhibitor mix (Roche), 0.1 mg/ml lysozyme, 0.5 ul DNaseI (2,000 units/ml, from NEB). After incubation, the lysates are mixed with equal volume (0.8 mL) of His-binding buffer (50 mM Sodium Phosphate pH 7.7, 1.5 M Sodium Chloride, 5 mM TCEP, 0.2% Tween, 0.03% Triton X-100, 10 mM Imidazole) and incubated at room temperature for 30 minutes. After incubation, the lysates are centrifuged at 13000×g for 40 minutes at 80° C. The pellets are each mixed with 250 μl of His-Affinity Gel (His-Spin Protein Miniprep by Zymo Research) per the manufacturer's protocol. After the binding step, for each pellet, the His-Affinity Gel is washed five times with Washing buffer (50 mM Sodium Phosphate pH7.7, 1.2M Sodium Chloride, 0.2% Tween, 0.03% Triton X-100, 2.5 mM TCEP, 50 mM Imidazole). Finally fusion proteins are eluted with 250 μL of elution buffer (50 mM Sodium Phosphate pH7.7, 300 mM Sodium Chloride, 2.5 mM TCEP, 0.2% Tween and 250 mM Imidazole). The preparations are frozen at −20° C. in 50% glycerol, 50 mM Sodium Phosphate pH 7.7, 300 mM Sodium Chloride, 5 mM TCEP, 0.2% Tween and 125 mM Imidazole.

The fusion proteins are analyzed by SDS-PAGE using 4-12% Bis-Tris polyacrylamide gels with SYPRO Orange fluorescent stain (Bio-Rad).

### Example 6—Ligation of ΔR2 Enzyme-SpyTag and Sso7d-SpyCatcher, Cren7-SpyCatcher, or SSB-SpyCatcher to Form Hybrid Reverse Transcriptases

ΔR2 enzyme-SpyTag is ligated to Sso7d-SpyCatcher, Cren7-SpyCatcher, or SSB-SpyCatcher by reacting equimolar amounts of ΔR2 enzyme-SpyTag with Sso7d-SpyCatcher, Cren7-SpyCatcher, or SSB-SpyCatcher. The ligation reaction is allowed to proceed for about 16 hours at room temperature. For analysis, SDS loading buffer is added to the reaction mixture and the mixture is heated for 5 minutes at 95° C. prior to SDS-PAGE on 4-20% polyacrylamide gels. An image of the gel is obtained and shows that ΔR2 enzyme-SpyTag ligates with Sso7d-SpyCatcher, Cren7-SpyCatcher, or SSB-SpyCatcher.

### Example 7—Expression and Purification of Fusion Proteins Comprising AviTag Acceptor Peptide

Expression vector pET-45b carrying the constructs shown in Table 13 are transformed into E. coli BL21 (DE3).

TABLE 13

| SEQ ID | | Linker | |
|---|---|---|---|
| NO: | N-terminal ORF | ORF | C-terminal ORF |
| 90 | His Tag-linker-ΔR2 enzyme | Linker | AviTag |
| 91 | His Tag-linker-Sso7d | Linker | AviTag |
| 92 | His Tag-linker-Cren7 | Linker | AviTag |
| 93 | His Tag-linker-SSB | Linker | AviTag |
| 94 | His Tag-linker-AviTag | Linker | ΔR2 enzyme |
| 95 | His Tag-linker-AviTag | Linker | Sso7d |
| 96 | His Tag-linker-AviTag | Linker | Cren7 |
| 97 | His Tag-linker-AviTag | Linker | SSB |

For expression of each construct, a pre-culture are setup in 2 mL LB with 100 μM Corbenicillin and re grown for about 8-12 hours at room temperature. After about 8-12 hours, 550 μL of the pre-cultures are transferred to 25 mL auto-induction expression media (Overnight Express TB from Novagen) including 0.08 mg/ml Corbenicillin and shaker-incubated at 280-290 RPM at room temperature for 48 hours. Cells are harvested by centrifugation at 8000×g for 10 min at 4-8° C. The biomass-pellets are frozen at −20° C. for a minimum of 1 hour.

The pellets are re-suspended in 0.8 mL lysis buffer (0.8 ml lysis buffer per 10% of the biomass) and were incubated for 45 minutes at room temperature. Lysis buffer composition: 1×BugBuster (Millipore), 100 mM Sodium Phosphate, 0.2% Tween, 2.5 mM TCEP, 6 μl Protease inhibitor mix (Roche), 0.1 mg/ml lysozyme, 0.5 ul DNaseI (2,000 units/ml, from NEB). After incubation, the lysates are mixed with equal volume (0.8 mL) of His-binding buffer (50 mM Sodium Phosphate pH 7.7, 1.5 M Sodium Chloride, 5 mM TCEP, 0.2% Tween, 0.03% Triton X-100, 10 mM Imidazole) and incubated at room temperature for 30 minutes. After incubation, the lysates are centrifuged at 13000×g for 40 minutes at 80° C. The pellets are each mixed with 250 μl of His-Affinity Gel (His-Spin Protein Miniprep by Zymo Research) per the manufacturer's protocol. After the binding step, for each pellet, the His-Affinity Gel is washed five times with Washing buffer (50 mM Sodium Phosphate pH7.7, 1.2M Sodium Chloride, 0.2% Tween, 0.03% Triton X-100, 2.5 mM TCEP, 50 mM Imidazole). Finally fusion proteins are eluted with 250 μL of elution buffer (50 mM Sodium Phosphate pH7.7, 300 mM Sodium Chloride, 2.5 mM TCEP, 0.2% Tween and 250 mM Imidazole). The preparations are frozen at −20° C. in 50% glycerol, 50 mM Sodium Phosphate pH 7.7, 300 mM Sodium Chloride, 5 mM TCEP, 0.2% Tween and 125 mM Imidazole.

The fusion proteins are analyzed by SDS-PAGE using 4-12% Bis-Tris polyacrylamide gels with SYPRO Orange fluorescent stain (Bio-Rad).

### Example 8—Preparation of Hybrid Reverse Transcriptases with Biotinylated Fusion Protein Non-Covalently Bound to Streptavidin

To biotinylate the AviTag portion of the fusion proteins from Example 7, each fusion protein is mixed with 0.1 mM biotin and 30 nM BirA enzyme in the presence of 1 mM ATP and 5 mM MgCl₂ and allowed to incubate at 37° C. for 2 hours. The levels of biotinylation are analyzed via western blot using Strepavidin-HRP and HRP conjugated secondary antibody.

To non-covalently bind the biotinylated fusion proteins to streptavidin, different combinations of biotinylated fusion proteins are mixed with streptavidin in an appropriate ratio and incubated at 4° C. for 1 hour. Table 14 lists the combinations of ΔR2 enzyme comprising biotinylated AviTag and nucleic acid binding protein comprising biotinylated AviTag that are mixed with streptavidin along with the molar ratios to be used (i.e., that takes into the tetrameric structure of streptavidin):

TABLE 14

| 1^{st} Fusion | | |
|---|---|---|
| Protein or | | |
| 1^{st} and 2^{nd} | | |
| fusion | 2^{nd} Fusion Protein or | |
| protein | 3^{rd} and 4^{th} fusion protein | Molar Ratio |
| ΔR2 | biotinylated AviTag-Sso7d, | 2 moles 1^{st} fusion: |
| enzyme- | biotinylated AviTag-Cren7, | 2 moles |
| biotinylated | or biotinylated AviTag-SSB | 2^{nd} fusion: 1 mole |
| AviTag | | streptavidin |
| biotinylated | Sso7d-biotinylated AviTag, | 2 moles 1^{st} fusion: |
| AviTag- | Cren7-biotinylated AviTag, | 2 moles |
| ΔR2 enzyme | or SSB-biotinylated AviTag | 2^{nd} fusion: 1 mole |
| | | streptavidin |
| ΔR2 | Sso7d-biotinylated AviTag + | 1 mole 1^{st} fusion: |
| enzyme- | biotinylated AviTag-Sso7d, | 1 moles |
| biotinylated | Cren7-biotinylated AviTag + | 2^{nd} fusion: 1 mole |
| AviTag + | biotinylated AviTag-Cren7, | 3^{rd} fusion: 1 moles |
| biotinylated | or SSB-biotinylated AviTag + | 4^{th} fusion: 1 mole |
| AviTag- | biotinylated AviTag-SSB | streptavidin |
| ΔR2 enzyme | | |
| ΔR2 | biotinylated AviTag-ΔR2 | 2 moles 1^{st} fusion: |
| enzyme- | enzyme | 2 moles |
| biotinylated | | 2^{nd} fusion: 1 mole |
| AviTag | | streptavidin |

For analysis, SDS loading buffer is added to the reaction mixture and the mixture is heated for 5 minutes at 95° C. prior to SDS-PAGE on 4-20% polyacrylamide gels under non-reducing conditions. An image of the gel is obtained and shows biotinylated fusion proteins non-covalently bind to streptavidin. After SDS-PAGE analysis, unreacted biotinylated fusion protein and streptavidin are removed from each reaction by size exclusion chromatography, resulting in purified hybrid reverse transcriptases.

All patents, patent applications, and other published reference materials cited in this specification are hereby incorporated herein by reference in their entirety.

What is claimed is:

1. A method of preparing a cDNA molecule comprising:
(a) contacting a template RNA molecule and free nucleotides with:
i. a primer that is not complementary to the template RNA molecule;
ii. an acceptor-adapter; and
iii. a hybrid reverse transcriptase comprising a non-retroviral retrotransposon joined to a single stranded deoxyribonucleic acid (ssDNA) binding protein or a fragment of the ssDNA binding protein that binds to nucleic acid; and
(b) allowing the hybrid reverse transcriptase to transcribe the template RNA molecule under conditions effective for producing a cDNA molecule complementary to the RNA molecule and, optionally, to the acceptor-adapter, wherein the hybrid reverse transcriptase jumps to a 3′-end of the acceptor-adapter upon reaching a 5′ end of the RNA molecule.

2. The method of claim 1, wherein the hybrid reverse transcriptase comprises SEQ ID NO: 14, SEQ ID NO: 15, SEQ ID NO: 16, SEQ ID NO: 17, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 47, SEQ ID NO: 98, SEQ ID NO: 99, or SEQ ID NO: 100, or a sequence at least 75% identical to SEQ ID NO: 14, SEQ ID NO: 15, SEQ ID NO: 16, SEQ ID NO: 17, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 47, SEQ ID NO: 98, SEQ ID NO: 99, or SEQ ID NO: 100.

3. The method of claim 2, wherein the hybrid reverse transcriptase comprises at least one improved property selected from the group consisting of higher processivity, longer shelf life, higher strand displacement, higher end-to-end template jumping, and higher affinity as compared to a non-retroviral retrotransposon, or a fragment of the non-retroviral retrotransposon, that is not joined to a nucleic acid binding protein.

4. The method of claim 3, wherein the improved property of the hybrid reverse transcriptase is higher affinity.

5. The method of claim 1, wherein the non-retroviral retrotransposon, or the fragment of the non-retroviral retrotransposon, is joined via a linker to the nucleic acid binding protein or the fragment of the nucleic acid binding protein.

6. The method of claim 5, wherein the linker is selected from the group consisting of VGTVGTGGGSGGASTAL, VGTVGTGGGSEAAAKGGASTAL, VGTGGGSEAAAKGGASTAL, VGTGGGSGGGEAAAKEAAAKSGGGS, VGTGGGSGGGEAAAKEAAAKSGGGSA, VGTGGGSGGGTGGGS, VGTGGGSGGGTGGGSA, (GGGS)n, (GGS)n, (GGGGS)n, and (EAAAK)n and n is 1, 2, 3, 4, or 5.

7. The method of claim 1, wherein the nucleic acid binding protein, or the fragment of the nucleic acid binding protein, is joined to the N-terminus or C-terminus of the non-retroviral retrotransposon or the fragment of the non-retroviral retrotransposon.

8. The method of claim 1, wherein the nucleic acid binding protein is two or more nucleic acid binding proteins or fragments of two or more nucleic acid binding proteins and the two or more nucleic acid binding proteins or fragments of two or more nucleic acid binding proteins are identical or non-identical.

9. The method of claim 8, wherein the two or more nucleic acid binding proteins or fragments of two or more nucleic acid binding proteins are in sequential or random order.

10. The method of claim 1, wherein the non-retroviral retrotransposon, or the fragment of the non-retroviral retrotransposon, is covalently joined to the nucleic acid binding protein or the fragment of the nucleic acid binding protein.

11. A method of preparing a cDNA molecule library comprising:
(a) fragmenting a template RNA molecule to produce RNA fragments;
(b) contacting the RNA fragments and free nucleotides, with:
i. a primer-adapter that is not complementary to the RNA fragments;
ii. an acceptor-adapter; and
iii. a hybrid reverse transcriptase comprising a non-retroviral retrotransposon joined to a single stranded deoxyribonucleic acid (ssDNA) binding protein or a fragment of the ssDNA binding protein that binds to nucleic acid; and
(c) allowing the hybrid reverse transcriptase to transcribe the RNA fragments under conditions effective to produce a cDNA molecule library, wherein the hybrid reverse transcriptase jumps to a 3′-end of the acceptor-adapter upon reaching a 5′ end of the RNA fragments.

12. The method of claim 11, wherein the hybrid reverse transcriptase comprises SEQ ID NO: 14, SEQ ID NO: 15, SEQ ID NO: 16, SEQ ID NO: 17, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 47, SEQ ID NO: 98, SEQ ID NO: 99, or SEQ ID NO: 100, or a sequence at least 75% identical to SEQ ID NO: 14, SEQ ID NO: 15, SEQ ID NO: 16, SEQ ID NO: 17, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 47, SEQ ID NO: 98, SEQ ID NO: 99, or SEQ ID NO: 100.

13. The method of claim 12, wherein the hybrid reverse transcriptase comprises at least one improved property selected from the group consisting of higher processivity, longer shelf life, higher strand displacement, higher end-to-end template jumping, and higher affinity as compared to a non-retroviral retrotransposon, or a fragment of the non-retroviral retrotransposon, that is not joined to a nucleic acid binding protein.

14. The method of claim 13, wherein the improved property of the hybrid reverse transcriptase is higher affinity.

15. The method of claim 11, wherein the non-retroviral retrotransposon, or the fragment of the non-retroviral retrotransposon, is joined via a linker to the nucleic acid binding protein or the fragment of the nucleic acid binding protein.

16. The method of claim 15, wherein the linker is selected from the group consisting of VGTVGTGGGSGGASTAL, VGTVGTGGGSEAAAKGGASTAL, VGTGGGSEAAAKGGASTAL, VGTGGGSGGGEAAAKEAAAKSGGGS, VGTGGGSGGGEAAAKEAAAKSGGGSA, VGTGGGSGGGTGGGS, VGTGGGSGGGTGGGSA, (GGGS)n, (GGS)n, (GGGGS)n, and (EAAAK)n and n is 1, 2, 3, 4, or 5.

17. The method of claim 11, wherein the nucleic acid binding protein, or the fragment of the nucleic acid binding protein, is joined to the N-terminus or C-terminus of the non-retroviral retrotransposon or the fragment of the non-retroviral retrotransposon.

18. The method of claim 11, wherein the nucleic acid binding protein is two or more nucleic acid binding proteins or fragments of two or more nucleic acid binding proteins and the two or more nucleic acid binding proteins or fragments of two or more nucleic acid binding proteins are identical or non-identical.

19. The method of claim 18, wherein the two or more nucleic acid binding proteins or fragments of two or more nucleic acid binding proteins are in sequential or random order.

20. The method of claim 11, wherein the non-retroviral retrotransposon, or the fragment of the non-retroviral retrotransposon, is covalently joined to the nucleic acid binding protein or the fragment of the nucleic acid binding protein.


SEQUENCE LISTING (124 sequences)

SEQ ID NO: 1 - protein, 1114 aa, Bombyx mori
  REGION 1..1114: note = misc_feature - wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 2 - protein, 829 aa, synthetic construct
  REGION 1..829: note = deltaR2 enzyme or R2 enzyme minus N-terminal DNA binding domain
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 3 - protein, 63 aa, synthetic construct
  REGION 1..63: note = Sso7d
ATVKFKYKGEEKEVDISKIKKVWRVGKMISFTYDEGGGKTGRGAVSEKDAPKELLQMLEKQKK

SEQ ID NO: 4 - protein, 59 aa, synthetic construct
  REGION 1..59: note = Cren7
SSGKKPVKVKTPAGKEAELVPEKVWALAPKGRKGVKIGLFKDPETGKYFRHKLPDDYPI

SEQ ID NO: 5 - protein, 148 aa, synthetic construct
  REGION 1..148: note = Sulfolobus SSB
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 6 - protein, 1194 aa, synthetic construct
  REGION 1..1194: note = Sso7d joined via a linker to the N-terminus of wild type R2 enzyme
ATVKFKYKGEEKEVDISKIKKVWRVGKMISFTYDEGGGKTGRGAVSEKDAPKELLQMLEKQKKVGTVGTGGGSGGASTALMMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 7 - protein, 934 aa, synthetic construct
  REGION 1..934: note = His Tag joined via a linker to Sso7d joined via a linker to the N-terminus of deltaR2 enzyme
MAHHHHHHVGTVGTGGGSGGASTALATVKFKYKGEEKEVDISKIKKVWRVGKMISFTYDEGGGKTGRGAVSEKDAPKELLQMLEKQKKVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 8 - protein, 1287 aa, synthetic construct
  REGION 1..1287: note = Sso7d-Sso7d joined via a linker to the N-terminus of wild type R2 enzyme
ATVKFKYKGEEKEVDISKIKKVWRVGKMISFTYDEGGGKTGRGAVSEKDAPKELLQMLEKQKKVGTGGGSGGGEAAAKEAAAKSGGGSATVKFKYKGEEKEVDISKIKKVWRVGKMISFTYDEGGGKTGRGAVSEKDAPKELLQMLEKQKKVGTVGTGGGSEAAAKGGASTALMMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 9 - protein, 1002 aa, synthetic construct
  REGION 1..1002: note = Sso7d-Sso7d joined via a linker to the N-terminus of deltaR2 enzyme
ATVKFKYKGEEKEVDISKIKKVWRVGKMISFTYDEGGGKTGRGAVSEKDAPKELLQMLEKQKKVGTGGGSGGGEAAAKEAAAKSGGGSATVKFKYKGEEKEVDISKIKKVWRVGKMISFTYDEGGGKTGRGAVSEKDAPKELLQMLEKQKKVGTVGTGGGSEAAAKGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 10 - protein, 1195 aa, synthetic construct
  REGION 1..1195: note = Cren7 joined via a linker to the N-terminus of wild type R2 enzyme
SSGKKPVKVKTPAGKEAELVPEKVWALAPKGRKGVKIGLFKDPETGKYFRHKLPDDYPIVGTVGTGGGSEAAAKGGASTALMMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 11 - protein, 935 aa, synthetic construct
  REGION 1..935: note = His Tag joined via a linker to Cren7 joined via a linker to the N-terminus of deltaR2 enzyme
MAHHHHHHVGTVGTGGGSGGASTALSSGKKPVKVKTPAGKEAELVPEKVWALAPKGRKGVKIGLFKDPETGKYFRHKLPDDYPIVGTVGTGGGSEAAAKGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 12 - protein, 1280 aa, synthetic construct
  REGION 1..1280: note = Cren7-Cren7 joined via a linker to the N-terminus of wild type R2 enzyme
SSGKKPVKVKTPAGKEAELVPEKVWALAPKGRKGVKIGLFKDPETGKYFRHKLPDDYPIVGTGGGSGGGEAAAKEAAAKSGGGSASSGKKPVKVKTPAGKEAELVPEKVWALAPKGRKGVKIGLFKDPETGKYFRHKLPDDYPIVGTVGTGGGSEAAAKGGASTALMMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 13 - protein, 1020 aa, synthetic construct
  REGION 1..1020: note = His Tag joined via a linker to Cren7-Cren7 joined via a linker to the N-terminus of deltaR2 enzyme
MAHHHHHHVGTVGTGGGSGGASTALSSGKKPVKVKTPAGKEAELVPEKVWALAPKGRKGVKIGLFKDPETGKYFRHKLPDDYPIVGTGGGSGGGEAAAKEAAAKSGGGSASSGKKPVKVKTPAGKEAELVPEKVWALAPKGRKGVKIGLFKDPETGKYFRHKLPDDYPIVGTVGTGGGSEAAAKGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 14 - protein, 1283 aa, synthetic construct
  REGION 1..1283: note = SSB joined via a linker to the N-terminus of wild type R2 enzyme
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEGTVGTGGGSEAAAKGGASTALMMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 15 - protein, 1023 aa, synthetic construct
  REGION 1..1023: note = His Tag joined via a linker to SSB joined via a linker to the N-terminus of deltaR2 enzyme
MAHHHHHHVGTVGTGGGSGGASTALMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEGTVGTGGGSEAAAKGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 16 - protein, 1456 aa, synthetic construct
  REGION 1..1456: note = SSB-SSB joined via a linker to the N-terminus of wild type R2 enzyme
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGEAAAKEAAAKSGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEGTVGTGGGSEAAAKGGASTALMMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 17 - protein, 1171 aa, synthetic construct
  REGION 1..1171: note = SSB-SSB joined via a linker to the N-terminus of deltaR2 enzyme
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGEAAAKEAAAKSGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEGTVGTGGGSEAAAKGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 18 - protein, 1192 aa, synthetic construct
  REGION 1..1192: note = Sso7d joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSATVKFKYKGEEKEVDISKIKKVWRVGKMISFTYDEGGGKTGRGAVSEKDAPKELLQMLEKQKK

SEQ ID NO: 19 - protein, 932 aa, synthetic construct
  REGION 1..932: note = His Tag joined via a linker to Sso7d joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSATVKFKYKGEEKEVDISKIKKVWRVGKMISFTYDEGGGKTGRGAVSEKDAPKELLQMLEKQKK

SEQ ID NO: 20 - protein, 1270 aa, synthetic construct
  REGION 1..1270: note = Sso7d-Sso7d joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSATVKFKYKGEEKEVDISKIKKVWRVGKMISFTYDEGGGKTGRGAVSEKDAPKELLQMLEKQKKVGTGGGSGGGTGGGSATVKFKYKGEEKEVDISKIKKVWRVGKMISFTYDEGGGKTGRGAVSEKDAPKELLQMLEKQKK

SEQ ID NO: 21 - protein, 985 aa, synthetic construct
  REGION 1..985: note = Sso7d-Sso7d joined via a linker to the C-terminus of deltaR2 enzyme
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSATVKFKYKGEEKEVDISKIKKVWRVGKMISFTYDEGGGKTGRGAVSEKDAPKELLQMLEKQKKVGTGGGSGGGTGGGSATVKFKYKGEEKEVDISKIKKVWRVGKMISFTYDEGGGKTGRGAVSEKDAPKELLQMLEKQKK

SEQ ID NO: 22 - protein, 1189 aa, synthetic construct
  REGION 1..1189: note = Cren7 joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSASSGKKPVKVKTPAGKEAELVPEKVWALAPKGRKGVKIGLFKDPETGKYFRHKLPDDYPI

SEQ ID NO: 23 - protein, 929 aa, synthetic construct
  REGION 1..929: note = His Tag joined via a linker to Cren7 joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSASSGKKPVKVKTPAGKEAELVPEKVWALAPKGRKGVKIGLFKDPETGKYFRHKLPDDYPI

SEQ ID NO: 24 - protein, 1264 aa, synthetic construct
  REGION 1..1264: note = Cren7-Cren7 joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSASSGKKPVKVKTPAGKEAELVPEKVWALAPKGRKGVKIGLFKDPETGKYFRHKLPDDYPIVGTGGGSGGGTGGGSASSGKKPVKVKTPAGKEAELVPEKVWALAPKGRKGVKIGLFKDPETGKYFRHKLPDDYPI

SEQ ID NO: 25 - protein, 979 aa, synthetic construct
  REGION 1..979: note = Cren7-Cren7 joined via a linker to the C-terminus of deltaR2 enzyme
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSASSGKKPVKVKTPAGKEAELVPEKVWALAPKGRKGVKIGLFKDPETGKYFRHKLPDDYPIVGTGGGSGGGTGGGSASSGKKPVKVKTPAGKEAELVPEKVWALAPKGRKGVKIGLFKDPETGKYFRHKLPDDYPI

SEQ ID NO: 26 - protein, 1339 aa, synthetic construct
  REGION 1..1339: note = Cren7-Cren7-Cren7 joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSASSGKKPVKVKTPAGKEAELVPEKVWALAPKGRKGVKIGLFKDPETGKYFRHKLPDDYPIVGTGGGSGGGTGGGSASSGKKPVKVKTPAGKEAELVPEKVWALAPKGRKGVKIGLFKDPETGKYFRHKLPDDYPIVGTGGGSGGGTGGGSASSGKKPVKVKTPAGKEAELVPEKVWALAPKGRKGVKIGLFKDPETGKYFRHKLPDDYPI

SEQ ID NO: 27 - protein, 1079 aa, synthetic construct
  REGION 1..1079: note = His Tag joined via a linker to Cren7-Cren7-Cren7 joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSASSGKKPVKVKTPAGKEAELVPEKVWALAPKGRKGVKIGLFKDPETGKYFRHKLPDDYPIVGTGGGSGGGTGGGSASSGKKPVKVKTPAGKEAELVPEKVWALAPKGRKGVKIGLFKDPETGKYFRHKLPDDYPIVGTGGGSGGGTGGGSASSGKKPVKVKTPAGKEAELVPEKVWALAPKGRKGVKIGLFKDPETGKYFRHKLPDDYPI

SEQ ID NO: 28 - protein, 1267 aa, synthetic construct
  REGION 1..1267: note = Sso7d-Cren7 joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSATVKFKYKGEEKEVDISKIKKVWRVGKMISFTYDEGGGKTGRGAVSEKDAPKELLQMLEKQKKVGTGGGSGGGTGGGSASSGKKPVKVKTPAGKEAELVPEKVWALAPKGRKGVKIGLFKDPETGKYFRHKLPDDYPI

SEQ ID NO: 29 - protein, 1007 aa, synthetic construct
  REGION 1..1007: note = His Tag joined via a linker to Sso7d-Cren7 joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSATVKFKYKGEEKEVDISKIKKVWRVGKMISFTYDEGGGKTGRGAVSEKDAPKELLQMLEKQKKVGTGGGSGGGTGGGSASSGKKPVKVKTPAGKEAELVPEKVWALAPKGRKGVKIGLFKDPETGKYFRHKLPDDYPI

SEQ ID NO: 30 - protein, 1352 aa, synthetic construct
  REGION 1..1352: note = Sso7d-Cren7-Cren7 joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSATVKFKYKGEEKEVDISKIKKVWRVGKMISFTYDEGGGKTGRGAVSEKDAPKELLQMLEKQKKVGTGGGSGGGTGGGSASSGKKPVKVKTPAGKEAELVPEKVWALAPKGRKGVKIGLFKDPETGKYFRHKLPDDYPIVGTGGGSGGGEAAAKEAAAKSGGGSASSGKKPVKVKTPAGKEAELVPEKVWALAPKGRKGVKIGLFKDPETGKYFRHKLPDDYPI

SEQ ID NO: 31 - protein, 1092 aa, synthetic construct
  REGION 1..1092: note = His Tag joined via a linker to Sso7d-Cren7-Cren7 joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSATVKFKYKGEEKEVDISKIKKVWRVGKMISFTYDEGGGKTGRGAVSEKDAPKELLQMLEKQKKVGTGGGSGGGTGGGSASSGKKPVKVKTPAGKEAELVPEKVWALAPKGRKGVKIGLFKDPETGKYFRHKLPDDYPIVGTGGGSGGGEAAAKEAAAKSGGGSASSGKKPVKVKTPAGKEAELVPEKVWALAPKGRKGVKIGLFKDPETGKYFRHKLPDDYPI

SEQ ID NO: 32 - protein, 1267 aa, synthetic construct
  REGION 1..1267: note = Cren7-Sso7d joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSASSGKKPVKVKTPAGKEAELVPEKVWALAPKGRKGVKIGLFKDPETGKYFRHKLPDDYPIVGTGGGSGGGTGGGSATVKFKYKGEEKEVDISKIKKVWRVGKMISFTYDEGGGKTGRGAVSEKDAPKELLQMLEKQKK

SEQ ID NO: 33 - protein, 982 aa, synthetic construct
  REGION 1..982: note = Cren7-Sso7d joined via a linker to the C-terminus of deltaR2 enzyme
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSASSGKKPVKVKTPAGKEAELVPEKVWALAPKGRKGVKIGLFKDPETGKYFRHKLPDDYPIVGTGGGSGGGTGGGSATVKFKYKGEEKEVDISKIKKVWRVGKMISFTYDEGGGKTGRGAVSEKDAPKELLQMLEKQKK

SEQ ID NO: 34 - protein, 1277 aa, synthetic construct
  REGION 1..1277: note = SSB joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 35 - protein, 1017 aa, synthetic construct
  REGION 1..1017: note = His Tag joined via a linker to SSB joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 36 - protein, 1440 aa, synthetic construct
  REGION 1..1440: note = SSB-SSB joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 37 - protein, 1180 aa, synthetic construct
  REGION 1..1180: note = His Tag joined via a linker to SSB-SSB joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 38 - protein, 1603 aa, synthetic construct
  REGION 1..1603: note = SSB-SSB-SSB joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 39 - protein, 1318 aa, synthetic construct
  REGION 1..1318: note = SSB-SSB-SSB joined via a linker to the C-terminus of deltaR2 enzyme
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 40 - protein, 1352 aa, synthetic construct
  REGION 1..1352: note = SSB-Cren7 joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSASSGKKPVKVKTPAGKEAELVPEKVWALAPKGRKGVKIGLFKDPETGKYFRHKLPDDYPI

SEQ ID NO: 41 - protein, 1092 aa, synthetic construct
  REGION 1..1092: note = His Tag joined via a linker to SSB-Cren7 joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSASSGKKPVKVKTPAGKEAELVPEKVWALAPKGRKGVKIGLFKDPETGKYFRHKLPDDYPI

SEQ ID NO: 42 - protein, 1426 aa, synthetic construct
  REGION 1..1426: note = SSB-Cren7-Cren7 joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSASSGKKPVKVKTPAGKEAELVPEKVWALAPKGRKGVKIGLFKDPETGKYFRHKLPDDYPIVGTGGGSGGGTGGGSASSGKKPVKVKTPAGKEAELVPEKVWALAPKGRKGVKIGLFKDPETGKYFRHKLPDDYP

SEQ ID NO: 43 - protein, 1141 aa, synthetic construct
  REGION 1..1141: note = SSB-Cren7-Cren7 joined via a linker to the C-terminus of deltaR2 enzyme
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEVGTGGGSGGGTGGGSASSGKKPVKVKTPAGKEAELVPEKVWALAPKGRKGVKIGLFKDPETGKYFRHKLPDDYPIVGTGGGSGGGTGGGSASSGKKPVKVKTPAGKEAELVPEKVWALAPKGRKGVKIGLFKDPETGKYFRHKLPDDYP

SEQ ID NO: 44 - protein, 2254 aa, synthetic construct
  REGION 1..2254: note = dimer of WT R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGEAAAKEAAAKSGGGSAMMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 45 - protein, 1684 aa, synthetic construct
  REGION 1..1684: note = dimer of deltaR2 enzyme
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGEAAAKEAAAKSGGGSAKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 46 - protein, 117 aa, synthetic construct
  REGION 1..117: note = C-terminal truncated SSB
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTARRR

SEQ ID NO: 47 - protein, 1121 aa, synthetic construct
  REGION 1..1121: note = His Tag joined via a linker to a dimer of truncated SSB joined via a linker to the C-terminus of deltaR2 enzyme
MAHHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTARRRGGGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTARRR

SEQ ID NO: 48 - protein, 13 aa, synthetic construct
  REGION 1..13: note = Spytag
AHIVMVDAYKPTK

SEQ ID NO: 49 - protein, 14 aa, synthetic construct
  REGION 1..14: note = SpyTag002
VPTIVMVDAYKRYK

SEQ ID NO: 50 - protein, 16 aa, synthetic construct
  REGION 1..16: note = Isopeptag
TDKDMTITFTNKKDAE

SEQ ID NO: 51 - protein, 115 aa, synthetic construct
  REGION 1..115: note = SpyCatcher
GAMVDTLSGLSSEQGQSGDMTIEEDSATHIKFSKRDEDGKELAGATMELRSSGKTISTWISDGQVKDFYLYPGKYTFVETAAPDGYEVATAITFTVNEQGQVTVNGKATKGDAHI

SEQ ID NO: 52 - protein, 119 aa, synthetic construct
  REGION 1..119: note = SpyCatcher002
AMVTTLSGLSGEQGPSGDMTTEEDSATHIKFSKRDEDGRELAGATMELRDSSGKTISTWSDGHVKDFYLYPGKYTFVETAAPDGYEVATAITFTVNEQGQVTVNGEATKGDAHTGSSGS

SEQ ID NO: 53 - protein, 83 aa, synthetic construct
  REGION 1..83: note = SpyCatcher short
GDSATHIKFSKRDEDGKELAGATMELRDSSGKTISTWISDGQVKDFYLYPKYTFVETAAPDGYEVATAITFTVNEQGQVTVNG

SEQ ID NO: 54 - protein, 282 aa, synthetic construct
  REGION 1..282: note = Split Spy0128
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 55 - protein, 10 aa, synthetic construct
  REGION 1..10: note = K-Tag
ATHIKFSKRD

SEQ ID NO: 56 - protein, 12 aa, synthetic construct
  REGION 1..12: note = SnoopTag
KLGDIEFIKVNK

SEQ ID NO: 57 - protein, 13 aa, synthetic construct
  REGION 1..13: note = SdyTag
DPIVMIDNDKPIT

SEQ ID NO: 58 - protein, 12 aa, synthetic construct
  REGION 1..12: note = SnoopTagJr
KLGSIEFIKVNK

SEQ ID NO: 59 - protein, 112 aa, synthetic construct
  REGION 1..112: note = SnoopCatcher
KPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 60 - protein, 104 aa, synthetic construct
  REGION 1..104: note = SdyCatcherDANG short
GRGSSGLSGETGQSGNTTIEEDSTTHVKFSKRDANGKELAGAMIELRNLSGQTIQSWISDGTVKVFYLMPGTYQFVETAAPEGYELAAPITFTIDEKGQIWVDS

SEQ ID NO: 61 - protein, 23 aa, synthetic construct
  REGION 1..23: note = DogTag
DIPATYEFTNGKHYITNEPIPPK

SEQ ID NO: 62 - protein, 6 aa, synthetic construct
  REGION 1..6: note = sortase A recognition domain
LPTGAA

SEQ ID NO: 63 - protein, 6 aa, synthetic construct
  REGION 1..6: note = sortase A recognition domain
LPTGGG

SEQ ID NO: 64 - protein, 6 aa, synthetic construct
  REGION 1..6: note = sortase A recognition domain
LPKTGG

SEQ ID NO: 65 - protein, 5 aa, synthetic construct
  REGION 1..5: note = sortase A recognition domain
LPETG

SEQ ID NO: 66 - protein, 5 aa, synthetic construct
  REGION 1..5: note = sortase A recognition domain
  SITE 3: note = MISC_FEATURE - any amino acid
LPXTG

SEQ ID NO: 67 - protein, 6 aa, synthetic construct
  REGION 1..6: note = sortase A recognition domain
  SITE 3: note = MISC_FEATURE - any amino acid
  PEPTIDE 6: note = peptide comprising any amino acids of length 1, 2, 3, 4, 5, 6, 7, 8, 9, or 10 amino acids
LPXTGX

SEQ ID NO: 68 - skipped

SEQ ID NO: 69 - protein, 882 aa, synthetic construct
  REGION 1..882: note = deltaR2 enzyme-linker-SpyTag-linker-His Tag
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSAHIVMVDAYKPTKKGVGTVGTGGGSGGASTALHHHHHH

SEQ ID NO: 70 - protein, 996 aa, synthetic construct
  REGION 1..996: note = deltaR2 enzyme-linker-SpyCatcher-linker-His Tag
KTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIVGTVGTGGGSGGASTALHHHHHH

SEQ ID NO: 71 - protein, 116 aa, synthetic construct
  REGION 1..116: note = Sso7d-linker-SpyTag-linker-His Tag
ATVKFKYKGEEKEVDISKIKKVWRVGKMISFTYDEGGGKTGRGAVSEKDAPKELLQMLEKQKKVGTGGGSGGGTGGGSAHIVMVDAYKPTKKGVGTVGTGGGSGGASTALHHHHHH

SEQ ID NO: 72 - protein, 230 aa, synthetic construct
  REGION 1..230: note = Sso7d-linker-SpyCatcher-linker-His Tag
ATVKFKYKGEEKEVDISKIKKVWRVGKMISFTYDEGGGKTGRGAVSEKDAPKELLQMLEKQKKVGTGGGSGGGTGGGSDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIVGTVGTGGGSGGASTALHHHHHH

SEQ ID NO: 73 - protein, 114 aa, synthetic construct
  REGION 1..114: note = Cren7-linker-SpyTag-linker-His Tag
SSGKKPVKVKTPAGKEAELVPEKVWALAPKGRKGVKIGLFKDPETGKYFRHKLPDDYPIVGTVGTGGGSEAAAKGGAHIVMVDAYKPTKKGVGTVGTGGGSGGASTALHHHHHH

SEQ ID NO: 74 - protein, 226 aa, synthetic construct
  REGION 1..226: note = Cren7-linker-SpyCatcher-linker-His Tag
SSGKKPVKVKTPAGKEAELVPEKVWALAPKGRKGVKIGLFKDPETGKYFRHKLPDDYPIVGTGGGSGGGTGGGSDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIVGTVGTGGGSGGASTALHHHHHH

SEQ ID NO: 75 - protein, 202 aa, synthetic construct
  REGION 1..202: note = SSB-linker-SpyTag-linker-His Tag
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEGTVGTGGGSEAAAKGGAHIVMVDAYKPTKKGVGTVGTGGGSGGASTALHHHHHH

SEQ ID NO: 76 - protein, 321 aa, synthetic construct
  REGION 1..321: note = SSB-linker-SpyCatcher-linker-His Tag
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEGTVGTGGGSEAAAKGGASTALDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIVGTVGTGGGSGGASTALHHHHHH

SEQ ID NO: 77 - protein, 884 aa, synthetic construct
  REGION 1..884: note = His Tag-linker-SpyTag-linker-deltaR2 enzyme
HHHHHHVGTVGTGGGSGGASTALAHIVMVDAYKPTKKGVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 78 - protein, 998 aa, synthetic construct
  REGION 1..998: note = His Tag-linker-SpyCatcher-linker-deltaR2 enzyme
HHHHHHVGTVGTGGGSGGASTALDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 79 - protein, 118 aa, synthetic construct
  REGION 1..118: note = His Tag-linker-SpyTag-linker-Sso7d
HHHHHHVGTVGTGGGSGGASTALAHIVMVDAYKPTKKGVGTVGTGGGSGGASTALATVKFKYKGEEKEVDISKIKKVWRVGKMISFTYDEGGGKTGRGAVSEKDAPKELLQMLEKQKK

SEQ ID NO: 80 - protein, 232 aa, synthetic construct
  REGION 1..232: note = His Tag-linker-SpyCatcher-linker-Sso7d
HHHHHHVGTVGTGGGSGGASTALDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIVGTVGTGGGSGGASTALATVKFKYKGEEKEVDISKIKKVWRVGKMISFTYDEGGGKTGRGAVSEKDAPKELLQMLEKQKK

SEQ ID NO: 81 - protein, 114 aa, synthetic construct
  REGION 1..114: note = His Tag-linker-SpyTag-linker-Cren7
HHHHHHVGTVGTGGGSGGASTALAHIVMVDAYKPTKKGVGTVGTGGGSGGASTALSSGKKPVKVKTPAGKEAELVPEKVWALAPKGRKGVKIGLFKDPETGKYFRHKLPDDYPI

SEQ ID NO: 82 - protein, 228 aa, synthetic construct
  REGION 1..228: note = His Tag-linker-SpyCatcher-linker-Cren7
HHHHHHVGTVGTGGGSGGASTALDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIVGTVGTGGGSGGASTALSSGKKPVKVKTPAGKEAELVPEKVWALAPKGRKGVKIGLFKDPETGKYFRHKLPDDYPI

SEQ ID NO: 83 - protein, 203 aa, synthetic construct
  REGION 1..203: note = His Tag-linker-SpyTag-linker-SSB
HHHHHHVGTVGTGGGSGGASTALAHIVMVDAYKPTKKGVGTVGTGGGSGGASTALMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 84 - protein, 315 aa, synthetic construct
  REGION 1..315: note = His Tag-linker-SpyCatcher-linker-SSB
HHHHHHVGTVGTGGGSGGASTALDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 85 - protein, 15 aa, synthetic construct
  REGION 1..15: note = AviTag
GLNDIFEAQKIEWHE

SEQ ID NO: 86 - protein, 15 aa, synthetic construct
  REGION 1..15: note = BioTag
ALNDIFEAQKIEWHA

SEQ ID NO: 87 - protein, 23 aa, synthetic construct
  REGION 1..23: note = Biotin ligase recognition peptide
MAGGLNDIFEAQKIEWHEDTGGS

SEQ ID NO: 88 - protein, 15 aa, synthetic construct
  REGION 1..15: note = BirA Substrate Peptide
LHHILDAQKMVWNHR

SEQ ID NO: 89 - protein, 13 aa, synthetic construct
  REGION 1..13: note = Synthetic
  SITE 2: note = MISC_FEATURE - any amino acid
  SITE 3: note = MISC_FEATURE - any amino acid other than L, V, I, W, F, or Y
LXXIFEAQKIEWR

SEQ ID NO: 90 - protein, 882 aa, synthetic construct
  REGION 1..882: note = His Tag-linker-deltaR2 enzyme-linker-AviTag
HHHHHHVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSGLNDIFEAQKIEWHE

SEQ ID NO: 91 - protein, 116 aa, synthetic construct
  REGION 1..116: note = His Tag-linker-Sso7d-linker-AviTag
HHHHHHVGTVGTGGGSGGASTALATVKFKYKGEEKEVDISKIKKVWRVGKMISFTYDEGGGKTGRGAVSEKDAPKELLQMLEKQKKVGTGGGSGGGTGGGSGLNDIFEAQKIEWHE

SEQ ID NO: 92 - protein, 114 aa, synthetic construct
  REGION 1..114: note = His Tag-linker-Cren7-linker-AviTag
HHHHHHVGTVGTGGGSGGASTALSSGKKPVKVKTPAGKEAELVPEKVWALAPKGRKGVKIGLFKDPETGKYFRHKLPDDYPIVGTVGTGGGSEAAAKGGGLNDIFEAQKIEWHE

SEQ ID NO: 93 - protein, 202 aa, synthetic construct
  REGION 1..202: note = His Tag-linker-SSB-linker-AviTag
HHHHHHVGTVGTGGGSGGASTALMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEEGTVGTGGGSEAAAKGGGLNDIFEAQKIEWHE

SEQ ID NO: 94 - protein, 884 aa, synthetic construct
  REGION 1..884: note = His Tag-linker-AviTag-linker-deltaR2 enzyme
HHHHHHVGTVGTGGGSGGASTALGLNDIFEAQKIEWHEVGTVGTGGGSGGASTALKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 95 - protein, 118 aa, synthetic construct
  REGION 1..118: note = His Tag-linker-AviTag-linker-Sso7d
HHHHHHVGTVGTGGGSGGASTALGLNDIFEAQKIEWHEVGTVGTGGGSGGASTALATVKFKYKGEEKEVDISKIKKVWRVGKMISFTYDEGGGKTGRGAVSEKDAPKELLQMLEKQKK

SEQ ID NO: 96 - protein, 114 aa, synthetic construct
  REGION 1..114: note = His Tag-linker-AviTag-linker-Cren7
HHHHHHVGTVGTGGGSGGASTALGLNDIFEAQKIEWHEVGTVGTGGGSGGASTALSSGKKPVKVKTPAGKEAELVPEKVWALAPKGRKGVKIGLFKDPETGKYFRHKLPDDYPI

SEQ ID NO: 97 - protein, 203 aa, synthetic construct
  REGION 1..203: note = His Tag-linker-AviTag-linker-SSB
HHHHHHVGTVGTGGGSGGASTALGLNDIFEAQKIEWHEVGTVGTGGGSGGASTALMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 98 - protein, 1316 aa, synthetic construct
  REGION 1..1316: note = His Tag joined via a linker to a dimer of truncated SSB joined via a linker to the C-terminus of wild type R2 enzyme
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTARRRGGGVGTGGGSGGGTGGGSMEEKVGNLKPNMESVNVTVRVLEASEARQIQTKNGVRTISEAIVGDETGRVK

SEQ ID NO: 99 - protein, 1381 aa, synthetic construct
  REGION 1..1381: note = His Tag joined via a linker to a dimer of truncated SSB joined via a linker to the N-terminus of wild type R2 enzyme
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTARRRGGGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTARRRVGTGGGSGGGTGGGSMMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 100 - protein, 1096 aa, synthetic construct
  REGION 1..1096: note = His Tag joined via a linker to a dimer of truncated SSB joined via a linker to the N-terminus of deltaR2 enzyme
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTARRRGGGVGTGGGSGGGTGGGSMEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTARRRVGTGGGSGGGTGGGSKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 101 - protein, 17 aa, synthetic construct
  REGION 1..17: note = Synthetic
VGTVGTGGGSGGASTAL

SEQ ID NO: 102 - protein, 22 aa, synthetic construct
  REGION 1..22: note = Synthetic
VGTVGTGGGSEAAAKGGASTAL

SEQ ID NO: 103 - protein, 19 aa, synthetic construct
  REGION 1..19: note = Synthetic
VGTGGGSEAAAKGGASTAL

SEQ ID NO: 104 - protein, 25 aa, synthetic construct
  REGION 1..25: note = Synthetic
VGTGGGSGGGEAAAKEAAAKSGGGS

SEQ ID NO: 105 - protein, 26 aa, synthetic construct
  REGION 1..26: note = Synthetic
VGTGGGSGGGEAAAKEAAAKSGGGSA

SEQ ID NO: 106 - protein, 15 aa, synthetic construct
  REGION 1..15: note = Synthetic
VGTGGGSGGGTGGGS

SEQ ID NO: 107 - protein, 16 aa, synthetic construct
  REGION 1..16: note = Synthetic
VGTGGGSGGGTGGGSA

SEQ ID NO: 108 - protein, 4 aa, synthetic construct
  REGION 1..4: note = Synthetic
GGGS

SEQ ID NO: 109 - protein, 5 aa, synthetic construct
  REGION 1..5: note = Synthetic
GGGGS

SEQ ID NO: 110 - protein, 5 aa, synthetic construct
  REGION 1..5: note = Synthetic
EAAAK

SEQ ID NO: 111 - DNA, 522 bp, synthetic construct
  misc_feature 1..522: note = Synthetic
  source 1..522: mol_type = other DNA
tggacattaattagggctgaaagccctaacttaatggacgggaggtatcccaataggaggtttcctcctatggttttcaaaacaatcaccatcatgctattaatgatattaaaatcccaactataccaaagaatatcccaattatccataaaactgtaactaagtgaggctctctcattggtttatacttcaatataagccttggtagggatagatagccacctatatagtatagcttcccatcttctttgagagttgttggtttatgctcatccctactcataaccccagcacttagatattttaaagaggcatctatcacataaggcatcattataactaaaaatgggatatattccttataaactactgctaagacagctaagaaagctccaattggtagagttccaacatctcctggaaaaacctttgctggatatttgttaaatatcaatagccctaaataggatgcagagaatatcaaagcggaaaaaatccaaaaaaaaaaaaaaaaaaaaaaaa

SEQ ID NO: 112 - DNA, 1061 bp, synthetic construct
  misc_feature 1..1061: note = Synthetic
  source 1..1061: mol_type = other DNA
tccagattacttccatttccgcccaagctgctcacagtatacgggcgtcggcatccagaccgtcggctgatcgtggttttactaggctagactagcgtacgagcactatggtcagtaattcctggaggaataggtaccaagaaaaaaacgaacctttgggttccagagctgtacggtcgcactgaactcggataggtctcagaaaaacgaaatataggcttacggtaggtccgaatggcacaaagcttgttccgttagctggcataagattccatgcctagatgtgatacacgtttctggaaactgcctcgtcatgcgactgttccccggggtcagggccgctggtatttgctgtaaagaggggcgttgagtccgtccgacttcactgccccctttcagccttttgggtcctgtatcccaattctcagaggtcccgccgtacgctgaggaccacctgaaacgggcatcgtcgctcttcgttgttcgtcgacttctagtgtggagacgaattgccagaattattaactgcgcagttagggcagcgtctgaggaagtttgctgcggtttcgccttgaccgcgggaaggagacataacgatagcgactctgtctcaggggatctgcatatgtttgcagcatactttaggtgggccttggcttccttccgcagtcaaaaccgcgcaattatccccgtcctgatttactggactcgcaacgtgggtccatcagttgtccgtataccaagacgtctaagggcggtgtacacccttttgagcaatgattgcacaacctgcgatcaccttatacagaattatcaatcaagctccccgaggagcggacttgtaaggaccgccgctttcgctcgggtctgcgggttatagcttttcagtctcgacgggctagcacacatctggttgactaggcgcatagtcgccattcacagatttgctcggcaatcagtactggtaggcgttagaccccgtgactcgtggctgaacggccgtacaactcgacagccggtgcttgcgttttacccttaaaaaaaaaaaaaaaaaaaaaaaa

SEQ ID NO: 113 - DNA, 33 bp, synthetic construct
  misc_feature 1..33: note = Synthetic
  source 1..33: mol_type = other DNA
acactctttccctacacgacgctcttccgatct

SEQ ID NO: 114 - DNA, 34 bp, synthetic construct
  misc_feature 1..34: note = Synthetic
  source 1..34: mol_type = other DNA
gtgactggagttcagacgtgtgctcttccgatct

SEQ ID NO: 115 - DNA, 23 bp, synthetic construct
  misc_feature 1..23: note = Synthetic
  source 1..23: mol_type = other DNA
acactctttccctacacgacgct

SEQ ID NO: 116 - DNA, 24 bp, synthetic construct
  misc_feature 1..24: note = Synthetic
  source 1..24: mol_type = other DNA
ctgagacctatccgagttcagtgc

SEQ ID NO: 117 - DNA, 32 bp, synthetic construct
  misc_feature 1..32: note = Synthetic
  source 1..32: mol_type = other DNA
gtggctatctatccctaccaaggcttatattg

SEQ ID NO: 118 - protein, 4 aa, synthetic construct
  REGION 1..4: note = Synthetic
GSGS

SEQ ID NO: 119 - protein, 19 aa, synthetic construct
  REGION 1..19: note = Synthetic
GGGGSGGGGSGAGSGGGGS

SEQ ID NO: 120 - protein, 11 aa, synthetic construct
  REGION 1..11: note = Synthetic
GGGGGSGGGGS

SEQ ID NO: 121 - protein, 6 aa, synthetic construct
  REGION 1..6: note = Synthetic
GGGGSG

SEQ ID NO: 122 - protein, 4 aa, synthetic construct
  REGION 1..4: note = Synthetic
GSGG

SEQ ID NO: 123 - protein, 5 aa, synthetic construct
  REGION 1..5: note = Synthetic
GSGGG

SEQ ID NO: 124 - protein, 8 aa, synthetic construct
  REGION 1..8: note = Synthetic
DYKDDDDK